(19) United States
(12) Patent Application Publication
(10) Pub. No.: US 2026/0083525 A1
(43) Pub. Date: Mar. 26, 2026

(54) HAND CONTROLLERS, SYSTEMS, AND CONTROL METHODS FOR SURGICAL ROBOTIC SYSTEMS

(71) Applicant: VICARIOUS SURGICAL INC., Waltham, MA (US)

(72) Inventors: Theodore M. Aronson, Someville, MA (US); Tabitha A. Solomon, Arlington, MA (US); Michael Cattafe, Concord, MA (US); Katherine Lopez Hayna, Belmont, MA (US); Sophia Atik, Medford, MA (US); William Vespa, Quincy, MA (US); Emma Morgan, Boston, MA (US)

(21) Appl. No.: 19/111,232

(22) PCT Filed: Sep. 29, 2023
(86) PCT No.: PCT/US2023/034203

§ 371 Date: Mar. 12, 2025

Related U.S. Application Data
(60) Provisional application No. 63/412,372, filed on Sep. 30, 2022.
(60) Provisional application No. 63/412,377, filed on Oct. 1, 2022.

(51) Int. Cl. A61B 34/00; A61B 34/37

(52) U.S. Cl. CPC A61B 34/74; A61B 34/37

(57) ABSTRACT

Systems and methods employing at least one hand controller for controlling a robotic assembly of a surgical robotic system are provided. The hand controller may be used in a surgeon console of a surgical robotic system. The hand controller may include a contoured housing having an upper surface, an inside side surface adjacent the upper surface, an outside side surface facing away from the first side surface, and a lower surface facing away from the upper surface. The hand controller may include a plurality of buttons including a first button positioned on the upper surface and a second button positioned on one of the upper surface, the inside side surface, or the outside side surface; a first touch input device positioned on the upper surface; and a first paddle mounted to either of the inside side surface or the outside side surface.

### RELATED APPLICATIONS

This application claims the benefit of the filing date, under 35 U.S.C. § 119(e), of U.S. Provisional Application No. 63/412,372, filed on Sep. 30, 2022, and U.S. Provisional Application No. 63/412,377, filed on Oct. 1, 2022, the entire contents of which are incorporated herein by reference.

### BACKGROUND

Surgical robotic systems permit a surgeon (also described herein as an “operator” or a “user”) to perform an operation using robotically-controlled instruments to perform tasks and functions during a procedure. The surgeon may use a visualization system to view or watch the operation, including to view images from cameras showing the patient and/or mounted to the robotically-controlled instruments.

Controls are provided to permit the user to control features of the surgical robotic system, such as surgical tools, arms of the surgical robotic system and camera position, as well as to navigate through features and settings available within the surgical robotic systems. Controls to be used for a surgical robotic system may include one or more hand controllers to be operated by a hand or hands of the surgeon.

Certain controls may suffer from a number of deficiencies. For example, certain control systems may fail to provide sufficient inputs to control aspects to the surgical robotic systems or may fail to provide user-friendly operation or “natural” feel for the user. These deficiencies may result in a user having difficulty operating the surgical robotic system, reduced accuracy and ease of operation, or decreased user satisfaction.

### SUMMARY

The present disclosure is directed to one or more hand controllers for controlling a surgical robotic system including a robotic assembly configured to be disposed with an internal body cavity of a subject. Each of the one or more hand controllers may include a contoured housing having an upper surface, an inside side surface adjacent the upper surface, an outside side surface facing away from the inside side surface, and a lower surface facing away from the upper surface; a plurality of buttons including a first button positioned on the upper surface and a second button positioned on one of the upper surface, the inside side surface, or the outside side surface; a first touch input device positioned on the upper surface; and a first paddle disposed at either of the inside side surface or the outside side surface. The contoured housing is configured to be grasped with a user's thumb on the inside side surface and at least some of the user's fingers, the user's palm, or both over the upper surface, and the first button is configured to be manipulated by the user's pointer finger or index finger.

In some embodiments, for each of the one or more hand controllers, a contact surface of the first paddle is disposed at the outside side surface of the housing. In some embodiments, each of the one or more hand controllers also includes a second paddle. A contact surface of the first paddle may be disposed at the outside side surface of the housing and a contact surface of the second paddle may be disposed at the inside side surface of the housing, or the contact surface of the first paddle may be disposed at the inside side surface of the housing and the contact surface of the second paddle is disposed at the outside side surface of the housing. In some embodiments, the first paddle is engaged with the second paddle via one or more gears. A movement of the first paddle may cause a reciprocal movement of the second paddle, and a movement of the second paddle may cause a reciprocal movement of the first paddle. In some embodiments, for each of the hand controllers, the first touch input device is a scroll wheel, a rocker button, a joy stick, a pointing stick, a touch pad, or a trackball. In some embodiments, at least one of the one or more hand controllers also includes a second touch input device. In some embodiments, for each of the hand controllers, deflection of the first paddle is configured to produce a signal that the surgical robotic system uses as an input to control the robotic subassembly.

For some embodiments, for at least one of the hand controllers, at least one of the plurality of buttons maps to a function selected from a plurality of functions. The plurality of functions may include triggering activation of a clutch with respect to a hand controller where the activation of the clutch associated with a hand controller permits or enables movement of the respective hand controller without causing movement of the robotic assembly. The plurality of functions may also include resetting a pose of the robotic assembly with respect to robotic arms and a virtual chest of the robotic assembly to a default pose while maintaining a position and an orientation of an instrument tip for each of the robotic arms. The plurality of functions may also include activating or exiting an instrument control mode of the surgical robotic system, where a movement of one of the hand controllers when in the instrument mode causing a corresponding movement in a corresponding robotic arm of the robotic assembly. The plurality of functions may also include activating or exiting a scan mode of the surgical robotic system, where a movement of one of the hand controllers when in the scan mode causes a corresponding change in an orientation of a camera assembly of the robotic assembly while the robotic arm of the robotic assembly remains stationary where the robotic assembly includes one robotic arm, or while all robotic arms of the robotic assembly remain stationary where the robotic assembly includes more than one robotic arm. The plurality of functions may also include activating or exiting a view mode of the surgical robotic system, where a movement of one of the hand controllers when in the view mode causes a corresponding change in the orientation of and in a position of the camera assembly while maintaining positions of instrument tips of all robotic arms of the robotic assembly. The plurality of functions may also include activating or exiting a pivot mode of the surgical robotic system, where activating the pivot mode causes a change in an orientation of the camera assembly or a change in the orientation of the camera assembly and a change in an orientation of the virtual chest to center a view of the camera assembly on a position of a midpoint between instrument tips of the robotic arms, which is an average instrument tip position, and a movement of one of the hand controllers when the pivot mode is activated causing a corresponding movement of an instrument tip of a corresponding robotic arm relative to the view of the camera assembly and causes a change in the orientation of the camera assembly or the change in the orientation of the camera assembly and the change in the orientation of the virtual chest to maintain the view of the camera assembly centered on the average instrument tip position. The plurality of functions may also include activating or exiting a menu feature of the surgical robotic system, where the menu feature causes a menu to be displayed in a graphical user interface of the surgical robotic system and input from a touch input device or a button of the one or more hand controllers being used for selection of an item of the menu. The plurality of functions may also include navigating between and/or selecting options from the menu displayed in the graphical user interface of the surgical robotic system. The plurality of functions may also include activating or exiting an elbow bias feature, where the elbow bias feature enables adjustment of a magnitude and a direction of an elevation of an elbow of a robotic arm while maintaining a position of an instrument tip of the robotic arm for each robotic arm of the robotic assembly. The plurality of functions may also include controlling an adjustment of an elevation of an elbow of a robotic arm of the robotic assembly for one robotic arm of the robotic assembly or for each robotic arm of the robotic assembly and changing or selecting a zoom of a view from a camera assembly of the robotic assembly. In some embodiments, the plurality of functions can be grouped in to one or more groups. In some embodiments, the surgical robotic system does not need to include all of the plurality of functions. For example, a pivot mode can been removed from the surgical robotic system and/or functionalities of a pivot mode can be consolidated into a camera mode.

In some embodiments, for at least one of the hand controllers, at least two of the plurality of buttons map to functions selected from the plurality of functions. In some embodiments, for at least one of the hand controllers, at least three of the plurality of buttons maps to functions selected from the plurality of functions. In some embodiments, for at least one of the one or more hand controllers, the first touch input device has one or more functions selected from the plurality of functions. In some embodiments, for each of the one or more hand controllers, the first touch input device has one or more functions selected from the plurality of functions. In some embodiments, at least one of the one or more hand controllers also include a second touch input device; and for at least one of the one or more hand controllers, the first touch input device or the second touch input device has one or more functions selected from the plurality of functions. In some embodiments, at least one of the one or more hand controllers also includes a second touch input device; and for at least one of the one or more hand controllers, the first touch input device and the second touch input device each have one or more functions selected from the plurality of functions. In some embodiments, for at least one of the one or more hand controllers, an engagement of the first button produces a signal triggering activation of a clutch feature of the surgical robotic system with respect to a hand controller, and activation of the clutch feature permits or enables movement of the respective hand controller without causing movement of the robotic assembly. In some embodiments, for each hand controller, the outside side surface of the housing is substantially concave. In some embodiments, each of the one or more hand controllers also includes a thumb pad disposed on either of the inside side surface or the outside side surface. In some embodiments, at least one of the one or more hand controllers includes a sensor configured to sense contact of a hand of the operator with the hand controller or to sense proximity of a hand of the operator to the hand controller. In some embodiments, an input from the sensor is used for activating or exiting an instrument control mode of the surgical robotic system and a movement of one of the hand controllers when in the instrument mode causes a corresponding movement in a corresponding robotic arm of the robotic assembly.

In some embodiments, the present disclosure is directed to one or more hand controllers for controlling a surgical robotic system including a robotic assembly configured to be disposed with an internal body cavity of a subject. Each of the one or more hand controllers may include a contoured housing having an upper surface, an inside side surface adjacent the upper surface, an outside side surface facing away from the inside side surface, and a lower surface facing away from the upper surface. Each of the one or more hand controllers may also include a plurality of buttons including a first button positioned on the upper surface and a second button positioned on one of the upper surface, the inside side surface, or the outside side surface. Each of the one or more hand controllers may also include a first touch input device positioned on the upper surface; a first paddle disposed at the outside side surface. Each of the one or more hand controllers may also include a second paddle disposed at the inside side surface. The contoured housing may be configured to be grasped with a user's thumb on the inside side surface and at least some of the user's fingers, the user's palm, or both over the upper surface, and the first button is configured to be manipulated by the user's pointer finger or index finger.

In another aspect, the present disclosure is directed to a hand controller system for controlling a surgical robotic system including a robotic assembly configured to be disposed within an internal body cavity of a subject. The hand controller system may include a first hand controller as described above removably coupled to or configured to be connected to an operator console via a first hand controller support. The hand controller system may also include a second hand controller as described above removably coupled to or configured to be connected to the operator console via a second hand controller support. An inside surface of the first hand controller may face an inside surface of the second hand controller when the first and second hand controllers are in a neutral position in use.

In another aspect, the present disclosure is directed to a hand controller system for controlling a surgical robotic system including a robotic assembly configured to be disposed with an internal body cavity of a subject. The hand controller system may include a first hand controller removably coupled to or configured to be connected to an operator console via a first hand controller support. The first hand controller may include: (i) a contoured housing having an upper surface, a first side surface adjacent the upper surface, a second side surface facing away from the first side surface, and a lower surface facing away from the upper surface; (ii) a first button and a first scroll wheel disposed at the upper surface; (iii) a first paddle having a contact surface disposed at the first side surface or at the second side surface; and (iv) a second button disposed at the first side surface or at the second side surface. The hand controller system may also include a second hand controller removably coupled to or configured to be connected to the operator console via a second hand controller support. The second hand controller may including: (i) a contoured housing having an upper surface, a first side surface adjacent the upper surface, a second side surface facing away from the first side surface, and a lower surface facing away from the upper surface; (ii) a first button and a first scroll wheel disposed at the upper surface; (iii) a first paddle having a contact surface disposed at the first side surface or at the second side surface; and (iv) a second button disposed at the first side surface or at the second side surface. An inside surface of the first hand controller may face an inside surface of the second hand controller when the first and second hand controllers are in a neutral position in use.

In some embodiments, the present disclosure is directed to a surgeon console including: the hand controller system described above; and a plurality of foot pedals. The plurality of foot pedals may include one or more of: a view mode foot pedal, a pivot mode foot pedal, a travel mode foot pedal, and a translate mode foot pedal. The view mode foot pedal may be configured to activate a view mode of the surgical robotic system upon engagement in which a movement of one of the hand controllers when the view mode is activated causes a corresponding change in an orientation of and a position of the camera assembly and a rotation of the virtual chest of the robotic assembly to maintain an angular deviation between a line from the center of the virtual chest to a position of a midpoint between instrument tips of robotic arms of the robotic assembly, which is an average instrument tip position, and the normal to the virtual chest within an acceptable angular deviation range while maintaining positions and orientations of instrument tips of all robotic arms of the robotic assembly. The pivot mode foot pedal may be configured to activate a pivot mode of the surgical robotic system upon engagement in which activating the pivot mode causes a change in an orientation of the camera assembly or a change in the orientation of the camera assembly and a change in an orientation of a virtual chest of the robotic assembly to center a view of the camera assembly on a position of a midpoint between instrument tips of robotic arms of the robotic assembly, which is an average instrument tip position, and a movement of one of the hand controllers when the pivot mode is activated causing a corresponding movement of an instrument tip of a corresponding robotic arm relative to the view of the camera assembly and causing a change in the orientation of the camera assembly or a change in the orientation of the camera assembly and a change in the orientation of the virtual chest to maintain the view of the camera assembly centered on the average instrument tip position and to maintain an angular deviation between a line from the center of the virtual chest to the average instrument tip position and a normal to the virtual chest within an acceptable angular deviation range. The travel mode foot pedal may be configured to activate a travel mode of the surgical robotic system upon engagement in which a movement of one of the hand controllers when the travel mode is activated may cause a corresponding movement of an instrument tip of a corresponding robotic arm by a corresponding first movement including a corresponding first translation and a corresponding first rotation relative to the view of the camera assembly, causing the camera assembly to rotate to center the view of the camera assembly on the average tip position, and causing a virtual chest of the robotic assembly to be translated, to be rotated, or both to maintain a distance between a center of the virtual chest of the robotic assembly and the average instrument tip position within an acceptable distance range and to maintain an angular deviation between a line from the center of the virtual chest to the average instrument tip position and a normal to the virtual chest within an acceptable angular deviation range. The translate mode foot pedal may be configured to activate a translate mode of the surgical robotic system upon engagement in which a movement of one of the hand controllers when the translate mode is activated causes a corresponding movement of an instrument tip of a corresponding robotic arm relative to the view of the camera assembly, causing rotation of the camera assembly to center the view of the camera assembly on the average tip position, and causing translation of the virtual chest to maintain a distance between the center of the virtual chest and the average instrument tip position within in an acceptable distance range.

In some embodiments, the present disclosure is directed to a method for controlling a robotic assembly of a surgical robotic system that includes an image display and a right hand controller and a left hand controller configured to sense a movement of an operator's hands. The robotic assembly may include a camera assembly and a robotic arm assembly including a first robotic arm and a second robotic arm each of the first robotic arm and the second robotic arm having an associated instrument tip, positions and orientations of the robotic arms and camera assembly defining a virtual chest of the robotic assembly. The method may including, while the robotic assembly is disposed in an interior cavity of a subject, receiving a first control mode selection input and changing a current control mode of the surgical robotic system to a first control mode in response to the first control mode selection input, the surgical robotic system having a plurality of control modes including one or more of: a scan mode, a view mode, a travel mode, a pivot mode, and a translation mode, and the first control mode being the scan mode, the view mode, the travel mode, the pivot mode, or the translation mode. The method may also include certain actions response to a first movement including a first translation and a first rotation of the right hand controller or the left hand controller while the current control mode is the first control mode. Where the first control mode is the scan mode, the method may also include holding the robotic arm assembly stationary while rotating the camera assembly in a corresponding first rotation relative to a view of the camera assembly displayed on the image display of the surgical robotic system, which is a displayed camera view. Where the first control mode is the travel mode, the method may also include moving the corresponding instrument tip by a corresponding first movement including a corresponding first translation and a corresponding first rotation relative to the displayed camera view while rotating the camera assembly to center the view of the camera assembly on a position of a midpoint between instrument tips of the robotic arms of the robotic assembly, which is an average tip position, while translating the robotic assembly to translate a chest point of the virtual chest, rotating the robotic assembly to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range; where the first control mode is the view mode, holding a position and an orientation of each instrument while moving the camera assembly by a corresponding first movement including a corresponding first translation and a corresponding first rotation relative to the displayed camera view and rotating a virtual chest of the robotic assembly to maintain an angular deviation between the line from the center of the virtual chest to the average instrument tip position and the normal to the virtual chest within the acceptable angular deviation range. Where the first control mode is the pivot mode, the method may also include moving a corresponding instrument tip by a corresponding first movement including a corresponding first rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range. Where the first control mode is the translate mode, the method may also include moving a corresponding instrument tip by a corresponding first movement including a corresponding first translation and a corresponding first rotation relative to the displayed camera view while rotating the camera assembly or rotating the camera assembly and translating the virtual chest to center the view of the camera assembly on the average tip position and to maintain a distance between the center of the virtual chest and the average instrument tip position within the acceptable distance range.

In some embodiments, the current control mode may be an instrument control mode in which a movement of the right hand controller or the left hand controller causes a corresponding movement of an instrument tip at a distal end of a corresponding robotic arm relative to the displayed camera view without moving or changing an orientation of the virtual chest of the robotic assembly. In some embodiments, the first control mode selection input may be received via a foot pedal of the surgical robotic system. In some embodiments, the first control mode selection input may be received via the first hand controller or the second hand controller. In some embodiments, the first control mode selection input may be received via a button or touch input device of the right hand controller or the left hand controller. In some embodiments, the first control mode may be the view mode. In some embodiments, the first control mode may be the scan mode. In some embodiments, the first control mode may be the travel mode. In some embodiments, the first control mode may be the translate mode. In some embodiments, the first control mode may be the pivot mode.

In some embodiments, the method may further include: receiving a second control mode selection input; changing a current control mode of the surgical robotic system to a second control mode different than the first control mode in response to the second control mode selection input, wherein the plurality of control modes of the surgical robotic system includes two or more of the scanning mode, the view mode, and the travel mode, the translate mode, pivot mode; and in response to a second movement including a second translation and a second rotation of the right hand controller or the left hand controller while the current control mode is the second control mode. Where the second control mode is the scan mode, the method includes holding the robotic arm assembly stationary while rotating the camera assembly in a corresponding second rotation relative to the displayed camera view. Where the second control mode is the travel mode, the method includes moving the corresponding instrument tip by a corresponding second movement including a corresponding second translation and a corresponding second rotation relative to the displayed camera view while rotating the camera assembly to center the view of the camera assembly on the an average tip position, while translating the robotic assembly to translate the chest point of the virtual chest, rotating the robotic assembly to rotate the virtual chest, or both, to maintain the distance between the center of the virtual chest and the average tip position in the acceptable distance range, and to maintain an angular deviation between the line from the chest point to the average tip position and the normal to the virtual chest within the acceptable angular deviation range. Where the second control mode is the view mode, the method includes holding a position and an orientation of each instrument while moving the camera assembly by a corresponding second movement including a corresponding second translation and a corresponding second rotation relative to the displayed camera view and rotating the virtual chest of the robotic assembly to maintain the angular deviation between the line from the center of the virtual chest to the average instrument tip position and the normal to the virtual chest within the acceptable angular deviation range. Where the second control mode is the pivot mode, the method includes moving a corresponding instrument tip by a corresponding second movement including a corresponding second rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range. Where the second control mode is the translate mode, the method includes moving a corresponding instrument tip by a corresponding second movement including a corresponding second translation and a corresponding second rotation relative to the displayed camera view while rotating the camera assembly or rotating the camera assembly and translating the virtual chest to center the view of the camera assembly on the average tip position and to maintain the distance between the center of the virtual chest and the average instrument tip position within the acceptable distance range.

In some embodiments, the virtual chest is defined by a chest plane extending between a first pivot point of a most proximal joint of the first robotic arm, a second pivot point of a most proximal joint of the second robotic arm, and a camera imaging center point of the camera assembly. In some embodiments, the method may further include: receiving a first function selection input corresponding to an input function located on the left hand controller, the right hand controller, or both; and in response to receipt of the first function selection input, performing a first function. In some embodiments, performing the first function includes: displaying a menu on a portion of the image display while holding positions and orientations of the camera assembly and the robotic arm assembly constant; resetting a camera assembly orientation and position to align a camera view to an average instrument tip position and to the virtual chest; or activating a clutch for the first hand controller or the second hand controller to enable movement of the respective hand controller without causing movement of the robotic assembly.

In some embodiments, the first function includes displaying the menu on the portion of the image display while holding positions and orientation of the camera assembly and the robotic arm assembly constant; and the method further includes: receiving one or more second function selection inputs corresponding to one or more input functions located on the left hand controller, the right hand controller, or both; and in response receiving the one or more second function selection inputs, graphically indicating options on a portion of the image display for one or more menu item to be selected one a based on the one or more second function inputs, displaying one or more submenus on the portion of the image display corresponding to the one or more second function inputs.

In some embodiments, the method also includes receiving at least one third feature selection input corresponding to an input function located on the left hand controller, the right hand controller, or both, the at least one third feature selection input selecting an option on the image display. Where the at least one third feature selection input selects a zoom level, in response receipt of the at least one third feature selection input, the method may include changing a zoom level of the displayed camera view and storing information regarding the new zoom level for modification of control modes that depend on zoom level. Where the at least one third feature selection input selects an elbow bias level, or selects an elbow bias menu and includes a selection of an elbow bias level from the elbow bias menu, in response receipt of the third feature selection input, the method may include changing an elbow bias of a first robotic arm, of a second robotic arm, or of both.

In some embodiments, the present disclosure is directed to a non-transitory computer readable medium storing instructions that, when executed by one or more processors of a robotic surgical system perform the one of the methods described above.

In some embodiments, the present disclosure is directed to a surgical robotic system for performing a surgery within an internal cavity of a subject, the surgical robotic system including: a right hand controller and a left hand controller operated to manipulate the surgical robotic system; a camera assembly; a robotic arm assembly configured to be inserted into the internal cavity during use, the robotic arm assembly including: a first robotic arm including or coupled to a first instrument tip disposed at a distal end of the first robotic arm; a second robotic arm including or coupled to a second instrument tip disposed at a distal end of the second robotic arm; an image display for outputting an image from the camera assembly; and at least one computing module or control unit. The control unit may be configured to receive movement data from the right hand controller and the left hand controller and in response generate control signals based on a current control mode of the surgical robotic system. The control unit may additionally be configured to receive a control mode selection input and in response change the current control mode of the surgical robotic system to a selected one of a plurality of control modes of the surgical robotic system. The control unit may additionally be configured to receive a function selection input from the first hand controller and/or the left hand controller and generate control signals to perform a corresponding function of a plurality of functions.

In some embodiments, the plurality of control modes includes one or more of: a scan mode, a view mode, a travel mode, a pivot mode, and a translation mode. While the current control mode is the scan mode, in response to a first movement including a first rotation of the right hand controller or the left hand controller, the robotic arm assembly may be held stationary while the camera assembly is rotated in a corresponding first rotation relative to a view of the camera assembly displayed on the image display, which is a displayed camera view. While the current control mode is the travel mode, in response to a first movement including a first translation and a first rotation of the right hand controller or the left hand controller, a corresponding instrument tip may be moved by a corresponding first movement including a corresponding first translation and a corresponding first rotation relative to the displayed camera view while the camera assembly is rotated to center the view of the camera assembly on a position of a midpoint between instrument tips of the robotic arms of the robotic assembly, which is an average tip position, while robotic assembly is translated to translate a chest point of the virtual chest, robotic assembly is rotated to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range. While the current control mode is view travel mode, in response to a first movement including a first translation and a first rotation of the right hand controller or the left hand controller, a position and an orientation of each instrument may be held while the camera assembly is moved by a corresponding first movement including a corresponding first translation and a corresponding first rotation relative to the displayed camera view and a virtual chest of the robotic assembly is rotated to maintain an angular deviation between the line from the center of the virtual chest to the average instrument tip position and the normal to the virtual chest within the acceptable angular deviation range. While the current control mode is the pivot mode, in response to a first movement including a first rotation of the right hand controller or the left hand controller, a corresponding instrument tip may be moved by a corresponding first movement including a corresponding first translation and a corresponding first rotation relative to the displayed camera view, the camera assembly is rotated to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range. While the current control mode is the translate mode, in response to a first movement including a first translation and a first rotation of the right hand controller or the left hand controller, the corresponding instrument tip may be moved by a corresponding first movement including a corresponding first translation and a corresponding first rotation relative to the displayed camera view while camera assembly is rotated or the camera assembly is rotated and the virtual chest is translated to center the view of the camera assembly on the average tip position and to maintain a distance between the center of the virtual chest and the average instrument tip position within the acceptable distance range.

In some embodiments, the plurality of modes also includes an instrument control mode in which a movement of the right hand controller or the left hand controller causes a corresponding movement of an instrument tip at a distal end of a corresponding robotic arm relative to the displayed camera view without moving or changing an orientation of the virtual chest of the robotic assembly. In some embodiments, the plurality of modes includes two or more of the scan mode, the view mode, the travel mode, the pivot mode, and the translation mode. In some embodiments, the plurality of modes includes three or more of the scan mode, the view mode, the travel mode, the pivot mode, and the translation mode. In some embodiments, the plurality of modes includes the scan mode. In some embodiments, the plurality of modes includes the view mode. In some embodiments, the plurality of modes includes the travel mode. In some embodiments, the plurality of modes includes the pivot mode. In some embodiments, the plurality of modes includes the translation mode. In some embodiments, the surgical robotic system includes at least one pedal and the control mode selection input received via the at least one pedal. In some embodiments, the surgical robotic system includes at least one pedal and the control mode selection input is received via the at least one pedal or via the right hand controller or the left hand controller. In some embodiments, the first control mode selection input is received via a button or a touch input device of the right hand controller or the left hand controller or via the at least one pedal. In some embodiments, the virtual chest is defined by a chest plane extending between a first pivot point of a most proximal joint of the left robotic arm, a second pivot point of a most proximal joint of the right robotic arm, and a camera imaging center point of the camera assembly.

In some embodiments, the plurality of functions includes displaying a menu on a portion of the image display while holding positions and orientations of the camera assembly and the robotic arm assembly constant. In some embodiments, the plurality of functions includes displaying a submenu on a portion of the image display. In some embodiments, the plurality of functions includes selecting an option in the menu or submenu displayed on the portion of the image display. In some embodiments, the plurality of functions includes resetting a camera assembly orientation and position to align a camera view to an average instrument tip position and to the virtual chest. In some embodiments, the plurality of functions includes resetting a pose of the robotic assembly with respect to robotic arms and a virtual chest to a default pose while maintaining a position and an orientation of an instrument tip for each of the robotic arms. In some embodiments, the plurality of functions includes activating a clutch with respect to the first hand controller or the second hand controller to enable movement of the respective first hand controller or second hand controller without causing movement of the robotic assembly. In some embodiments, the plurality of functions includes activating or exiting an instrument control mode of the surgical robotic system, a movement of one of the hand controllers when in the instrument mode causing a corresponding movement in a corresponding robotic arm of the robotic assembly. In some embodiments, the plurality of functions includes activating or exiting an elbow bias feature, the elbow bias feature enabling adjustment of a magnitude and a direction of an elevation of an elbow of a robotic arm while maintaining a position of an instrument tip of the robotic arm for each robotic arm of the robotic assembly; controlling an adjustment of an elevation of an elbow of a robotic arm of the robotic assembly for one robotic arm of the robotic assembly or for each robotic arm of the robotic assembly. In some embodiments, the plurality of functions includes changing or selecting a zoom of a view from a camera assembly of the robotic assembly.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6A is a perspective view of a left hand controller for use in an operator console of a surgical robotic system in accordance with some embodiments.

FIG. 6B is a perspective view of a right hand controller for use in an operator console of a surgical robotic system in accordance with some embodiments.

FIG. 7A is a perspective view of a left hand controller for use in an operator console of a surgical robotic system in accordance with some embodiments.

FIG. 7B is a perspective view of a right hand controller for use in an operator console of a surgical robotic system in accordance with some embodiments.

FIGS. 8A-8G are perspective views of hand controllers for use in an operator console of a surgical robotic system demonstrating modes of operation and functionalities in accordance with some embodiments.

FIG. 9A is a perspective view of a left hand controller for use in an operator console of a surgical robotic system in accordance with some embodiments.

FIG. 9B is a perspective view of a right hand controller for use in an operator console of a surgical robotic system in accordance with some embodiments.

FIG. 10 is a perspective view of a hand controller for use in an operator console of a surgical robotic system in accordance with some embodiments.

FIG. 11A schematically depicts a top view of a cross-section of a hand controller with paddles in a first configuration and employing a linear sensor to determine a configuration of the paddles in accordance with some embodiments.

FIG. 11B schematically depicts a top view of a cross-section of the hand controller of FIG. 11A with paddles in a second more closed configuration in accordance with some embodiments.

FIG. 12A schematically depicts a top view of a cross-section of a hand controller with paddles in a first configuration employing a linear sensor disposed more distal to an end of the hand controller to determine a configuration of the paddles in accordance with some embodiments.

FIG. 12B schematically depicts a top view of a cross-section of the hand controller of FIG. 12A with paddles in a second more closed configuration in accordance with some embodiments.

FIG. 13A schematically depicts a top view of a cross-section of a hand controller with paddles in a first configuration and employing a rotary sensor to determine a configuration of the first paddle and the second paddle accordance with some embodiments.

FIG. 13B schematically depicts a top view of a cross-section of the hand controller of FIG. 13A with paddles in a second more closed configuration in accordance with some embodiments.

FIG. 14A schematically depicts a top view of a cross-section of a hand controller with paddles in first configuration and extending in a proximal direction and with a rotary sensor disposed at a distal end of the hand controller to determine a configuration of the first paddle and the second paddle accordance with some embodiments.

FIG. 14B schematically depicts a top view of a cross-section of the hand controller of FIG. 14A with paddles in a second more open configuration in accordance with some embodiments.

FIG. 15A is a perspective view of a hand controller in accordance with some embodiments.

FIG. 15B is an inner side view of the hand controller of FIG. 12A.

FIG. 15C is a top view of the hand controller of FIG. 12A.

FIG. 15D is an outer side view of the hand controller of FIG. 12A

FIG. 16A is a perspective view of a hand controller in accordance with some embodiments.

FIG. 16B is a perspective view of a right hand controller and a left hand controller each having a structure like that of FIG. 16A with different functions mapped to buttons and touch input devices in accordance with some embodiments.

FIG. 16C is a perspective view of a right hand controller and a left hand controller each having a structure like that of FIG. 16A with different functions mapped to buttons and touch input devices in accordance with some embodiments.

FIG. 17 schematically depicts a graphical user interface (GUI) displayed to an operator in accordance with some embodiments.

FIG. 18A is a perspective view of a left hand controller for use in connection with an operator console of a surgical robotic system in accordance with some embodiments.

FIG. 18B is a perspective view of a right hand controller for use in connection with an operator console of a surgical robotic system in accordance with some embodiments.

FIG. 19A is a perspective view of a left hand controller for use in connection with an operator console of a surgical robotic system in accordance with some embodiments.

FIG. 19B is a perspective view of a right hand controller for use in connection with an operator console of a surgical robotic system in accordance with some embodiments.

FIG. 20 schematically depicts a graphical user interface including a camera view portion displaying a view from a camera assembly and a menu.

FIG. 21 is a feature map table depicting functions accessed by hand controllers, foot pedals and a menu.

FIG. 22 is a flowchart illustrating steps for controlling a robotic assembly using hand controllers carried out by a surgical robotic system in accordance with some embodiments.

FIG. 23 schematically depicts the trocar plane and various points, and directions associated with a virtual chest and camera assembly of the robotic assembly and with a trocar in accordance with some embodiments.

FIG. 24 schematically depicts positioning and orienting the chest plane to keep the distance from the chest point to the average instrument tip position in acceptable range and to keep an angular deviation of the average instrument tip position from the chest normal/chest direction in an acceptable range in accordance with some embodiments.

FIG. 25 is a perspective view of an operator console of a surgical robotic system featuring a foot pedal array in accordance with some embodiments.

FIG. 26 is a perspective view of the foot pedal array of the operator console depicted in FIG. 25 in accordance with some embodiments.

FIG. 27 is a perspective view of another operator console of a surgical robotic system featuring a foot pedal array including a different arrangement of the fifth foot pedal and sixth foot pedal in accordance with some embodiments.

FIG. 28 is a perspective view of a foot pedal array for an operator console of a surgical robotic system including both column sensors and a row sensor in accordance with some embodiments.

FIG. 29 is a side view of portions of the foot pedal of FIG. 27 illustrating an operator foot interrupting a sensor beam in accordance with some embodiments.

FIG. 30 schematically depicts a graphical user interface (GUI) for an operator including a central area for displaying an image based on image input from the camera assembly.

### DETAILED DESCRIPTION

While various embodiments of the invention have been shown and described herein, it will be clear to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some exemplary embodiments may be described herein or in documents incorporated by reference as employing a plurality of units to perform exemplary processes, it is understood that exemplary processes may also be performed by one or a plurality of modules. Additionally, it is understood that the term controller/control unit may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or control units or multiple different types of controllers or control units may be employed in performing one or more processes. In some embodiments, different controllers or control units may be implemented in different portions of a surgical robotic systems.

Embodiments provide a hand controller for receiving input from an operator to control a robotic assembly of a surgical robotic system, an operator console including such a hand controller, and surgical robotic systems including such hand controllers and operator consoles, methods for controlling a robotic assembly of a surgical robotic system, a surgical robotic system configured to implement control methods, and a computer readable medium including instructions for implementing control methods.

Some advantages of embodiments employing hand controllers as disclosed herein include providing increased input options for the user, increased ease of access to modes and controls of the surgical robotic device, providing improved ergonomics and comfort for the user, and increasing the users' degree of control over the surgical robotic system. Further advantages of some embodiments of the present technology may include an increase in the speed and efficiency in operator control of the system as a result of easier access to modes and controls. This may reduce the overall time of a surgical procedure providing patient health benefits.

Some embodiments disclosed herein are implemented on, employ, or are incorporated into a surgical robotic system that includes a camera assembly having at least three articulating degrees of freedom and two or more robotic arms each having at least six articulating degrees of freedom and an additional degree of freedom corresponding to the movement of an associated end-effector (e.g., grasper, manipulator, and the like). In some embodiments, the camera assembly when mounted within a subject (e.g., a patient) can be moved or rotated in a pitch or yaw direction about 180 degrees such that the camera assembly can view rearwardly back towards the insertion site. As such, the camera assembly and the robotic arms can view and operate dexterously forward (e.g., away from the insertion site), to each side, in an upward or downward direction, as well as in the rearward direction to view backwards towards the insertion site. The robot arms and the camera assembly can also move in the roll, pitch and yaw directions.

Some embodiments described herein provide methods and systems employing multiple different control modes for controlling a surgical robotic system when a robotic assembly of the surgical robotic system is disposed within an internal body cavity of a subject. In some embodiments, the robotic assembly includes at least two robotic arms, which may be described as a “robotic arm assembly” or “arm assembly” herein. In some embodiments, the robotic assembly also includes a camera assembly, which may be also be referred to as a “surgical camera assembly”, or “robotic camera assembly” herein.

Each control mode uses sensed movement of one or more hand controllers, and may also use input from one or more foot pedals, to control the robotic arm assembly and/or the camera assembly. A control mode may be changed from a current control mode to a different selected control mode based on operator input (e.g., provided via one or more hand controllers and/or a pedal of the surgical robotic system). In different control modes, the same movements of the hand controllers may result in different motions of the robotic assembly.

Some embodiments employ a plurality of control modes including an instrument control mode, which may also be referred to as an “instrument mode” herein, as well as one or more of a view control mode, which may also be referred to as a “view mode”, a “camera control mode”, a “camera mode”, a “framing control mode”, a “framing mode,” or a “perspective mode” herein, a scan mode, which may also be referred to herein as a “scanning mode” or a “survey mode”, and a travel control mode, which may also be referred to as a “travel mode” or an “autotrack mode” herein. The pivot mode, the travel mode and the translate mode may all be referred to as “tracking modes” herein.

Some embodiments employ additional features for controlling the robotic assembly. For example, some embodiments enable individual control of an elbow bias or an elbow elevation of a right robotic arm and a left robotic arm. Some embodiments employ a graphical user interface that identifies a current control mode of the surgical robotic system. Some embodiments employ a menu feature in which a menu is displayed on the graphical user interface and one or more of the hand controllers can be used to traverse menu options and select menu options.

Control modes and methods described herein may be particularly advantageous in a surgical robotic system having greater maneuverability than a conventional system. For example, many conventional surgical robotic systems having two robotic arms and fewer degrees of freedom per arm may not be able to change a position or an orientation of a virtual chest of the robotic assembly while keeping instrument tips of end effectors of the robotic arms stationary. As another example, cameras of many conventional surgical robotic systems may have only have degrees of freedom associated with movement of a support for the camera extending through a trocar and may have no independent degrees of freedom for movement relative to the support.

The large number of degrees of freedom in some surgical robotic systems described herein, in comparison to some conventional surgical robotic systems, enables movements of a robotic arm assembly and orientations of a robotic arm assembly not possible with some conventional surgical robotic arms and enables movements of a camera of a robotic camera assembly not possible in cameras for some conventional robotic surgical systems.

Some embodiments described herein provide methods and systems employing multiple different control modes for controlling a surgical robotic system including a robotic assembly configured to be disposed within an internal body cavity of a subject. The robotic assembly may include at least two robotic arms, which may be described as a robotic arm assembly or “arm assembly” herein. The robotic assembly may also include a camera assembly, which may be also be described as a “surgical camera assembly”, or “robotic camera assembly” herein. Each control mode uses sensed movement of one or more hand controllers to control the robotic arm assembly and/or the camera assembly. A control mode may be changed from a current control mode to a different selected control mode based on operator input. In different control modes, the same movements of the hand controllers result in different motions of the robotic assembly.

In some methods and systems, the multiple different control modes, which may be described as a plurality of control modes, include at least one view control mode, also referred to herein as a camera control mode, in which a position and/or an orientation of: at least a portion of the camera assembly, or at least a portion of the robotic arm assembly and at least a portion of the camera assembly are changed in response to movement of at least one of the hand controllers while maintaining stationary positions of instrument tips of end effectors disposed at distal ends of the robotic arms. In some systems and methods, the multiple different control modes include at least one travel control mode. In the travel mode, which is a tracking mode, the chest can be translated, the arms and the chest together can be translated, an orientation of the chest can be changed or any combination of the aforementioned, to center the camera view on the instrument tips. In a camera/view mode, the surgical robotic system can rotate the camera assembly, can translate the chest, and can pivot the chest to change an orientation of a direction of view and a perspective in response to movement of hand controllers while a stationary position of instrument tips of the robotic arms. In the translation mode, which is a tracking mode, the chest or the chest and the arms can be translated together while the camera view is centered on the instrument tips. In the pivot view, which is a tracking mode, the orientation of the can be changed to center the camera view on the instrument tips.

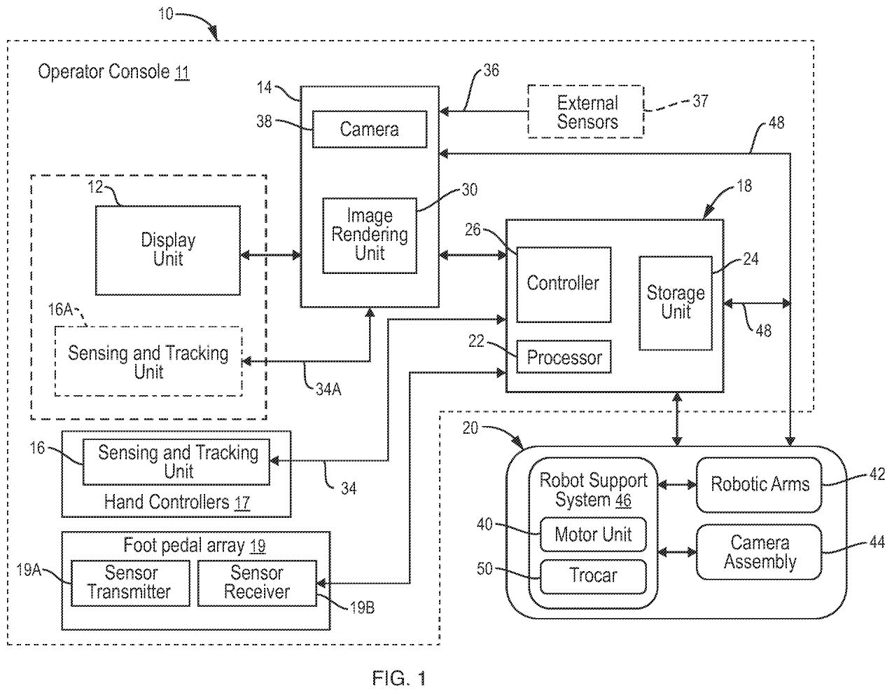

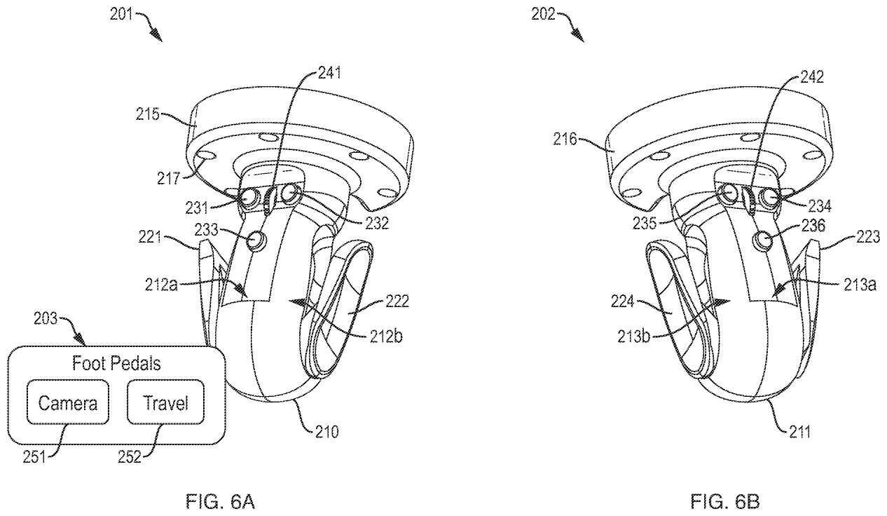

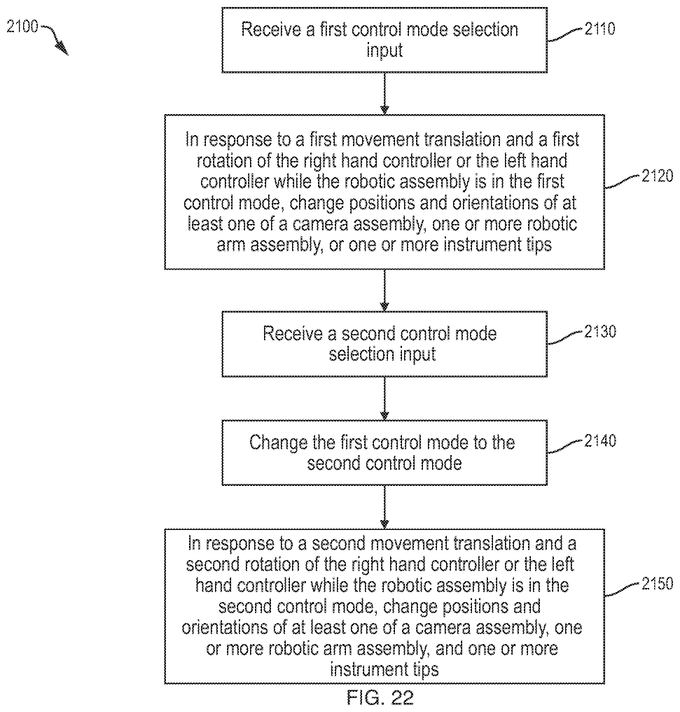

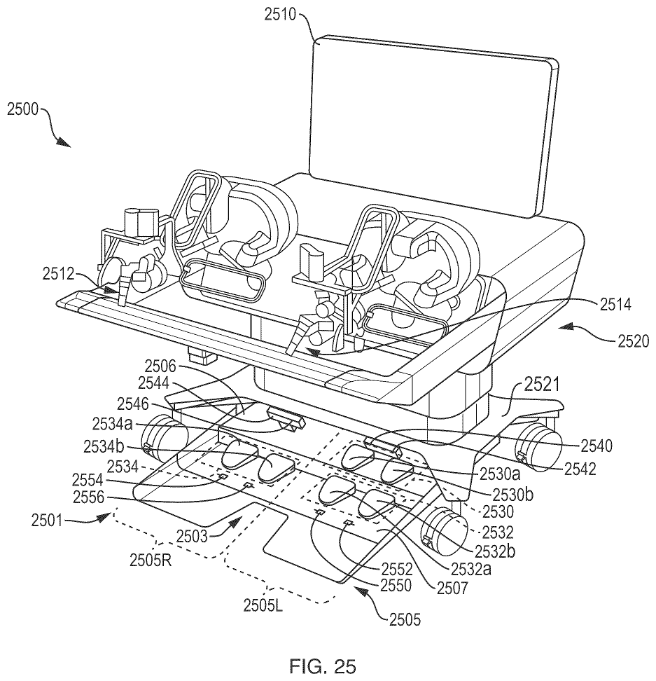

Prior to providing additional specific description of the hand controllers and operator input controls to the operator console with respect to FIGS. 6-21, methods for control of a surgical robotic system with respect to FIGS. 22-24, and foot pedal arrays with respect to FIGS. 25-30, a surgical robotic system in which some embodiments could be employed is described below with respect to FIGS. 1-5.

### Surgical Robotic Systems

Some embodiments may be employed with a surgical robotic system. A system for robotic surgery may include a robotic subsystem. The robotic subsystem includes at least a portion, which may also be referred to herein as a robotic assembly herein, that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic unit, a surgical robotic module or a robotic assembly herein. The surgical robotic unit or surgical robotic module can include multiple different subunits or parts that may be inserted into the trocar separately. The surgical robotic unit, surgical robotic module or robotic assembly can include multiple separate robotic arms that are deployable within the patient along different or separate axes. These multiple separate robotic arms may be collectively referred to as a robotic arm assembly herein. Further, a surgical camera assembly can also be deployed along a separate axis. The surgical robotic unit, surgical robotic module, or robotic assembly may also include the surgical camera assembly. Thus, the surgical robotic unit, surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor unit and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display device or unit 12, an image computing unit 14, which may be a three-dimensional (3D) computing unit, hand controllers 17 having a sensing and tracking unit 16, and a computing unit 18. Additionally, the operator console 11 may include a foot pedal array 19 including a plurality of pedals.

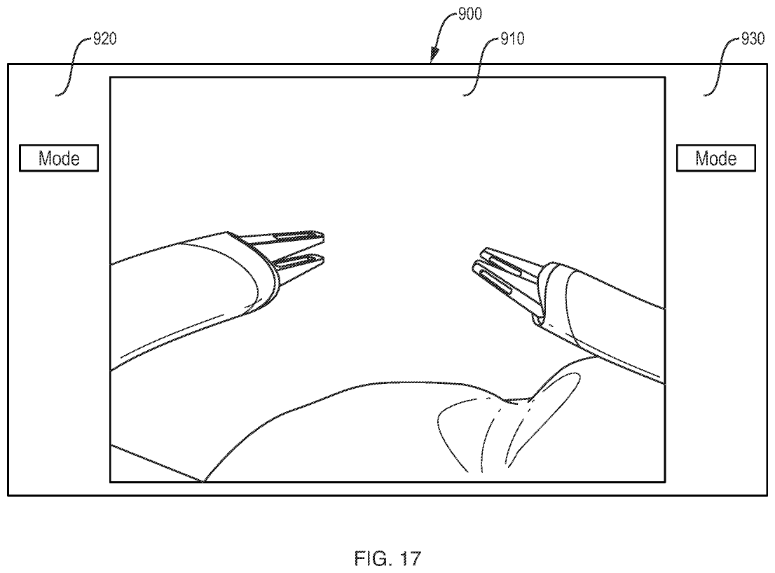

The display unit 12 may be any selected type of display for displaying information, images or video generated by the image computing unit 14, the computing unit 18, and/or the robotic subsystem 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 (see FIG. 17) of the robotic subsystem 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuitry, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware. In some embodiments, the optional sensor and tracking unit 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and robotic arms 42 of the robotic subsystem 20. The tracking and position data 34 generated by the sensing and tracking unit 16 can be conveyed to the computing unit 18 for processing by at least one processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage unit 24. The tracking and position data 34 and 34A can also be used by the control unit 26, which in response can generate control signals for controlling movement of the robotic arms 42 and/or the camera assembly 44. For example, the control unit 26 can change a position and/or an orientation of at least a portion of the camera assembly 44, of at least a portion of the robotic arms 42, or both. In some embodiments, the control unit 26 can also adjust the pan and tilt of the camera assembly 44 to follow the movement of the operator's head.

The robotic subsystem 20 can include a robot support system (RSS) 46 having a motor unit 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50 or a trocar mount. The RSS 46 can further include a support member that supports the motor unit 40 coupled to a distal end thereof. The motor unit 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor unit 40 that is coupled to the robotic subsystem 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robotic arms 42 and the cameras assembly 44 separately or together. The motor unit 40 can also provide mechanical power, electrical power, mechanical communication, and electrical communication to the robotic arms 42, the camera assembly 44, and/or other components of the RSS 46 and robotic subsystem 20. The motor unit 40 can be controlled by the computing unit 18. The motor unit 40 can thus generate signals for controlling one or more motors that in turn can control and drive the robotic arms 42, including for example the position and orientation of each articulating joint of each robotic arm, as well as the camera assembly 44. The motor unit 40 can further provide for a translational or linear degree of freedom that is first utilized to insert and remove each component of the robotic subsystem 20 through a trocar 50. The motor unit 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robotic arms 42, the camera assembly 44, and the like), and for generating control signals in response thereto. The motor unit 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking unit 16A for tracking at least a portion of the operator's head.

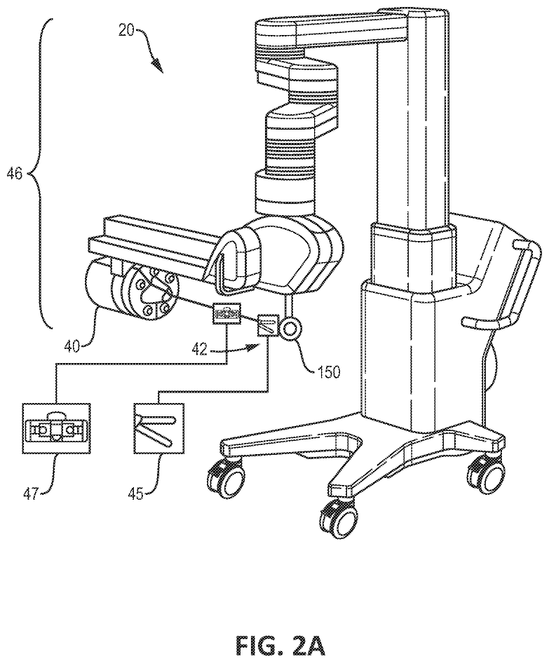

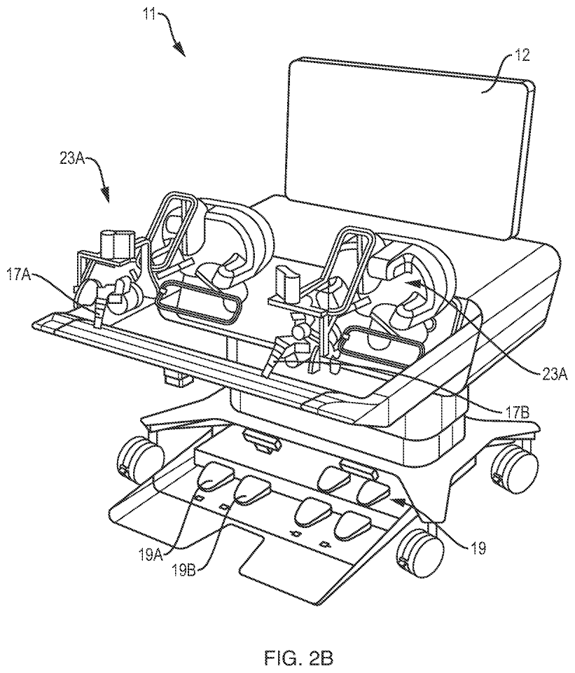

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic assembly 20 includes the RSS 46, which, in turn includes the motor unit 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display unit 12, hand controllers 17, and also includes one or more additional controllers, such as a foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasable connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

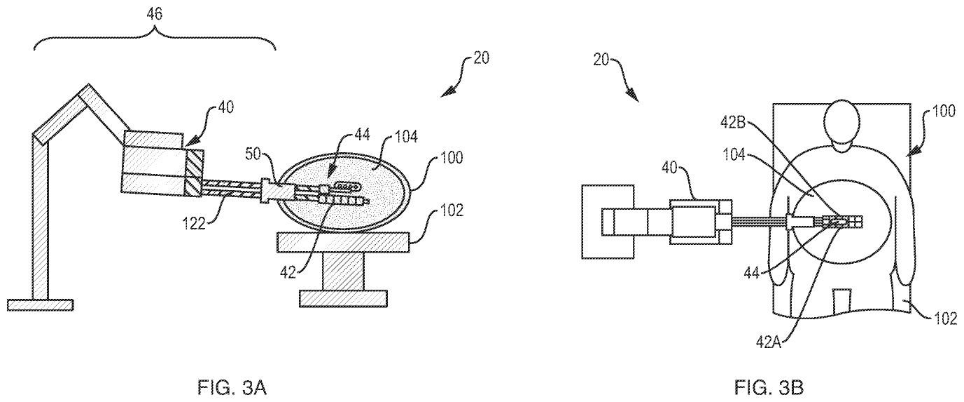

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor unit 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

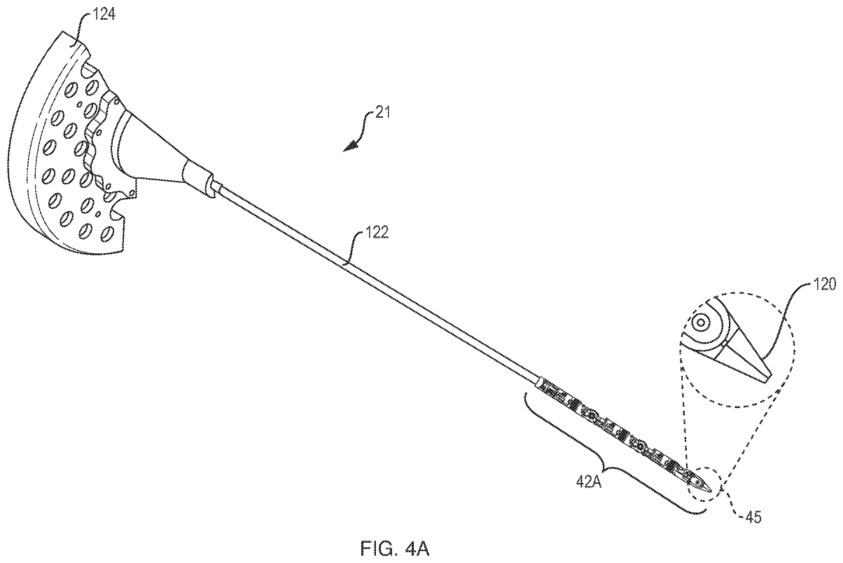

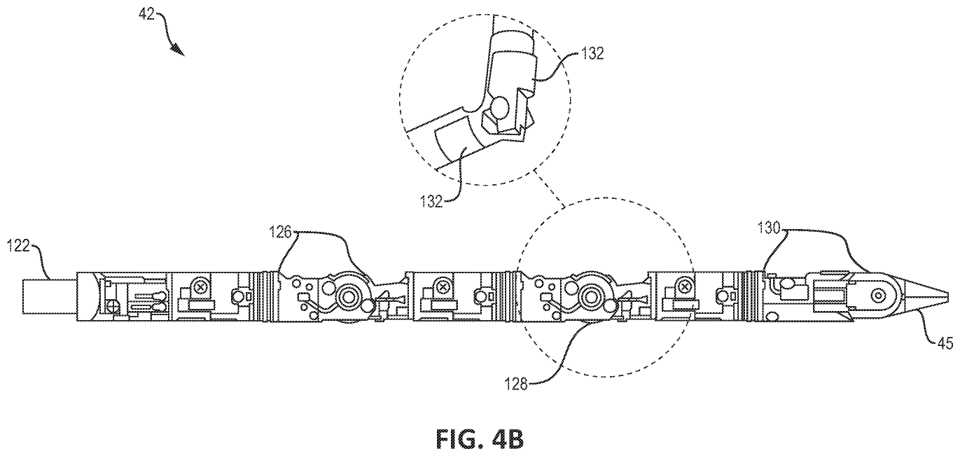

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor unit 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

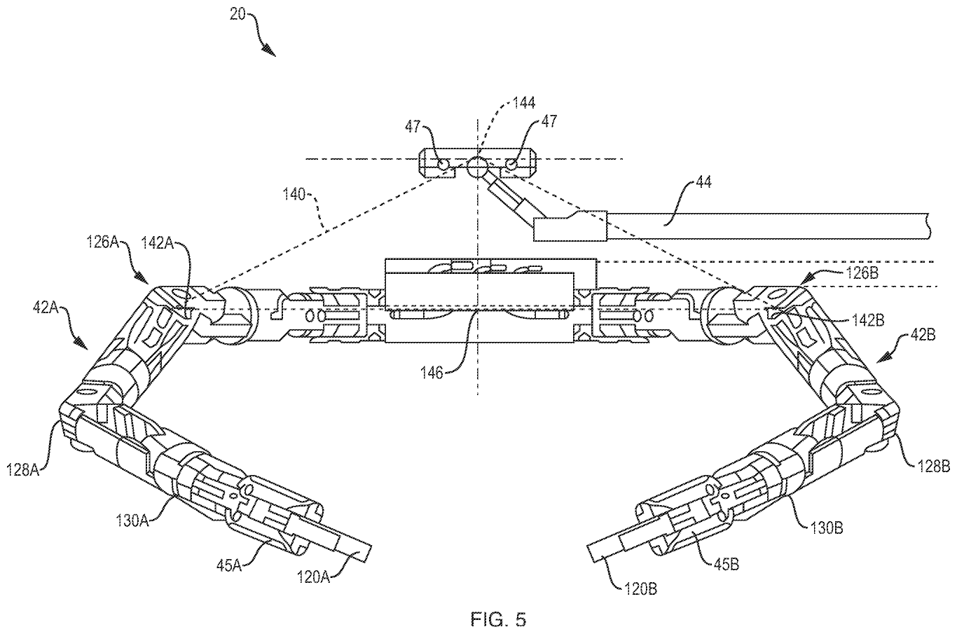

FIG. 5 illustrates a perspective front view of a portion of the robotic assembly 20 configured for insertion into an internal body cavity of a patient. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest.

In some embodiments, sensors in one or both of the first robotic arm 42A and the second robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Hand Controllers for a Surgical Robotic System

Hand controllers for a surgical robotic system as described herein can be employed with any of the surgical robotic systems described above or any other suitable surgical robotic system. Further, some embodiments of hand controllers described herein may be employed with semi-robotic endoscopic surgical systems that are only robotic in part.

As explained above, controllers for a surgical robotic system may desirably feature sufficient inputs to provide control of the system, an ergonomic design and “natural” feel in use.

A hand controller, hand controller system, and a surgeon console or operator console employing a hand controller may be understood with reference to embodiments depicted in FIGS. 6-16, 18, and 19 as described below.

In some embodiments described herein, reference is made to a left hand controller and a corresponding left robotic arm, which may be a first robotic arm, and to a right hand controller and a corresponding right robotic arm, which may be a second robotic arm. In some embodiments, a robotic arm considered a left robotic arm and a robotic arm considered a right robotic arm may change due to a configuration of the robotic arms and the camera assembly being adjusted such that the second robotic arm corresponds to a left robotic arm with respect to a view provided by the camera assembly and the first robotic arm corresponds to a right robotic arm with respect view provided by the camera assembly. In some embodiments, the surgical robotic system changes which robotic arm is identified as corresponding to the left hand controller and which robotic arm is identified as corresponding to the right hand controller during use. In some embodiments, at least one hand controller includes one or more operator input devices to provide one or more inputs for additional control of a robotic assembly. In some embodiments, the one or more operator input devices receive one or more operators inputs for at least one of: engaging a scanning mode, resetting a camera assembly orientation and position to a align a view of the camera assembly to the instrument tips and to the chest; displaying a menu, traversing a menu or highlighting options or items for selection and selecting an item or option, selecting and adjusting an elbow position, and engaging a clutch associated with an individual hand controller. In some embodiments, additional functions may be accessed via the menu, for example, selecting a level of a grasper force (e.g., high/low), selecting an insertion mode, an extraction mode, or an exchange mode, adjusting a focus, lighting, or a gain, camera cleaning, motion scaling, rotation of camera to enable looking down, etc.

FIG. 6A depicts a left hand controller 201 and FIG. 6B depicts a right hand controller 202 in accordance with some embodiments. The left hand controller 201 and the right hand controller 202 each include a contoured housing 210, 211, respectively. Each contoured housing 210, 211, includes an upper surface 212a, 213a, an inside side surface 212b, 213b adjacent the upper surface, an outside side surface (not visible in these views) facing away from the inside side surface 212b, 213b, and a lower surface (not visible in these views) facing away from the upper surface 212a, 213a.

In some embodiments, each hand controller 201, 202 includes a mounting assembly 215, 216, respectively. The mounting assembly 215, 216 may be used to attach, either directly or indirectly, the respective hand controller 201, 202 to a surgeon console of a surgical robotic system. In some embodiments, the mounting assembly 215 defines holes 217, which may be countersunk holes, configured to receive a screw or bolt to connect the left hand controller 201 to a surgeon console.

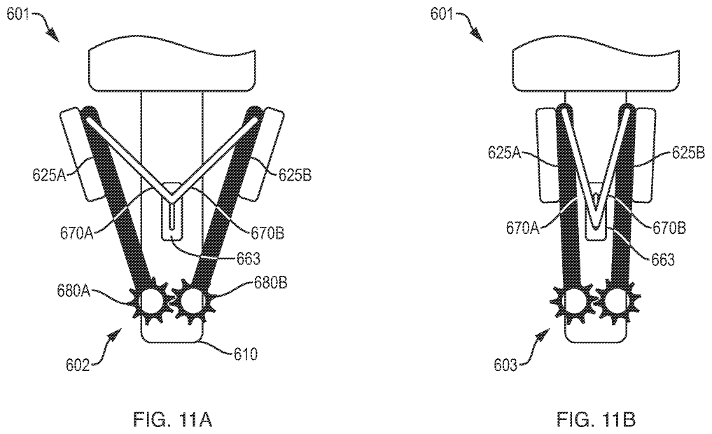

In some embodiments of the present disclosure, such as that depicted in FIGS. 6A and 6B, the hand controller includes two paddles, three buttons, and one touch input device. As will be explained herein, embodiments may feature other combinations of touch input devices, buttons, and levers, or a subset thereof. The embodiment shown as the left hand controller 201 features a first paddle 221 and a second paddle 222. Similarly, right hand controller 202 includes a first paddle 223 and a second paddle 224. In some embodiments, first paddle 221 is engaged with the second paddle 222 via one or more gears (not shown) so that a user depressing the first paddle 221 causes a reciprocal movement in the second paddle 222 and vice versa. Further description regarding a geared engagement between a first paddle and a second paddle is provided below with respect to FIGS. 11A and 11B. In another embodiment, first paddle 221 and second paddle 222 may be configured to operate independently. In embodiments employing reciprocal movement of the first and second paddle, a hand controller may employ only one signal indicating a deflection of the first paddle and the second paddle. In embodiments in which the first paddle and second paddle operate independently, a hand controller may employ a first signal indicating a deflection of the first paddle and a second signal indicating a deflection of the second paddle.

In some embodiments, the first paddle 221, 223 and the second paddle 222, 224 may be contoured to receive a thumb and/or finger of a user. In some embodiments, the first paddle 221, 223 extends from or extends beyond the outside side surface of the respective contoured housing 210, 211 the second paddle 222, 224 extends from or extends beyond the inside side surface 212b, 213b of the respective contoured housing. For each hand controller 210, 211, deflection or depression of the first paddle 221, 223, and the second paddle 222, 224, is configured to produce a signal that the surgical robotic system uses as an input to control a tool or an instrument tip (e.g., opening/closing an aperture of graspers/jaws of an instrument tip) at a distal end of a robotic arm of the surgical robotic system. For example, depressing first paddle and the second paddle may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, a housing of a hand controller may be contoured. For example, in FIGS. 6A and 6B, the contoured housing 210, 211 includes a rounded shape. In some embodiments, a housing may be shaped to have a contour to match a contour of at least a portion of a thumb of a user's hand. In some embodiments, the contoured housing 210, 211, the first paddle 221, 223, and the second paddle 222, 224, may each be shaped to comfortably and ergonomically receive a respective hand of a user. In some embodiments, a housing of the hand controller, a lever or levers of a hand controller, buttons of a hand controller and/or one or more touch input devices may have shapes and/or positions on the hand controller for fitting different palm sizes and finger lengths.

Left hand controller 201 also includes a first button 231, a second button 232, and a third button 233. Similarly, right hand controller 202 also includes a first button 234, a second button 235 and a third button 236. As will be described herein, each button may provide one or more inputs that may be mapped to a variety of different functions of the surgical robotic device to control the surgical robotic system including a camera assembly and a robotic arm assembly. In an embodiment, input received via the first button 231 of the left hand controller 201 and input received via the first button 234 of the right hand controller 202 may control a clutch feature. For example, by engaging the first button 231, 234 a clutch is activated enabling movement of the respective left hand controller 201 or right hand controller 20, by the operator without causing any movement of a robotic assembly (e.g., a first robotic arm, a second robotic arm, and a camera assembly) of the respective rob of the surgical robotic system. When the clutch is activated for a hand controller, movement of the respective right hand controller or left hand controller is not translated to movement of the robotic assembly. In some embodiments, an operator engaging a hand controller input (e.g., tapping or pressing a button) activates the clutch and the operator engaging again (e.g., tapping or pressing the button again) turns off the clutch or exits a clutch mode. In some embodiments, an operator engaging a hand controller input (e.g., tapping or pressing a button and holding the button) activates the clutch and the clutch stays active for as long as the input is active and exits the clutch when the operator is no longer engaging the hand controller input (e.g., releasing the button). Activating the clutch or entering the clutch mode for a hand controller enables the operator to reposition the respective hand controller (e.g., re-position the left controller 201 within the range of motion of the left hand controller 201 and/or re-position the right hand controller 202 within a range of motion of the right hand controller 202) without causing movement of the robotic assembly itself.

The second button 232 of the left hand controller 201 may provide an input that controls a pivot function of the surgical robotic device. An operator engaging (e.g., pressing and holding) the second button 232 of the left hand controller 201 may engage a pivot function or a pivot mode that reorients the robot assembly chest to center the camera on the midpoint between the instrument tips. The pivot function can be activated with a brief tap or held down to continuously track the instrument tips as they move, in accordance with some embodiments.

The second button 235 of the right hand controller 202 may provide input for entering a menu mode in which a menu is displayed on a graphical user interface of the surgical robotic system and exiting a menu mode. The operator may activate a menu mode by pressing the second button 235 a first time and disengage the menu function by pressing the second button 235 a second time. The operator may be able to select options within the menu by navigating the menu using the left hand controller and/or the right hand controller when the menu mode is engaged. For example, the first touch input device 242 of the right hand controller 202 may be used to navigate the menu and to select a menu item in some embodiments. While in a menu mode, movement of the robotic in response to movement of the left hand controller 201 or the right hand controller 202 may be suspended.

The third button 233 of the left hand controller and the third button 236 of the right hand controller may provide an input that engages or disengages an instrument control mode of the surgical robotic system in some embodiments. A movement of at least one of the one or more hand controllers when in the instrument mode causes a corresponding movement in a corresponding robotic arm of the robotic assembly. The instrument control mode will be described in more detail below.

The left hand controller 201 further includes a touch input device 241. Similarly, the right hand controller 202 further includes a touch input device 242. In an embodiment, the touch input device 241, 242 may be a scroll wheel, as shown in FIGS. 6A and 6B. Other touch input devices that may be employed include, but are not limited to, rocker buttons, joy sticks, pointing sticks, touch pads, track balls, trackpoint nubs, etc.

The touch input device 241, 242 may be able to receive input through several different forms of engagement by the operator. For example, where the touch input device 241, 242 is a scroll wheel, the operator may be able to push or click the first touch input device 241, 242, scroll the first touch input device 241, 242 backward or forward, or both.

In some embodiments, scrolling the first touch input device 241 of the left hand controller 241 forward may activate a zoom in function to magnify a view provided by the camera assembly of the surgical robotic system and displayed to the operator, and scrolling backward with first touch input device 241 may provide a zoom out function to reduce the view provided by the camera assembly of the surgical robotic device and displayed to the operator, or vice versa. In embodiments, the zoom function may be mechanical or digital. In some embodiments, the zoom function may be mechanical in part and digital in part (e.g., a mechanical zoom over one zoom range, and a mechanical zoom plus a digital zoom over another zoom range).

In some embodiments, clicking or depressing first touch input device 241 may engage a scan mode of the surgical robotic system. When in a scan mode, a movement of at least one of the left hand controller 201 or the right hand controller 202 causes a corresponding change in an orientation of a camera assembly of the robotic assembly without changing a position or orientation of either robotic arm of the surgical robotic system. In another embodiment, pressing and holding the first touch input device 241 may activate the scan mode and releasing the first touch input device 241 may end the scan mode of the surgical robotic system. In some embodiments, releasing the scan mode returns the camera to the orientation it was in upon entering the scan mode. In some embodiments, a function may be provided for locking the orientation upon exiting the scan mode (e.g., to change the “horizon” line).

In some embodiment, when in a menu mode and a left elbow menu item is selected, the first touch input device 241 of the left hand controller 201 may be used for selection of a direction and degree of left elbow bias. As used herein, elbow bias refers to the extent by which the virtual elbow of the robotic arm is above or below a neutral or default position.

In some embodiments, when in a menu mode, an operator may be able to select options within the menu by navigating the menu using the left hand controller and/or the right hand controller. For example, when in a menu mode, the touch input device 242 (e.g., scroll wheel) of the right hand controller provide a set of inputs for traversing a displayed menu and selecting an item in a displayed menu. For example, by scrolling forward on touch input device 242 the operator may move up the menu and by scrolling backwards with touch input device 242 the user may move down the menu, or vice versa. In an embodiment, by clicking first touch input device 242 the operator may make a selection within a menu.

In some embodiments, the touch input device 242 of the right hand controller 202 may be used to control right elbow bias when a right elbow bias menu item has been selected.

Functions of various buttons and the touch input device described above with respect to the left hand controller above may instead be assigned to the right hand controller, and functions of various buttons and the touch input device described above with respect to the right hand controller may instead be assigned to the left hand controller in some embodiments.

FIG. 6A also shows a schematic depiction 203 of a first foot pedal 251 and second foot pedal 252 for receiving operator input. As shown in FIG. 6A, in some embodiments the first foot pedal 251 engages a camera control mode of the surgical robotic system and the second foot pedal 252 engages a travel control mode of the surgical robotic system.

In some embodiments, when the camera control mode is activated e.g., using the foot pedal 251, movement of the left hand controller 201 and/or the right hand controller 202 by the operator may provide input that is interpreted by the system to control a movement of and an orientation of a camera assembly of the surgical robotic system while keeping positions of instrument tips of robotic arms of the robotic assembly constant.

In some embodiments, when the travel control mode is activated e.g., using the foot pedal 252, the left hand controller 201 and the right hand controller 202 may be used to move the robotic arm assembly of the surgical robotic system in a manner in which distal tips of the robotic arms direct or lead movement of a chest of the robotic assembly through an internal body cavity. In the travel control mode, a position and orientation of the camera assembly is automatically adjusted to maintain the view of the camera assembly directed at the tips (e.g., at a point between a tip or tips of a distal end of the first robotic arm and a tip or tips of a distal end of the second robotic arm). This may be described as the camera assembly being pinned to the chest of the robotic assembly and automatically following the tips. Further detail regarding the travel control mode is provided below.

In different embodiments, different functions may map to different buttons and different touch input devices of hand controllers. In different embodiments, different or other functions may corresponding to buttons and touch input devices of hand controllers that have a same physical structure.

For example, in some embodiments, compared with FIGS. 6A, 6B, the first button 231, 234 can provide an input that engages or disengages an instrument control mode of the surgical robotic system. In some embodiments, hand controller 201, 202 may not include an engage/disengage button. Instead, an operator may put his/her head close to a display such that his/her head may enter a surgeon vigilance sensor range, the operator can squeeze the paddles 221-224 to engage/disengage. In some embodiments, compared with FIGS. 6A, 6B, the second button 232 can engage a camera control mode. When the camera control mode is activated, movement of the left hand controller 201 and/or the right hand controller 202 by the operator may provide input that is interpreted by the system to control a movement of and an orientation of a camera assembly of the surgical robotic system while keeping positions of instrument tips of robotic arms of the robotic assembly constant. In some embodiments, the first pedal 251 can engage a pivot mode. In some embodiments, the functionality of the pivot mode may be consolidated into the camera mode and the system does not need to have a pivot mode. In some embodiments, compared with FIGS. 6A, 6B, the first pedal 252 can engage a translate mode.

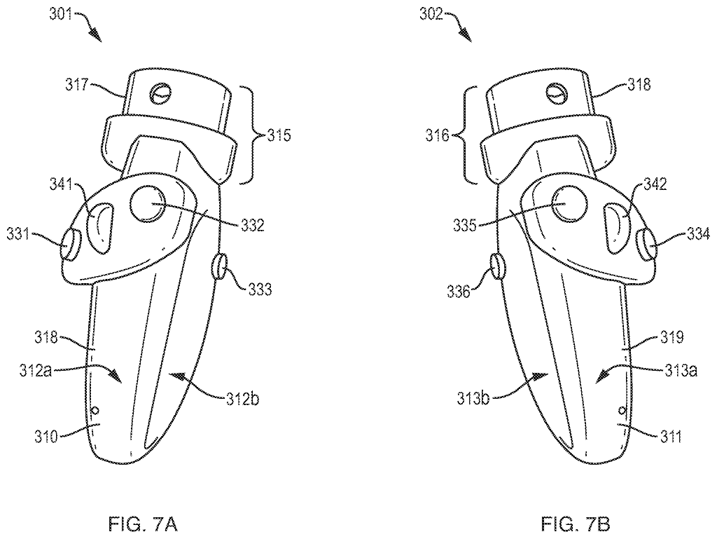

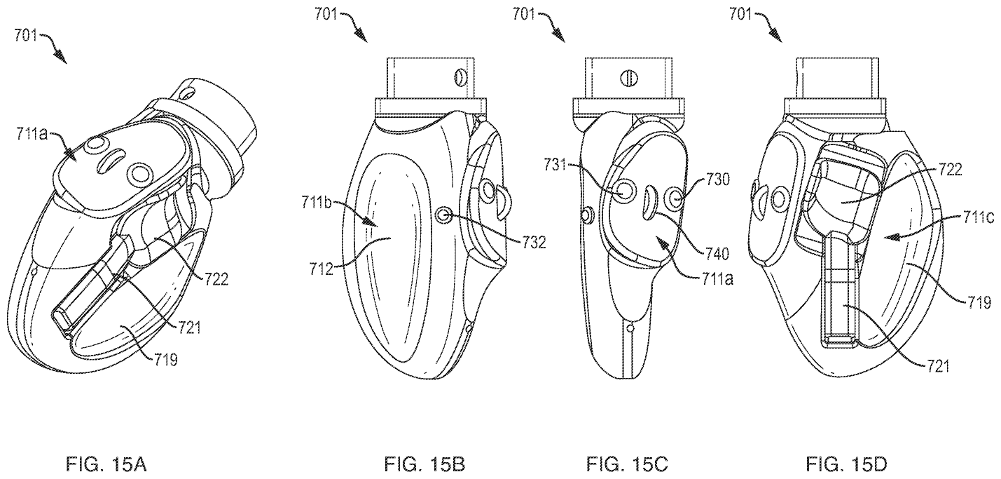

FIGS. 7A and 7B depict another embodiment according to the present disclosure featuring a left hand controller 301 and a right hand controller 302. The left hand controller 301 includes a contoured housing 310, and the right hand controller 302 includes a contoured housing 311. Each contoured housing 310, 311, includes an upper surface 312a, 313a, an inside side surface 312b, 313b adjacent the upper surface, an outside side surface (not visible in these views) facing away from the inside side surface 312b, 313b, and a lower surface (not visible in these views) facing away from the upper surface 312a, 313a (see outside side surface and outside side view of similar hand controller 701 in FIGS. 15-15D).

In some embodiments, the left hand controller 301 also includes a mounting assembly 315 including a proximal hand controller attachment 317, and the right hand controller 302 also includes a mounting assembly 316, including a proximal hand controller attachment 318. For each hand controller 301, 302, the respective mounting assembly 315, 316 may be used to attach the hand controller 301, 302 to a surgeon console of the surgical robotic system.

Each of the left hand controller 301 and the right hand controller 302 also includes a first button 331, 334, a second button 332, 335, and a third button 333, 336, respectively. In some embodiments, each of the first hand controller 301 and the second hand controller 301 also includes a touch input device 341, 342, respectively, which may be a scroll wheel. In each hand controller 301, 302, the first button 321, 334, the second button 332, 335, and the touch input device 341, 342 are disposed on or at an upper surface 312a, 313a of the housing. In some embodiments, the first button 321, 334, the second button 332, 335, and the touch input device 341, 342 are disposed on or at a portion of the upper surface 312a, 313a that projects from the upper surface. In each hand controller 301, 302, the third button 333, 336 is disposed on or at an inside side surface 312b, 313b of the housing. For each hand controller 301, 302, a lever (not visible in this view) extends from the respective outside side surface (not visible in this view). In some embodiments, a different mechanism may be used for a grasping input on a hand controller. For example, in some embodiments a hand controller may include a least one “pistol trigger” type button that can be pulled back to close and released to open instead of or in addition to a lever or levers.

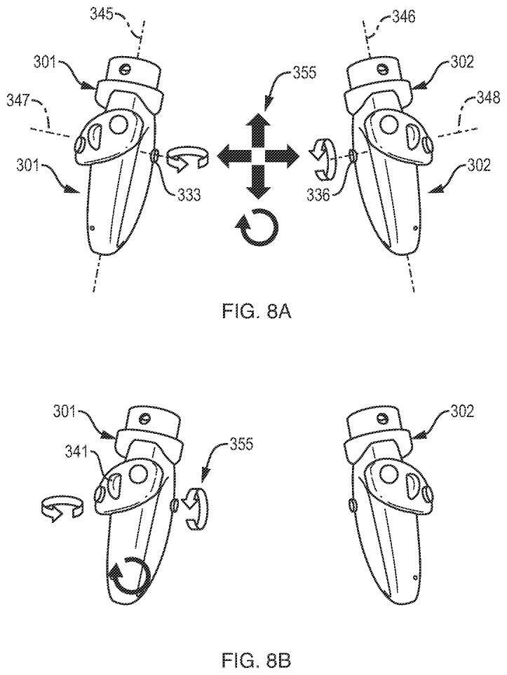

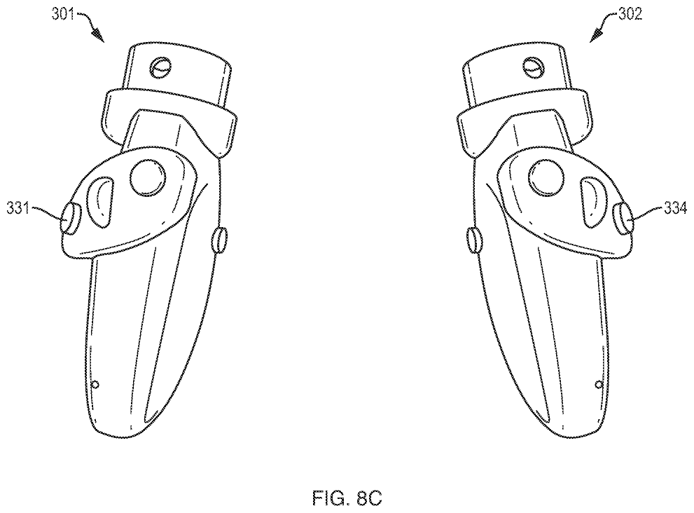

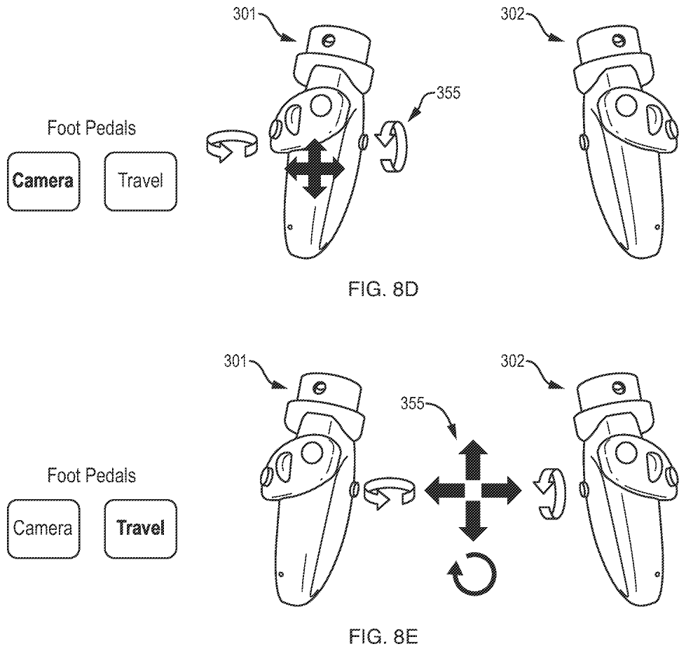

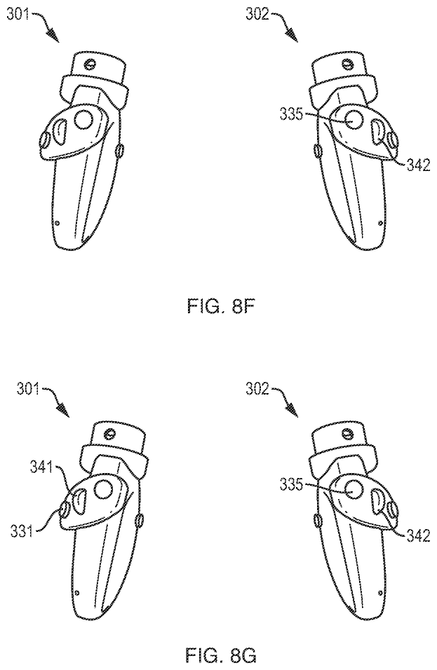

The contoured housing 310, 311 may be configured to comfortably and ergonomically mate with a corresponding hand of the operator. The operator may engage with the respective hand controller 301, 302 by placing the thumb of the respective hand on the inside side surface 312b, 313b, positioning the pointer finger or middle finger of the respective hand on or over the projecting portion of the upper surface 313a, 313a on which the first button 321, 334, the second button 332, 335, and the touch input device 341, 342 are disposed, and by positioning at least, the middle finger or ring finger of the respective hand on or over the outside side surface or the lever. An example of functionality that may be controlled by the first button 331, 334 the second button 332, 335 the third button 333, 336 and the touch input device 341, 342 will be further explained below with reference to FIGS. 8A to 8G.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may assigned to some buttons and some touch input devices in some embodiments. One of ordinary skill of the art in view of the present disclosure will also appreciate that some embodiments may not assign some of the functions described herein or any of the functions described herein to any of the buttons and/or touch input devices of hand controllers. In some embodiments, one or more functions may be assigned to a foot pedal of a surgical robotic system that includes one or more hand controllers as described herein.

By way of example, a set of functions that may be controlled by the left hand controller 301 and the right hand controller 302 for some embodiments of the present technology will now be described. In some embodiments, more frequently used functions, such as clutch, may be assigned to an button or touch input device that is easier for an operator to reach, which may be a first button 331 for some operators. Which button or touch input device is easier for an operator to reach may depend on a particular design of the hand controller, and may depend on a length of a digit (e.g., finger or thumb) of the operator and a palm size of the operator. In some embodiments, engaging (e.g., pressing or pressing and holding) the first button 331 of the left hand controller 301 may produce an input signal for control of a clutch function of the left hand controller 301. Pressing the second button 332 of the left hand controller may produce an input signal for controlling a reset feature of the surgical robotic system, which may also be described herein as a realign feature of the surgical robotic system. In some embodiments, the reset feature or realign feature causes the robotic arms and chest to change to a neutral or default configuration while maintaining locations of tips of end effectors or instruments of the robotic arms. Pressing the third button 333 of the left hand controller 301 may produce a signal for engaging/disengaging an instrument control mode of the surgical robotic system in some embodiments. Scrolling the touch input device 341 may produce a signal used to control a zoom function of the surgical robotic system. Pressing or pressing and holding the touch input device 341 may produce a signal used to activate a scan function or scan mode. Scrolling the touch input device 341 may produce a signal used to adjust left elbow bias when an elbow bias function is activated using the menu.

For right hand controller 302, pressing or pressing and holding the first button 334 may produce a signal used to control a clutch function for the right hand controller of the surgical robotic system. Pressing the second button 335 may produce a signal used to turn on and off a menu of the surgical robotic system. Pressing or pressing and holding the third button 336 of the right hand controller may produce a signal to engage or disengage an instrument control mode of the surgical robotic system. Scrolling the touch input device 342 of the right hand controller may produce a signal used traverse a menu or highlight a portion of the menu when the menu is displayed or the menu mode is active. Pressing the touch input device 342 may produce a signal used to select a highlighted portion or of the menu or feature on the menu when the menu is displayed. Scrolling the touch input device 342 may produce a signal used to select right elbow bias when the elbow bias function is activated using the menu.

Some functions and operations of the hand controllers are described with respect to FIGS. 8A to 8G. The left hand controller 301 and right hand controller 302 depicted in each of FIGS. 8A to 8G may be substantially similar to the right hand controller 301 and the right hand controller 302 shown in FIGS. 7A, 7B. Where like features are described, the same reference numerals are used.

FIG. 8A shows the left hand controller 301, the right hand controller 302, and third buttons 333, 336 of the left hand controller 301 and the right hand controller 302, respectively. In an embodiment, third buttons 333, 336 may each produce a signal to engage/disengage an instrument control mode of the surgical robotic system. When the instrument control mode is disengaged, input from the surgeon console via hand controllers does not result in any movement or other activity by the robotic assembly. Disengage does not affect the camera view but it does prevent control of any robotic movement (camera, scan, instruments, travel/tracking modes included). Unlike a clutch, “engage” input must occur to re-engage whereas clutch only has to be released to exit clutch function in accordance with some embodiments.

In some embodiments, the instrument control mode is a default control mode of the surgical robotic system. In the instrument control mode, motion of each hand controller corresponds to motion of an end effector of the respective robotic arm, but a chest or base of the robotic arms is not translated in space.

In the instrument control mode, the hand controllers are also used to operate some tool functions robotic arms of the surgical robotic assembly such as opening and closing graspers, or operating an electronic functionality of the surgical robotic system. The instrument control mode is also used to operate instruments attached to the arms of a surgical robotic device, for example, to manipulate tissue to cauterize tissue, to suture, or to perform other functions. In the instrument control mode, the instrument tips of the left robotic arm are controlled, at least in part, by the left hand controller 301 and the instrument tip or tips of the right robotic arm are controlled, at least in part, by the right hand controller 302. In some embodiments, a pedal assembly may include additional pedals to control some aspects of the instrument tips, such as an electrocautery function. In some embodiments, in an instrument control mode, an orientation and a position of the camera assembly remains fixed while the instrument tips are manipulated. In an embodiment, left hand controller 301 may be used to control motion of an instrument on the left arm assembly and right hand controller 302 may be used to control an instrument on the right arm assembly. For example, instruments may include graspers, scissors, cauteries, needles and other end effectors. In an embodiment, the instrument control mode may be activated by clicking an engage button (e.g. the third button 333 or the third button 336). In an embodiment, instrument control mode may be the default mode, such that when a user exits any other control mode while the system is engaged, the system returns to the instrument control mode. The left hand controller 301 and the right hand controller 302 may move independently. For example, either or both of the left hand controller 301 and the right hand controller 302 may be moved right, left, forward, backward, up, down, or any combination of those directions. Additionally, the left hand controller 301 and the right hand controller 302 may roll in a clockwise or counterclockwise direction about a longitudinal axis 345, 346 of the respective hand controller, may tilt/pitch forward or backward about a pitch axis of the respective hand controller, may rotate or tilt about a yaw axis, perpendicular to the longitudinal axis 345, 346 and to the pitch axis 347, 348 of the respective hand, or may do any combination of the aforementioned.

FIG. 8B depicts an embodiment in which the left hand controller 301 and, in particular, the touch input device 341 is used to select a scan mode. In embodiments, by pressing and holding the touch input device 341, the user may activate the scan mode. In the scan mode the user may change an orientation of the camera assembly of the surgical robotic system (e.g., yaw laterally and pitch up and down) while the arms and instruments remain fixed so that the user may, e.g., survey the abdomen, check the elbow position, or look for surgical materials. In the scan mode, movement of the left hand controller 301 may be used to control yaw, pitch, and roll of the camera assembly. In some embodiments, the operator exits the scan mode by releasing the touch input device 341. In some embodiments, after the user clicks the touch input device 341 a second time, the surgical robotic system will exit the scan mode. In some embodiments, when the scan mode is exited, an orientation of the camera assembly returns to the orientation it had when scan mode was activated.

FIG. 8C depicts a clutch function for a hand controller engaged by selecting either the first button 331 or the second button 335 of the hand controller in accordance with some embodiments. In an embodiment, the operator may engage (e.g., press or hold) the first button 331, 334 of the respective hand controller to activate the clutch with respect to that hand controller. In some embodiments, when the clutch is activated, the robotic surgical system remains in a current control mode, but disregards input from movement of the respective hand controller for which clutch has been engaged so that the user may re-position the respective left hand controller 301, or right hand controller 302, or within the range of motion of the respective controller. When the clutch is no longer activated or engaged, the surgical robotic system still remains in the same current control mode, but movement of the hand controllers causes the usual movements of the robotic assembly and the camera assembly corresponding to the current control mode. In some embodiments, a hand controller or a foot pedal may receive input for a universal clutch that functions as a clutch for both hand controllers collectively.

FIG. 8D depicts activating a camera control mode, using a foot pedal of the operator console to select the camera control mode. After the camera control mode has been selected, left hand controller 301 may be used to change an orientation (e.g., pitch, and yaw) and a position of the camera assembly of the robotic assembly of the surgical robotic system. In the camera control mode, the user may control the yaw, pitch, and roll of the camera assembly and may translate or displace the camera assembly up, down, forward, backward, right, left or any combination of the aforementioned. In the camera control mode, a chest of robotic arm assembly and at least a portion of each robotic arm may move, but an instrument tip or tips of each robotic arm remains stationary. Each of these movements of the camera assembly may be controlled with a corresponding movement of left hand controller 301, as shown by the movement indicator. In some embodiments, pressing the camera control mode pedal again causes the surgical robotic system to exit the camera control mode.

In some embodiments, after exiting the camera control mode, the framing selected in the camera control mode is maintained meaning that the camera assembly remains in a current position when returning to the instrument control mode and a view from the camera assembly remains the same when returning to the instrument control mode. The scan mode and the camera control mode may differ, at least, in that the camera control mode view is subsequently maintained in the instrument control mode, whereas the in the scan mode or with the scan function, a current view from the camera assembly is not maintained after the system returns to the instrument control mode. Further, the scan view does not enable translation of the camera assembly or movement of the chest of the robotic arm assembly. The scan mode enables the operator to view other portions of the interior cavity without moving the robotic arms before returning to a preferred or prior camera view using the scan function. The camera control mode enables an operator to reframe the camera view or change a perspective with respect to the instrument tips before returning to the instrument control mode or changing to a travel mode. In some embodiments, when in the camera control mode, the robotic surgical system may not recognize input from the right hand controller 302 when the left hand controller is being used for camera control, or the robotic surgical system may not recognize input from the left hand controller 301 when the right hand controller 302 is used for camera control. In other embodiments, when in the camera control mode, input from both hand controllers may be used.

FIG. 8E depicts hand controllers in a travel control mode. In some embodiments, the travel control mode may be selected by pressing a foot pedal. In some embodiments, the foot pedal may be pressed once to engage the travel control mode and a second time to disengage the travel control mode. Alternatively, in some embodiments, the travel control mode may be engaged while the pedal is depressed and may be disengaged when the pedal is released. Some embodiments may provide both functionalities by recognizing when the user briefly presses and releases the pedal and engaging the functionality or recognizing the user depressing the pedal for an extended period and engaging the function until the pedal is released.

When the travel control mode is engaged, movement of the left hand controller 301 and the right hand controller 302 are translated into corresponding movements of end effectors of the robotic arms of the robotic assembly. Similar to the instrument control mode, end effectors and tools of the robotic arms can be manipulated in the travel control mode. In the travel control mode, a view of the camera assembly tracks a midpoint between an instrument tip or tips of the right robotic arm and an instrument tip or tips of the left robotic arm. Unlike the instrument control mode, movement of the hand controllers can also cause displacement of a chest of the robotic assembly. The surgical robotic system establishes a cone of movement with respect to a displayed view from the camera assembly, when the instrument tips or end effectors are exceeding the cone of movement, the chest of the robotic system automatically moves. Further explanation of this aspect of the travel mode is provided below in the TravelMode section. In some embodiments, travel control mode may be used to navigate the robotic assembly to another location in an internal body cavity of a patient or to maintain visualization while a surgical task is performed. As noted above, in the travel mode, the camera assembly automatically tracks the midpoint between the instrument tips during movement of the instrument tips. Accordingly, travel mode may be useful for navigation and visualization for a task because the user will be able to maintain a consistent view of the instrument tip. This visualization may be valuable, for example, in procedures such as suturing the circumference of a mesh or creating a flap. As indicated by movement indicators 355, in the travel control mode both the left hand controller 301 and the right hand controller 302 may be moved independently or together and may move in the up, down, right, left, forward, backward, pitch, yaw and roll directions. In some embodiments, the movements of the hand controllers are translated to corresponding movements of the respective instrument tips of the robotic assembly and the robotic assembly automatically follows the instrument tips without requiring additional inputs from the user.

FIG. 8F depicts the second button 335 and touch input device 342 of the right hand controller 302 being used to control a menu function of the surgical robotic system. In an embodiment, the user may select the second button 335 to initiate the menu function which includes displaying a menu on a graphical user interface of the surgical robotic system, may scroll the touch input device 342 to move up or down the menu or to highlight menu items, and then may click the touch input device 342 in order to make a selection from the menu. In some embodiment, when the menu is engaged, an instrument control mode is disengaged or an instrument control mode, a travel control mode, and a camera control mode are all disengaged. In some embodiments, the second button 335 may be pressed or clicked once to engage the menu and pressed or clicked a second time to disengage the menu. In some other embodiments, the second button 335 may be held down to activate the menu option and released to deactivate the menu function.

FIG. 8G depicts the touch input device 341 of the left hand controller 301, the second button 335 of the right hand controller 302, and the touch input device 342 of the right hand controller 302 used to control an elbow bias functionality of the surgical robotic system. In an embodiment, the operator activates the menu function to display a menu, selects the elbow bias mode or function, and then scrolls the touch input device 341 up or down to and presses the touch input device 341 to select a bias for the left elbow of the surgical robotic system. Similarly, in an embodiment, the user may scroll touch input device 342 up or down and press the touch input device 342 to select a bias for the right elbow of the surgical robotic system. In some embodiments, options for elbow bias include one or more levels above a nominal or default elbow bias, and one or more levels below a normal or default elbow bias.

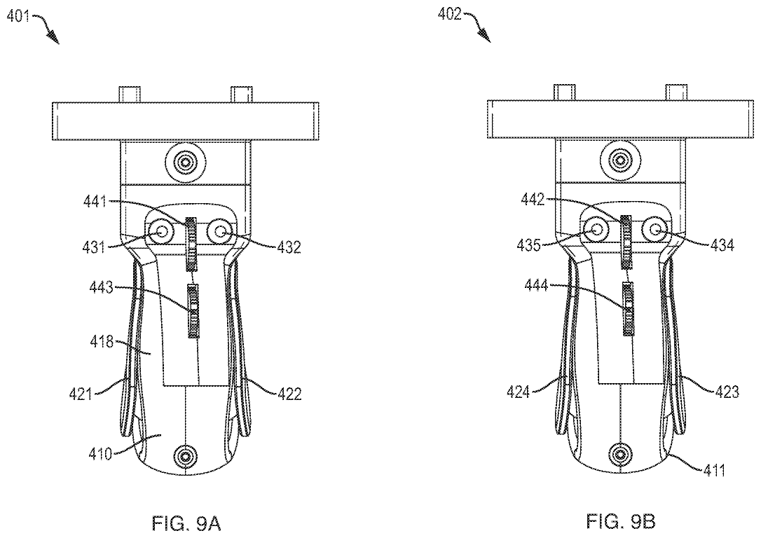

FIGS. 9A and 9B depict another embodiment of a left hand controller 401 and a right hand controller 402. Each of the hand controllers 401, 402 includes a contoured housing 410, 411, and a first button 431, 434 and a second button 432, 435, on a top surface of the housing. Each hand controller 401, 402 includes a first paddle or grasper 421, 422 and a second paddle or grasper 423, 424. Each hand controller 401, 402 also includes a first touch input device 441, 442 and a second touch input device 443, 444 (e.g., scroll wheels).

In some embodiments, the first button 431 of the left hand controller 401 is used to active the menu or close the menu and the first button 434 of the right hand controller 402 is used to open a right elbow bias menu. In some embodiments, the second button 432 of the left hand controller 401 is also used to active the menu or close the menu and the second button 435 of the right hand controller 402 is used to open a right elbow bias menu. In some embodiments, engaging an arm bias button on the left hand controller or an arm bias button on the right hand opens an arm bias menu or arm bias menus for both arms. In some embodiments, the first touch input device 441 of the first hand controller 401 is used for traversing a menu or highlighting menu items to be selected when the menu is displayed, and for selecting a left elbow bias when the left elbow menu is displayed. In some embodiments, the first touch input device 442 of the first hand controller 402 is used for traversing a menu or highlighting menu items to be selected when the menu is displayed, and for selecting a right elbow bias when the right elbow menu is displayed. In some embodiments, the second touch input device 443 of the left hand controller 401 is used to engage or disengage an instrument control mode when pressed or clicked, and used to set a configuration of the robotic arms and chest of the robotic assembly while keeping instrument stationary when the second touch input device 443 is rolled forward. In some embodiments, the second touch input device 444 of the second hand controller 402 is used to engage or disengage an instrument control mode when pressed or clicked.

In some embodiments, different functions may be assigned to some or all of the buttons and touch input devices of the left hand controller 401 and the right hand controller 402. For example, in some embodiments, the second button 432 of the left hand controller 401 is used to open or close the menu. In some embodiments, the second button 435 of the right hand controller 402 is used to open or close the menu. In some embodiments, the second button 432 is used to set a configuration of the robotic arms and chest of the robotic assembly while keeping instrument stationary when the second button 432 is rolled forward. In some embodiments, the first button 434 of the right hand controller is used to select a right elbow bias when an elbow bias menu is displayed and the second button 435 of the right hand controller 402 is used to the select a left elbow bias when an elbow bias menu is displayed. In some embodiments, the first touch input device 442 of the right hand controller 402 is used for traversing a menu or highlighting menu items to be selected when the menu is displayed and the second touch input device 443 of the left hand controller 401 is used for engaging or disengaging an instrument control mode when pressed, for engaging a clutch for the hand controller when slid or rolled backward, and for engaging a scan mode when slid or rolled forward. In some embodiments, rolling the first touch control input 442 of the right hand controller 402 may be used to highlight an elbow bias level for selection. In some embodiments, pressing the second touch control input 444 of the right hand controller 402 may be used to engage or disengage the instrument control mode and sliding or rolling second touch control input 444 may engage the clutch. In some embodiments, pressing a specific pedal while pressing or rolling the first touch control input 442 of the right hand controller 402 may change a corresponding function of pressing or rolling the first touch control input 442. For example in some embodiments, pressing and holding the first touch control input 442 while holding the specific pedal may activate a camera scan mode or camera scan feature. In some embodiments, clicking the first touch control input 442 while holding the specific pedal may cause a pose of the robotic assembly to reset. In some embodiments, rolling the first touch control input 442 forward or backward while holding the specific pedal may increase or decrease a zoom of an image from the camera assembly. In some embodiments, the foot pedal 203 can include a third pedal for a clutch function. The hand controllers 401, 402 may not include the clutch function.

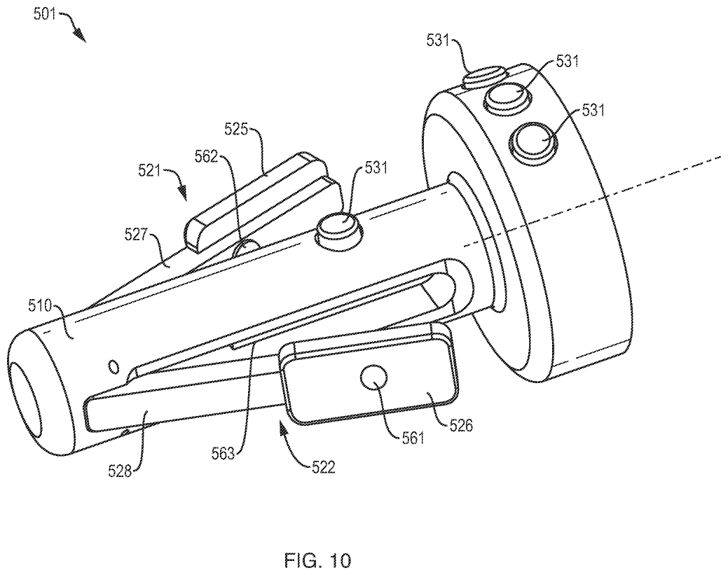

FIG. 10 includes a hand controller 501 according to another embodiment of the present technology. The hand controller 501 includes a contoured housing 510, a first paddle 521, a second paddle 522, and buttons 531. In some embodiments, the first paddle 521 and the second paddle 522 may be connected via a geared connection (not shown) such a movement of the first paddle 521 causes a reciprocal movement of the second paddle 522, and a movement of the second control 522 causes a reciprocal movement of the first paddle 521.

In some embodiments, the first paddle 521 includes a finger pad 525. In some embodiments the finger pad 525 forms at least part of a contact surface of the first paddle 521. In some embodiments, a magnet 562 is located on the first paddle 521 opposite the finger pad 525 and proximate to the contoured housing 510. In some embodiments, a printed circuit board (PCB) 563 is housed within the contoured housing 510 is located. In embodiments, PCB 563 may include or connect with one or more proximity and/or optical sensors. In some embodiments, PCB 563 may be configured to determine the distance between the magnet 562 and the PCB 563 or the proximity sensor or to produce a signal corresponding to the distance between the magnet 562 and the PCB 563 or proximity sensor. In some embodiments, the magnet 562 may instead be disposed on the second paddle. In some embodiments, a magnet may be disposed on the first paddle and another magnet may be disposed on the second paddle.

In some embodiments, at least one of the one or more hand controllers includes a sensor configured to sense contact of a hand of the operator with the hand controller or to sense proximity of a hand of the operator to the hand controller. In some embodiments, an input from the sensor is used for activating or exiting an instrument control mode of the surgical robotic system. In some embodiments, at least one sensor may be disposed on a lever of the hand controller. In some embodiments, at least one sensor may be disposed on a portion of the housing that is not the lever. In some embodiments, a hand controller may have multiple sensors for sensing engagement of a hand controller. In some embodiments, a sensor is optical. In some embodiments a sensor is a capacitive sensor. In some embodiments, another type of sensor may be employed.

In some embodiments, a surgical robotic system may employ a sensing system that does not include a sensor on a hand controller to sense engagement with the hand controller or engagement with the surgical robotic system (e.g., optical tracking of hands of an operator, optical tracking of a head of an operator, tracking eyes of an operator, etc.)

In some embodiments, the second paddle 522 includes a finger pad 526. In some embodiments the finger pad 526 forms at least part of a contact surface of the second paddle 522. In some embodiments, the second paddle 552 includes an optical window 561 and an optical sensor positioned to receive light incident on the optical window 561 (not shown). In some embodiments, the optical sensor produces a signal indicating that something (e.g., a portion of an operator's hand) is positioned over the optical window 561 blocking at least some incident light from reaching the optical window, or produce a signal indicating that nothing (e.g., no portion of an operator's hand) is positioned over the optical window 561 to block at least a portion of the incident light from reaching the optical window 561. Such a signal may be used to detect whether an operator's hand is positioned on the hand controller and disable at least some functions or the hand controller or disengage control modes of the hand controller when the operator's hand is not positioned on the hand controller. In some embodiments, a first hand controller may also or alternatively include such as sensor. In some embodiments, a capacitive sensor is used to enable engagement. Because a surgeon may have to remove their hands when throwing sutures, a capacitive sensor may not be used to disengage or disable functionality in some embodiments. In some embodiments, capacitive sensing of the fingertips could be used conjunction with another form of “vigilance” detection (e.g., eye tracking or optical sensors for the forehead) to allow engagement but then only the eye/forehead tracking would cause disengagement if the user did not have their eyes/head in the correct position.

FIGS. 11A and 11B schematically depicts a hand controller 601 with a different mechanism of measuring deflection of paddles in accordance with some embodiments. FIG. 11A depicts a hand controller 601 with a first paddle 625A and a second paddle 626B in a first position/orientation 602, and FIG. 11B depicts the hand controller 601 with the first paddle 625A and the second paddle 626B in a second depressed position/configuration 603. The first paddle 625A is connected to a housing or body 610 of the hand controller 601 via a first gear 680A at a proximal end of the first paddle and via an axle that engages first gear 680A. The second paddle 625B is connected to the housing or body 610 of the hand controller 601 via second gear 680B at a proximal end of the second paddle and via an axle that engages second gear 680B. Additionally, the first gear 680A and the second gear 680B engage each other such that motion of first paddle 625A causes a reciprocal motion of second paddle 625B and vice versa. Hand controller 601 also features a linear sensor 663. The first paddle 625A is connected to the linear sensor 663 via a first linkage 670A and the second paddle 625B is connected to the linear sensor 663 via a second linkage 670B. The linear sensor 663 is configured to determine the location at which the first linkage 670A and the second linkage 670B engage with the linear sensor 663 such that the linear sensor 663 for identification of a configuration of the first paddle 625A and the second paddle 625B (e.g., to determine how open or how closed the first paddle 625A and second paddle 625B are with respect to each other). In various embodiments, the linear sensor 670B may be a PCB with a linear sensor or another sensor configured to determine a location, such as an optical sensor or a proximity sensor.

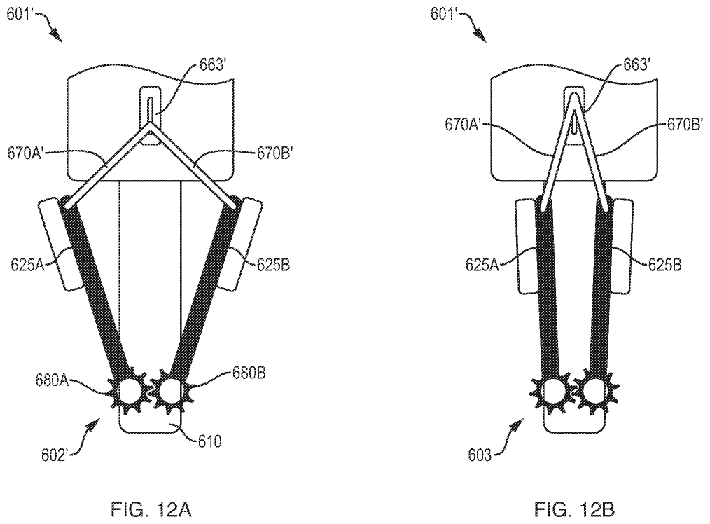

FIGS. 12A and 12B schematically depicts a hand controller 601′ similar to the hand controller 601 of FIGS. 11A and 11B with first and second paddles 625A and 625B in an open configuration (FIG. 12A) and in a more closed configuration (FIG. 12B). In hand controller 601′, a linear sensor 663′ is disposed further toward a distal end of the hand controller 601′ such that a first linkage 670A′ that connects with the linear sensor 663′ form an obtuse angle relative to the first paddle 625 and a second linkage 670B′ that connects with the linear sensor 663′ forms an obtuse angle relative to the second paddle 625B.

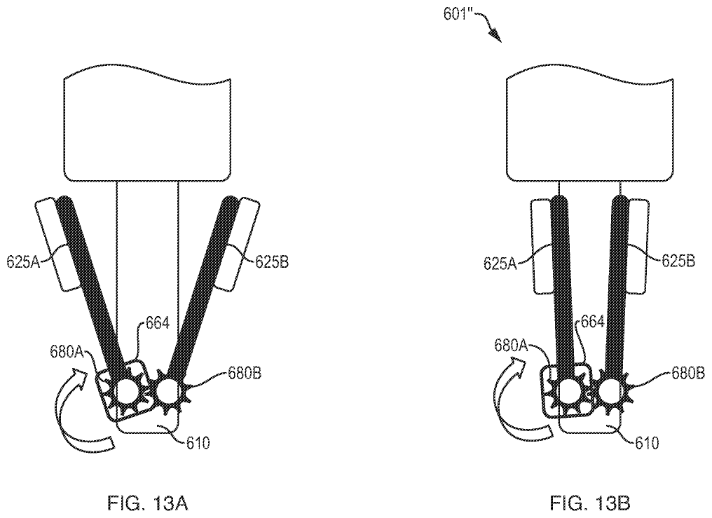

FIGS. 13A and 13B schematically depict a hand controller 601″ similar to the hand controller 601 of FIGS. 11A and 11B with first and second paddles 625A and 625B in an open configuration (FIG. 13A) and in a more closed configuration (FIG. 13B). In hand controller 601″, a rotary sensor 664 is engaged with the first gear 680A to measure an angular positon of the first paddle 625A which is related to a configuration of the first paddle 625A and the second paddle 625B (e.g., how open or how closed the first paddle 625A and second paddle 625B are with respect to each other). In some embodiments, the rotary sensor may be engaged with the second gear 680B instead of the first gear 680A.

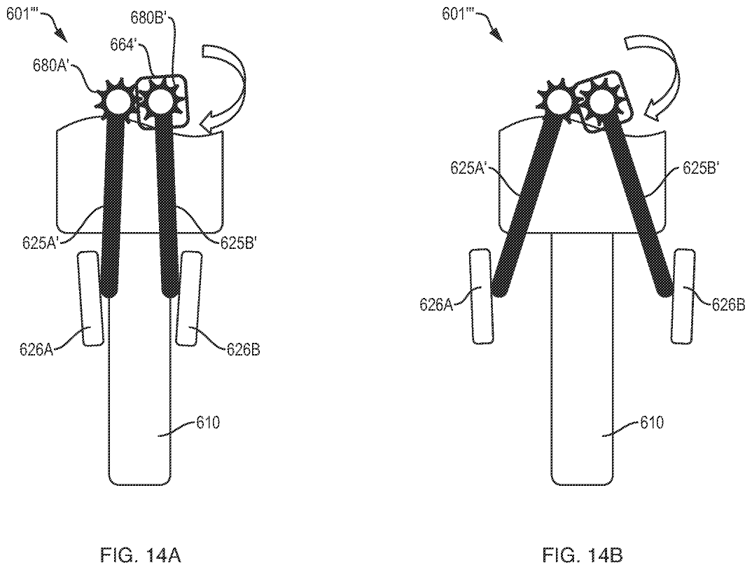

FIGS. 14A and 14B schematically depict a hand controller 601′″ similar to the hand controller 601″ of FIGS. 13A and 13B with first and second paddles 625A′ and 625B′ in an open configuration (FIG. 13A) and in a more closed configuration (FIG. 13B). In hand controller 601′″ first and second paddles 625A′ and 625B′ extend in a proximal direction and each of first and second paddles 625A′ and 625B′ have a respective gear 680′A and 680 B′ associated pivot point distal to a finger pad 626A, 626B of the respective paddle. Further, each finger pad 626A, 626B has a rotating or pivoting connection with the rest of its associated paddle 625A′, 625B′, respectively.

FIGS. 15A-15D depicts various views of another embodiment of a hand controller 701 of the present disclosure. The hand controller 701 includes a housing 710 having a top surface 711a, an inside side surface 711b, an outside side surface 711c, and a lower surface (not visible in these views). The hand controller 701 includes a thumb pad 712 on the inside side surface 711b, a paddle 721 with finger cup 722, and a finger pad 719 disposed at or extending from at the outside side surface 711c. The hand controller 701 also includes a first button 730, a second button 731 and a touch input device 740 disposed the top surface 711a. The hand controller 701 also includes a third button 731 disposed at the inside facing surface 711b. The hand controller 701 is a right hand controller. In use, the operator's thumb would normally be positioned on or about thumb pad 712, the user's index finger would be positioned near the first button 730, the second button 731 or the touch input device 740, the user's middle finger would be positioned on or over the finger cup 722 of the paddle 721, and the user's remaining fingers positioned on or near finger pad 719.

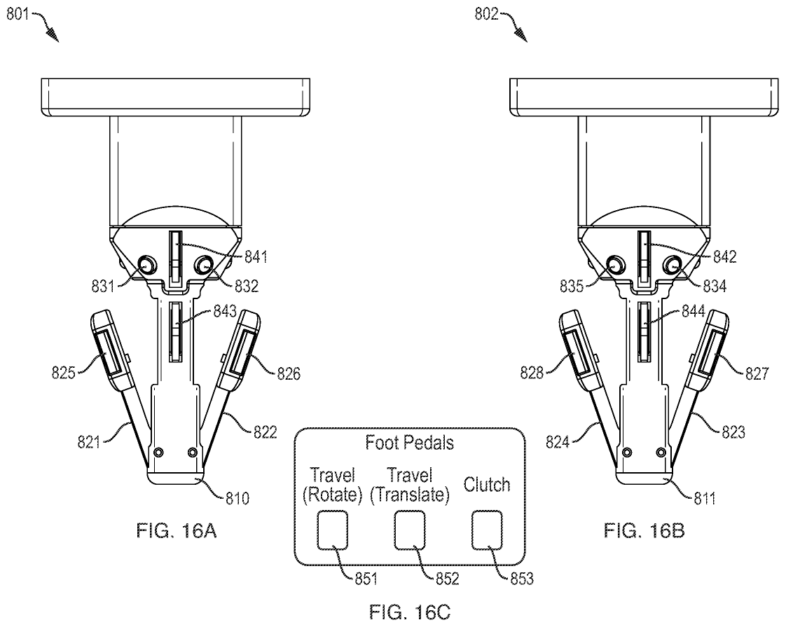

FIGS. 16A and 16B depict another embodiment of a left hand controller 801 and a right hand controller 802. FIG. 16C depict another embodiment of foot pedals.

Each of the hand controllers 801, 802 includes a housing 810, 811, a first paddle 821, 823 including a finger pad 825, 827, and a second paddle 822, 824, including a finger pad 826, 828, a first button 831, 834, a second button 832, 835, a first touch input device 841, 842, and a second touch input device 843, 844 (e.g., trackwheels, mini joysticks, touch pads, or any other suitable multi-function device).

In some embodiments, the first button 831 of the left hand controller 801 is used to active the menu or close the menu and the first button 834 of the right hand controller 802 is used to open a right elbow bias menu. In some embodiments, the second button 832 of the left hand controller 801 is also used to active the menu or close the menu and the second button 835 of the right hand controller 802 is used to open a right elbow bias menu. In some embodiments, engaging an arm bias button on the left hand controller or an arm bias button on the right hand opens an arm bias menu or arm bias menus for both arms. In some embodiments, the first touch input device 841 of the first hand controller 801 is used for traversing a menu or highlighting menu items to be selected when the menu is displayed, and for selecting a left elbow bias when the left elbow menu is displayed. In some embodiments, the first touch input device 842 of the first hand controller 802 is used for traversing a menu or highlighting menu items to be selected when the menu is displayed, and for selecting a right elbow bias when the right elbow menu is displayed. In some embodiments, the second touch input device 843 of the left hand controller 801 is used to engage or disengage an instrument control mode when pressed or clicked, and used to set a configuration of the robotic arms and chest of the robotic assembly while keeping instrument stationary when the second touch input device 843 is rolled forward. In some embodiments, the second touch input device 844 of the second hand controller 802 is used to engage or disengage an instrument control mode when pressed or clicked.

In some embodiments, different functions may be assigned to some or all of the buttons and touch input devices of the left hand controller 801 and the right hand controller 802. For example, in some embodiments, the second button 832 of the left hand controller 801 is used to open or close the menu. In some embodiments, the second button 835 of the right hand controller 802 is used to open or close the menu. In some embodiments, the second button 832 is used to set a configuration of the robotic arms and chest of the robotic assembly while keeping instrument stationary when the second button 832 is rolled forward. In some embodiments, the first button 834 of the right hand controller is used to select a right elbow bias when an elbow bias menu is displayed and the second button 835 of the right hand controller 802 is used to the select a left elbow bias when an elbow bias menu is displayed. In some embodiments, the first touch input device 842 of the right hand controller 802 is used for traversing a menu or highlighting menu items to be selected when the menu is displayed and the second touch input device 843 of the left hand controller 801 is used for engaging or disengaging an instrument control mode when pressed, for engaging a clutch for the hand controller when slid or rolled backward, and for engaging a scan mode when slid or rolled forward. In some embodiments, rolling the first touch control input 842 of the right hand controller 802 may be used to highlight an elbow bias level for selection. In some embodiments, pressing the second touch control input 844 of the right hand controller 802 may be used to engage or disengage the instrument control mode and sliding or rolling second touch control input 844 may engage the clutch. In some embodiments, pressing a specific pedal while pressing or rolling the first touch control input 842 of the right hand controller 802 may change a corresponding function of pressing or rolling the first touch control input 842. For example in some embodiments, pressing and holding the first touch control input 842 while holding the specific pedal may activate a camera scan mode or camera scan feature. In some embodiments, clicking the first touch control input 842 while holding the specific pedal may cause a pose of the robotic assembly to reset. In some embodiments, rolling the first touch control input 842 forward or backward while holding the specific pedal may increase or decrease a zoom of an image from the camera assembly. In some embodiments, the first foot pedal 851 can be used to engage a rotation function of a travel mode to rotate robotic arm assemblies and a camera assembly. The second foot pedal 852 can be used to engage a translation function of a travel mode to translate robotic arm assemblies and a camera assembly. The third food pedal 853 can be used to activate a clutch function. In some embodiments, the first, second and third foot pedals 851-853 can be used with other embodiments hand controllers (e.g., hand controllers 301, 302 illustrated in FIGS. 7A, 7B hand controllers 401, 402 illustrated in FIGS. 9A, 9B, or other hand controllers as taught herein). When the foot pedals include a clutch function, the hand controllers 801, 802 may not include the clutch function.

FIG. 17 schematically depicts a graphical user interface 900 including a camera view portion 910 displaying a view from the camera assembly, a first information portion 920, and a second information portion 930 that each identify a current control mode of the surgical robotic system.

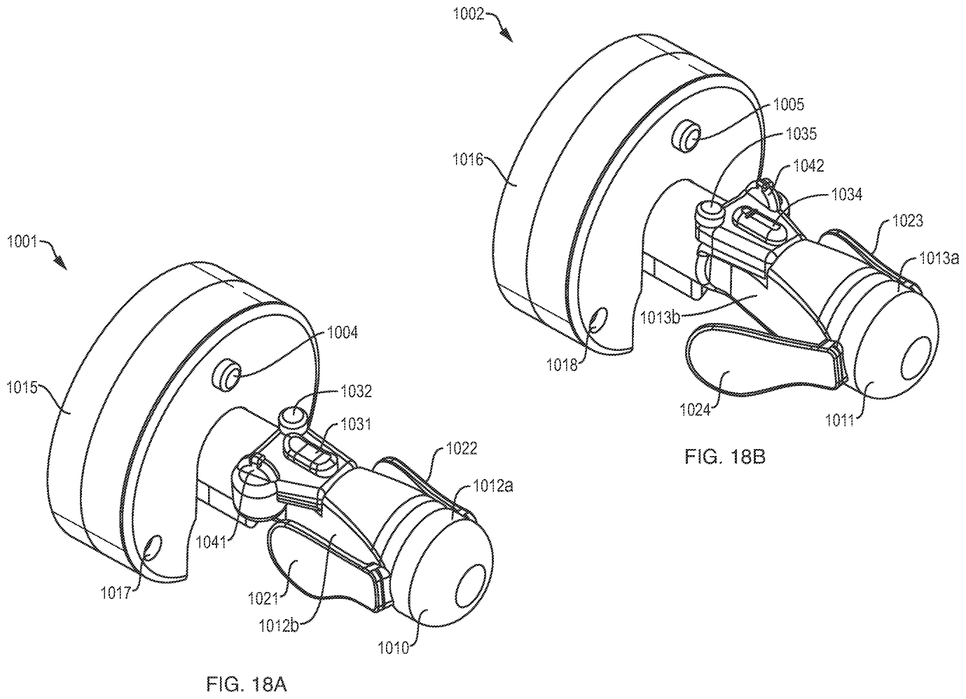

FIGS. 18A and 18B depict another embodiment according to the present disclosure featuring a left hand controller 1001 and a right hand controller 1002. The left hand controller 1001 includes a contoured housing 1010, and the right hand controller 1002 includes a contoured housing 1011. Each contoured housing 1010, 1011, includes an upper surface 1012a, 1013a, an inside side surface 1012b, 1013b adjacent the upper surface, an outside side surface (not visible in these views) facing away from the inside side surface 1012b, 1013b, and a lower surface (not visible in these views) facing away from the upper surface 1012a, 1013a (see outside side surface and outside side view of similar hand controller 701 in FIGS. 15-15D).

Each hand controller 1001, 1002 includes a mounting assembly 1015, 1016, respectively. The mounting assembly 1015, 1016 that may be used to attach, either directly or indirectly, each of the respective hand controllers 1001, 1002 to a surgeon console of a surgical robotic system. The mounting assembly 1015 includes an aperture 1017 and the mounting assembly 1016 defines an aperture 1018. The apertures 1017, 1018 may be countersunk apertures, configured to receive a screw or bolt to connect the respective hand controller 1001, 1002 to a surgeon console. The mounting assembly 1015 includes a button 1004 and the mounting assembly 1016 includes a button 1005. The buttons 1004, 1005 provide an input to toggle between insertion and extraction of one or more robotic arm assemblies 42A, 42B as well as the camera assembly 44. For example, the button 1004 can be used to insert or extract a first robotic arm 42A and the button 1005 can be used to insert or extract a second robotic arm 42B. In some embodiments, the buttons 1004, 1005 do not actually control insertion or extraction of the camera assembly 44, but allow an operator to enter a mode of insertion or extraction. The actual processes for the camera assembly 44 can be controlled by other user elements.

Each of the left hand controller 1001 and the right hand controller 1002 also includes a first button 1031, 1034, a second button 1032, 1035, a touch input device 1041, 1042 (e.g., a joy stick, or scroll wheel), respectively. In each hand controller 1001, 1002, the first button 1021, 1034, the second button 1032, 1035, and the touch input device 1041, 1042 are disposed on or at an upper surface 1012a, 1013a of the housing 1010, 1011, respectively. In some embodiments, the first button 1021, 1034, the second button 1032, 1035, and the touch input device 1041, 1042 are disposed on or at a portion of the upper surface 1012a, 1013a that projects from the upper surface. For each hand controller 1001, 1002, a lever (not visible in this view) extends from the respective outside side surface (not visible in this view). In some embodiments, a different mechanism may be used for a grasping input on a hand controller. For example, in some embodiments a hand controller may include a least one “pistol trigger” type button that can be pulled back to close and released to open instead of or in addition to a lever or levers.

The left hand controller 1001 includes a first paddle 1021 and a second paddle 1022. Similarly, right hand controller 1002 includes a first paddle 1023 and a second paddle 1024. In some embodiments, first paddle 1021, 1023 is engaged with the second paddle 1022, 1024 of each hand controller 1001, 1002 via one or more gears (not shown) so that a user depressing the first paddle 1021, 1023 causes a reciprocal movement in the second paddle 1022, 1024 and vice versa, respectively. Further description regarding a geared engagement between a first paddle and a second paddle is provided above with respect to FIGS. 11A and 11B. In another embodiment, the first paddle 1021, 1023 and the second paddle 1022, 1024 of each hand controller may be configured to operate independently. In embodiments employing reciprocal movement of the first and second paddles, the hand controller 1001, 1002 may employ some form of a signal or other indicator indicating a deflection of the first paddle 1021, 1023 and the second paddle 1022, 1024. In embodiments in which the first paddle and second paddle operate independently, the hand controller 1001, 1002 may employ a first signal or other indicator indicating a deflection of the first paddle 1021, 1023 and a second signal or other indicator indicating a deflection of the second paddle 1022, 1024.

In some embodiments, the first paddle 1021, 1023 and the second paddle 1022, 1024 may be contoured to receive a thumb and/or finger of a user. In some embodiments, the first paddle 1021, 1023 extends from or extends beyond the outside side surface of the respective contoured housing 1010, 1011 the second paddle 1022, 1024 extends from or extends beyond the inside side surface 1012b, 1013b of the respective contoured housing. For each hand controller 1010, 1011, deflection or depression of the first paddle 1021, 1023, and the second paddle 1022, 1024, is configured to trigger a signal that the surgical robotic system uses as an input to control a tool or an instrument tip (e.g., opening/closing an aperture of graspers/jaws of an instrument tip) at a distal end of a robotic arm of the surgical robotic system. For example, depressing first paddle 1021, 1023 and the second paddle 1022, 1024 may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

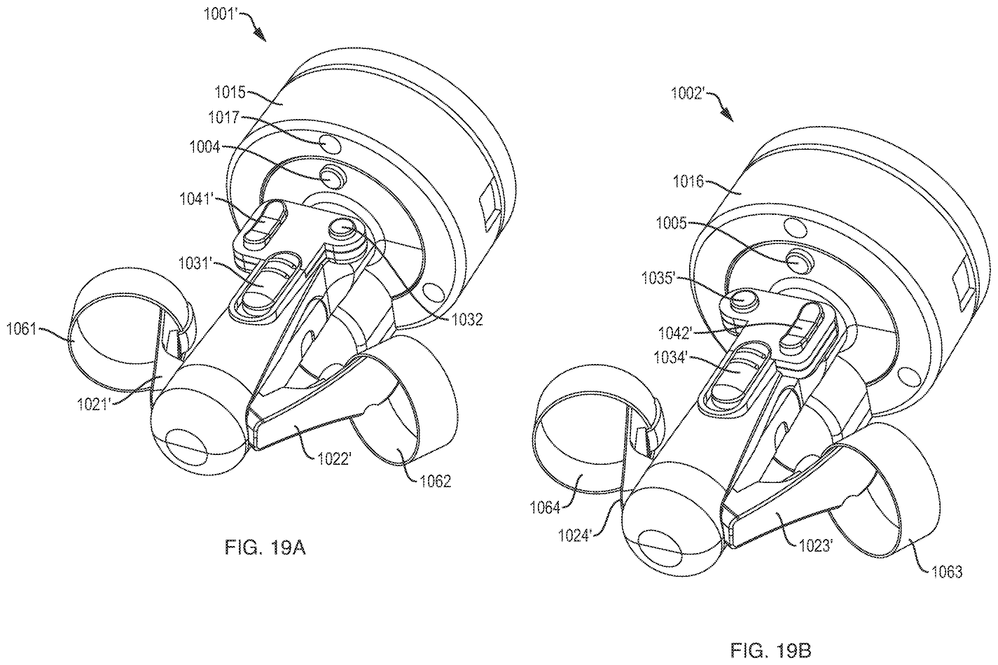

In some embodiments, each of the first paddle 1021, 1023 and the second paddle 1022, 1024 can have a loop to receive a thumb and/or finger of a user, as further described with respect to FIGS. 19A and 19B. In some embodiments, parameters (e.g., length, angle, finger ergonomics, and the like) of each of the first paddle 1021, 1023 and the second paddle 1022, 1024 can be adjusted.

The contoured housing 1010, 1011 may be configured to comfortably and ergonomically mate with a corresponding hand of the operator. The operator may engage with the respective hand controller 1001, 1002 by placing the thumb of the respective hand on the second paddle 1022, 1024, positioning the pointer finger or middle finger of the respective hand on or over the projecting portion of the upper surface 1013a, 1013a on which the first button 1021, 1034, the second button 1032, 1035, and the touch input device 1041, 1042 are disposed, and by positioning at least, the middle finger or ring finger of the respective hand on or over the first paddle 1021, 1024.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may be assigned to some buttons and some touch input devices in some embodiments. In some embodiments, one or more functions may be assigned to a foot pedal of a surgical robotic system that includes one or more hand controllers as described herein.

By way of example, a set of functions that may be controlled by the left hand controller 1001 and the right hand controller 1002 for some embodiments of the present technology will now be described.

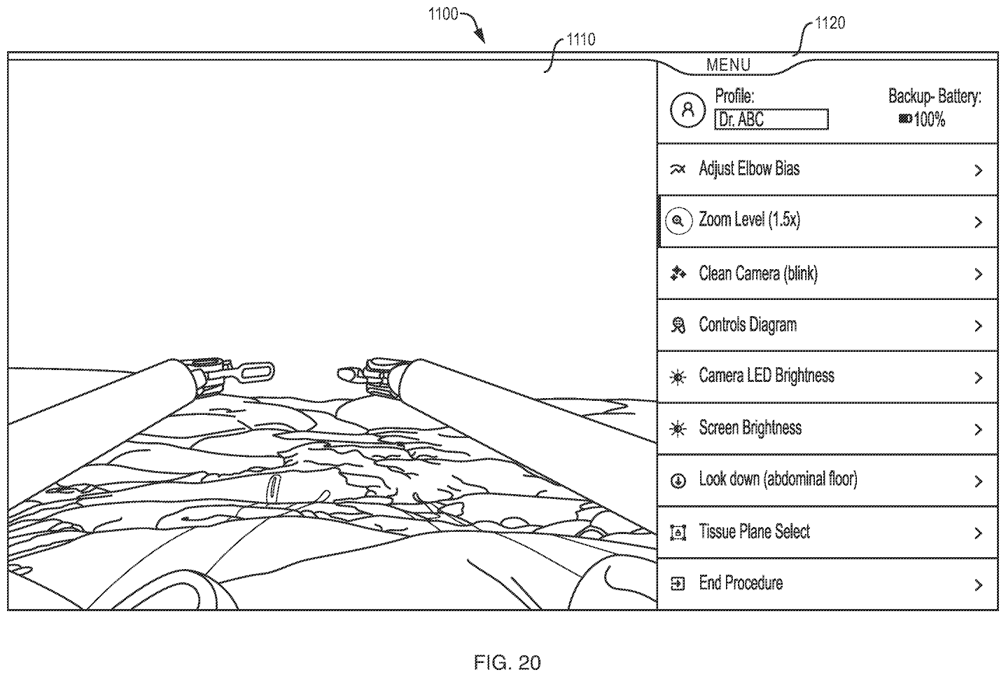

For left hand controller 1001, pressing or pressing and holding the first button 1004 may trigger a signal used to engage an insertion or extraction for a left robotic arm assembly and/or a camera assembly of the surgical robotic system. Pressing or pressing and holding the first button 1031 may trigger a signal used to control a clutch function for the left hand controller of the surgical robotic system. Pressing or pressing and holding the second button 1032 may trigger a signal used to engage or disengage a camera control mode of the surgical robotic system. Scrolling the touch input device 1041 forward may activate a zoom in function to magnify a view provided by the camera assembly of the surgical robotic system and displayed to the operator, and scrolling backward with first touch input device 1041 may provide a zoom out function to reduce the view provided by the camera assembly of the surgical robotic device and displayed to the operator, or vice versa. Scrolling the touch input device 1041 may trigger a signal used to select left elbow bias when an elbow bias function is activated using a menu (as illustrated in FIG. 20).

For right hand controller 1002, pressing or pressing and holding the first button 1005 may trigger a signal used to engage an insertion or extraction for a right robotic arm assembly and/or a camera assembly of the surgical robotic system. Pressing or pressing and holding the first button 1034 may trigger a signal used to control a clutch function for the right hand controller of the surgical robotic system. Clicking or depressing the second button 1035 may engage a scan mode of the surgical robotic system. When in a scan mode, a movement of at least one of the left hand controller 1001 or the right hand controller 1002 causes a corresponding change in an orientation of a camera assembly of the robotic assembly without changing a position or orientation of either robotic arm of the surgical robotic system. In another embodiment, pressing and holding the second button 1035 may activate the scan mode and releasing the second button 1035 may end the scan mode of the surgical robotic system. In some embodiments, releasing the scan mode returns the camera to the orientation it was in upon entering the scan mode. In some embodiments, a function may be provided for locking the orientation upon exiting the scan mode (e.g., to change the “horizon” line). Scrolling the touch input device 1042 may trigger a signal used to traverse a menu or highlight a portion of the menu when the menu is displayed or a menu mode is active (as illustrated in FIG. 20). Pressing the touch input device 1042 may trigger a signal used to select a highlighted portion or of the menu or feature on the menu when the menu is displayed. Scrolling the touch input device 1042 may produce a signal used to select right elbow bias when the elbow bias function is activated using the menu. Scrolling forward on touch input device 1042 may move up the menu and scrolling backwards with touch input device 1042 may move down the menu, or vice versa. Clicking first touch input device 1042 may make a selection within a menu.

FIGS. 19A and 19B depict another embodiment according to the present disclosure featuring a left hand controller 1001′ and a right hand controller 1002′. Compared with the hand controllers 1001, 1002 in FIGS. 18A and 18B, some buttons of the hand controllers 1001′, 1002′ have the same button type but different functions. For example, the second button 1035′ of the right hand controller 1002′ may trigger a signal used to turn on or turn off a menu. Compared with the hand controllers 1001, 1002 in FIGS. 18A and 18B, some buttons of the hand controllers 1001′, 1002′ may have a different button type and/or different functions. For example, touch input device 1041′ for the left hand controller 1001′ may have a three-way switch button type. Switching or holding the touch input device 1041′ to the center may trigger a signal used to engage or disengage a scan mode of the surgical robotic system. Switching the touch input device 1041′ forward may activate a zoom in function to magnify a view provided by the camera assembly of the surgical robotic system and displayed to the operator, and switching backward with first touch input device 1041′ may provide a zoom out function to reduce the view provided by the camera assembly of the surgical robotic device and displayed to the operator, or vice versa. Switching the touch input device 1035′ upward may trigger a signal used to traverse a menu when the menu is displayed or a menu mode is active. Touch input device 1042′ for the right hand controller 1002′ may have a three-way switch button type. Switching the touch input device 1042′ may trigger a signal used to traverse a menu or highlight a portion of the menu when the menu is displayed or a menu mode is active by pressing the touch input device 1035′. Switching forward on touch input device 1042′ may move up the menu and switching backwards with touch input device 1042′ may move down the menu, or vice versa. Clicking first touch input device 1042′ may trigger a signal used to select a highlighted portion or of the menu or feature on the menu when the menu is displayed. In some embodiments, switching the touch input device 1042′ may trigger a signal used to select right elbow bias when the elbow bias function is activated using the menu. Compared with the hand controllers 1001, 1002 in FIGS. 18A and 18B, the hand controllers 1001′, 1002′ may have the first paddles 1021′, 1023′ and second paddles 1022′, 1024′ to couple to finger loops 1061, 1062, 1063, 1064, respectively. Each finger loop can be a velcro type. In some embodiments (not illustrated), each finger loop can be a hook type. Deflection or depression of the first paddle 1021′, 1023′, and the second paddle 1022′, 1024′, is configured to trigger a signal to control a tool or an instrument tip (e.g., opening/closing an aperture of graspers/jaws of an instrument tip) at a distal end of a robotic arm of the surgical robotic system. For example, depressing first paddle 1021′, 1023′ and the second paddle 1022′, 1024′ may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate). Compared with the hand controllers 1001, 1002 in FIGS. 18A and 18B, first buttons 1031′, 1034′ may have a slider button type. Sliding the first button 1031′, 1034′ may trigger a signal used to control a clutch function for the corresponding hand controller of the surgical robotic system.

FIG. 20 schematically depicts a graphical user interface 1100 including a camera view portion 1110 displaying a view from the camera assembly and a menu 1120. A hand controller may select an item listed in the menu 1120, such as by controlling touch input devices.

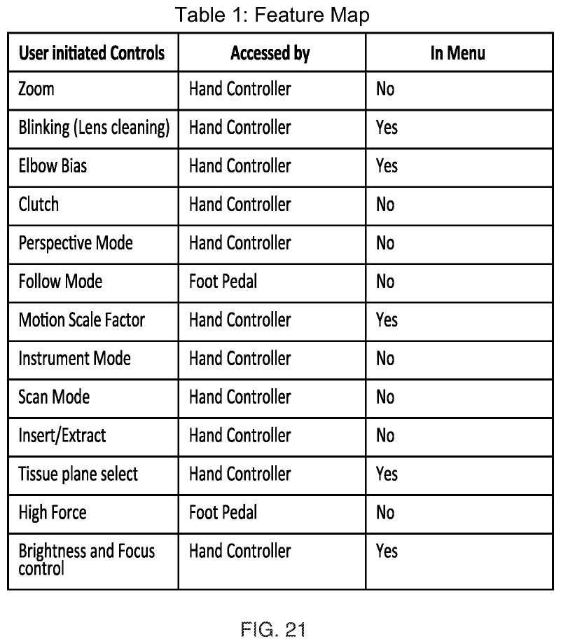

FIG. 21 is a feature map table depicting functions accessed by hand controllers, foot pedals and a menu as taught herein in some embodiments. A zoom-in/out control, a clutch control, a perspective mode, an instrument mode, a scan mode, an insertion/extraction control can be controlled by one or more hand controllers. A blinking control, an elbow bias control, a motion scale factor control, a tissue plane select control and brightness and focus control can be controlled by one or more hand controllers via the menu (as illustrated in FIG. 20). A follow mode and a high force control can be controlled by one or more foot pedals via the menu.

In some embodiments, hand controller as taught herein (e.g., as illustrated in FIGS. 6-10, 15, 16, 18, and 19) may not include engage/disengage function. Instead, an operator may put his/her head close to a display such that his/her head may enter a surgeon vigilance sensor range, the operator can squeeze paddles as taught herein (e.g., as illustrated in FIGS. 6-10, 15, 16, 18, and 19) to engage an instrument control mode. The operator may pull his/her head out of the surgeon vigilance sensor range to disengage the instrument control mode. In some embodiments, a pivot mode may be deprecated in the surgical robotic system. For example, functionalities of a pivot mode can be consolidated into a camera mode. The camera mode can have 3 degrees of freedom for controlling. The camera mode can be engaged by hitting the second button 1032 on the hand controller 1001′ and then manipulating the hand controllers 1001′, 1002′ with 3 degrees of freedom. In some embodiments, the direction of the hand controller movement can be opposite to the direction of chest movement. In some embodiments, paddles 1021-1024, 1021′-1024, and/or finger loops 1061-1064 of hand controllers 1001, 1002, 1001′, 1002′ can be automatically adjusted to be aligned with instrument tips and/or end effectors at all time. In some embodiments, a scan mode can have 2 degrees of freedom for controlling without rolling degree of freedom.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may assigned to some buttons and some touch input devices in some embodiments. One of ordinary skill of the art in view of the present disclosure will also appreciate that some embodiments may not assign some of the functions described herein or any of the functions described herein to any of the buttons and/or touch input devices of hand controllers. In some embodiments, one or more functions may be assigned to a foot pedal of a surgical robotic system that includes one or more hand controllers as described herein.

### Method for Controlling a Robotic Assembly of a Surgical Robotic System

Embodiments of the present disclosure provide a method for controlling a robotic assembly of a surgical robotic system using hand controllers (e.g., hand controllers as illustrated in FIGS. 6-10, 15, 16, 18, and 19).

FIG. 22 is a flowchart illustrating steps 2100 for controlling a robotic assembly using hand controllers carried out by a surgical robotic system in accordance with some embodiments. In step 2110, a system 10 receives a first control mode selection input. For example, while the robotic assembly 20 is disposed in an interior cavity of a subject, the system 10 can receive a first control mode selection input and change a current control mode of the surgical robotic system 10 to a first control mode in response to the first control mode selection input. The surgical robotic system can include a plurality of control modes including one or more of: a scan mode, a view mode, a travel mode, a pivot mode, and a translation mode, and the first control mode being the scan mode, the view mode, the travel mode, the pivot mode, or the translation mode. In some embodiments, the system 10 does not need to include all the control modes. For example, a pivot mode can been removed from the surgical robotic system 10. Each control mode is described below.

In step 2120, the system 10 changes positions and orientations of at least one of a camera assembly, one or more robotic arm assembly, or one or more instrument tips. For example, the system 10 can take certain actions in response to a first movement including a first translation and a first rotation of the right hand controller or the left hand controller while the current control mode is the first control mode. Where the first control mode is the scan mode, the system 10 can hold the robotic arm assembly stationary while rotating the camera assembly in a corresponding first rotation relative to a view of the camera assembly displayed on the image display of the surgical robotic system, which is a displayed camera view. Where the first control mode is the travel mode, the system 10 can move the corresponding instrument tip by a corresponding first movement including a corresponding first translation and a corresponding first rotation relative to the displayed camera view while rotating the camera assembly to center the view of the camera assembly on a position of a midpoint between instrument tips of the robotic arms of the robotic assembly, which is an average tip position, while translating the robotic assembly to translate a chest point of the virtual chest, rotating the robotic assembly to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range. Where the first control mode is the view mode, the system 10 can hold a position and an orientation of each instrument while moving the camera assembly by a corresponding first movement including a corresponding first translation and a corresponding first rotation relative to the displayed camera view and rotating a virtual chest of the robotic assembly to maintain an angular deviation between the line from the center of the virtual chest to the average instrument tip position and the normal to the virtual chest within the acceptable angular deviation range. Where the first control mode is the pivot mode, the system 10 can move a corresponding instrument tip by a corresponding first movement including a corresponding first translation and a corresponding first rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range. Where the first control mode is the translate mode, the system 10 ca move a corresponding instrument tip by a corresponding first movement including a corresponding first translation and a corresponding first rotation relative to the displayed camera view while rotating the camera assembly or rotating the camera assembly and translating the virtual chest to center the view of the camera assembly on the average tip position and to maintain a distance between the center of the virtual chest and the average instrument tip position within the acceptable distance range.

In some embodiments, the current control mode may be an instrument control mode in which a movement of the right hand controller or the left hand controller causes a corresponding movement of an instrument tip at a distal end of a corresponding robotic arm relative to the displayed camera view without moving or changing an orientation of the virtual chest of the robotic assembly. In some embodiments, the first control mode selection input may be received via a foot pedal of the surgical robotic system. In some embodiments, the first control mode selection input may be received via the first hand controller or the second hand controller. In some embodiments, the first control mode selection input may be received via a button or touch input device of the right hand controller or the left hand controller. In some embodiments, the first control mode may be the view mode. In some embodiments, the first control mode may be the scan mode. In some embodiments, the first control mode may be the travel mode. In some embodiments, the first control mode may be the transverse mode. In some embodiments, the first control mode may be the pivot mode.

In step 2130, the system 10 receives a second control mode selection input. For example, by controlling the hand controllers as described with respect to FIGS. 6-10, 15, 16, 18, and 19, an operator can select a second control mode from a plurality of control modes of the surgical robotic system. The plurality of control modes can include two or more of the scanning mode, the view mode, the travel mode, the translate mode, and the pivot mode. In some embodiments, the system 10 does not need to include all the control modes. For example, a pivot mode can been removed from the surgical robotic system 10. Each control mode is described below. The system 10 can receive a second control mode selection input via the hand controllers.

In step 2140, the system 10 can change a current control mode of the surgical robotic system to a second control mode different than the first control mode in response to the second control mode selection input.

In step 2150, the system 10 changes positions and orientations of at least one of a camera assembly, one or more robotic arm assembly, or one or more instrument tips in response to a second movement including a second translation and a second rotation of the right hand controller or the left hand controller while the current control mode is the first control mode. For example, where the second control mode is the scan mode, the system 10 can hold the robotic arm assembly stationary while rotating the camera assembly in a corresponding second rotation relative to the displayed camera view. Where the second control mode is the travel mode, the system 10 can move the corresponding instrument tip by a corresponding second movement including a corresponding second translation and a corresponding second rotation relative to the displayed camera view while rotating the camera assembly to center the view of the camera assembly on the an average tip position, while translating the robotic assembly to translate the chest point of the virtual chest, rotating the robotic assembly to rotate the virtual chest, or both, to maintain the distance between the center of the virtual chest and the average tip position in the acceptable distance range, and to maintain an angular deviation between the line from the chest point to the average tip position and the normal to the virtual chest within the acceptable angular deviation range. Where the second control mode is the view mode, the system 10 can hold a position and an orientation of each instrument while moving the camera assembly by a corresponding second movement including a corresponding second translation and a corresponding second rotation relative to the displayed camera view and rotating the virtual chest of the robotic assembly to maintain the angular deviation between the line from the center of the virtual chest to the average instrument tip position and the normal to the virtual chest within the acceptable angular deviation range. Where the second control mode is the pivot mode, the system 10 can move a corresponding instrument tip by a corresponding second movement including a corresponding second rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range. Where the second control mode is the translate mode, the system 10 can move a corresponding instrument tip by a corresponding second movement including a corresponding second translation and a corresponding second rotation relative to the displayed camera view while rotating the camera assembly or rotating the camera assembly and translating the virtual chest to center the view of the camera assembly on the average tip position and to maintain the distance between the center of the virtual chest and the average instrument tip position within the acceptable distance range. In some embodiments, the system 10 does not need to include all the control modes. For example, a pivot mode can been removed from the surgical robotic system 10.

### Control Modes and Features for Control of a Surgical Robotic System

Certain control modes and features for control of a surgical robotic system are described below in greater detail. Some embodiments may include all of the modes and features described herein. Some embodiments may include only some of the modes and features described herein. It may be appreciated that embodiments may optionally be provided with certain modes based upon the procedures intended to be performed or suitable for the surgical robotic system in order to provide improved functionality for a particular purpose, however, it should also be appreciated that some embodiments according to the present technology may not be provided with any particular mode or features or set of modes or features.

Some embodiments described herein provide systems and methods employing a plurality of different control modes for controlling a robotic assembly of a surgical robotic system, when the robotic assembly is disposed within an internal body cavity of a subject.

Each control mode uses sensed movement of one or more hand controllers, and may also use input from one or more foot pedals, to control the robotic arm assembly and/or the camera assembly. A control mode may be changed from a current control mode to a different selected control mode based on operator input (e.g., provided via one or more hand controllers and/or a pedal of the surgical robotic system). In different control modes, the same movements of the hand controllers may result in different motions of the robotic assembly.

Some embodiments employ a plurality of control modes including an instrument control mode, which may also be referred to as an “instrument mode” herein, as well as one or more of a view control mode, which may also be referred to as a “view mode”, a “camera control mode”, a “camera mode”, a “framing control mode”, a “framing mode”, a “perspective mode” herein, a scan mode, which may also be referred to herein as a “scanning mode” or a “survey mode”, and a travel control mode, which may also be referred to as a “travel mode” or an “autotrack mode” herein. The pivot mode, the travel mode and the translate mode may all be referred to as “tracking modes” herein.

Some embodiments employ a graphical user interface that identifies a current control mode of the surgical robotic system. Some embodiments employ a menu feature in which a menu is displayed on the graphical user interface and inputs from one or more of the hand controllers can be used to traverse menu options and select menu options.

Some embodiments employ additional features for controlling the robotic assembly. For example, some embodiments enable individual control of an elbow bias or an elbow elevation of a right robotic arm and a left robotic arm.

### Graphical User Interface for Operator Display

In some embodiments, a graphical user interface (GUI) of the surgical robotic system includes a display for an operator including a camera view portion and at least one informational portion that identifies a current control mode of the surgical robotic system.

An example GUI is described with respect to FIG. 17. In some embodiments, the identification of a current control mode of the surgical robotic system in the at least one informational portion may include words, colors, patterns, symbols or any combination of the aforementioned.

### Instrument Control Mode

As described above, some embodiments employ or provide an instrument control mode, which may be described as an “instrument mode” herein. In the instrument mode, the surgical robotic system identifies movement (e.g., translation and/or rotation) of each hand controller and moves (e.g., translates and/or rotates) an instrument tip on an distal end of the corresponding robotic arm in a corresponding matter. In the instrument control mode, the surgical robotic system may cause an instrument tip to move in a manner directly proportional to movement of a corresponding hand controller. This may be described as motion including translation and/or rotation of the instrument tip of a robotic arm being directly controlled by motion of respective hand controller. For example, translating a hand controller in a direction by an amount causes the corresponding instrument tip for the corresponding robotic arm to move in a corresponding direction (i.e., in the same direction with respect to a view from the camera assembly displayed to the operator) by a corresponding scaled down amount (e.g., where the scaling is based on the scale of the view from the camera assembly displayed to the operator). As another example, rotating a hand controller about an axis by an angle causes the corresponding instrument tip for the corresponding robotic arm to rotate by a same angle about to a corresponding axis (e.g., where the corresponding axis is a same axis with respect to the orientation of the view from the camera assembly displayed to the operator). In the instrument control mode, operator controls can be used to actuate instruments (e.g., via grasper controls of a hand controller, via foot pedal controls) as well as we as to move or change an orientation of instrument tips.

In the instrument mode, movement of the hand controllers does not change a position and does not change an orientation of the camera assembly (e.g., the camera assembly orientation and position may remain fixed) and does not change a position or an orientation of the virtual chest. In other words, the instrument mode does not reposition or reorient the camera or the chest. The instrument control mode is useful for manipulating the instrument tips within a working area of an internal body cavity that is accessible without moving a virtual chest of the robotic assembly.

In some embodiments, the operator can enable or disable the instrument control mode via either or both of the hand controllers. In some embodiments, an instrument mode is engaged and disengaged using an input control from a hand controller (e.g., by pressing a button, such as button 233 of in FIG. 6A or button 333 in FIG. 7A, or interacting with a touch input device, such as touch input device 443 in FIG. 9A). When the instrument mode is disengaged, any movement of a hand controller does not cause any corresponding movement of the associated instrument tip. In some embodiments, when the surgical robotic system is in a disengaged state, an information portion of the GUI of the user display indicates that the current state is disengaged. In some embodiments, engaging the clutch causes an information panel of the graphical user interface to identify that the clutch is engaged (e.g., via text, color, or any other graphical indicator)

In some embodiments, the instrument control mode is a default control mode that the surgical robotic system enters when another control mode, such as the view control mode, or the travel control mode is exited.

### Further Description of Control Modes

As described herein, some embodiments provide one or more of a scan mode, a camera/view mode, a travel mode, a pivot mode, or a travel mode in addition to an instrument control mode Further description of these modes is provided below.

### Scan Mode

As described above, some embodiments employ or provide a scan mode, which may also be described as a “scan control mode”, or a “scanning mode” herein. In the scan mode, rotation of one of the hand controllers causes a corresponding rotation of the camera assembly (e.g., a yaw rotation, a pitch rotation, a roll rotation, or a combination of the aforementioned) to change a view provided by the camera assembly. In the scan mode, movement of either of the hand controllers causes no movement of the robotic arms and causes no movement of the virtual chest of the robotic assembly. Scan mode may be used to quickly survey an interior body cavity, to check an elbow position of robotic arms, and or to look for surgical materials.

In some embodiments, the scan mode is entered and excited, which may be described as the scan mode being activated and deactivated, using an input from one or both of the hand controllers or using an input from one or both of the hand controllers in combination with an input from a foot pedal (e.g., via input touch device 241 in FIG. 6A, via input touch device 341 in FIG. 7A). In some embodiments, when the scan mode is exited, an orientation and position of the camera assembly returns to the orientation and position that the camera assembly had when the scan mode was entered.

In some embodiments, when the surgical system is in the scan mode, in response to a first movement including a first rotation of the right hand controller or the left hand controller, the robotic arm assembly is held stationary while the camera assembly is rotated in a corresponding first rotation relative to a view of the camera assembly displayed on the image display, which is a displayed camera view

### View Control Mode/Camera Control Mode

As described above, some embodiments include a view control mode, which may also be referred to as a “view mode”, a “camera control mode”, a “camera mode”, a “framing control mode”, a “framing mode”, or “perspective mode” herein. In view control mode or camera mode, movement (e.g., translation and/or rotation of one of the hand controllers causes a corresponding movement (e.g., translation and/or rotation) of the camera assembly. A movement of the camera assembly can include, but is not limited to a forward/backward translation, a vertical translation, a lateral translation, a yaw, a pitch, a roll, or any combination of the aforementioned. In the view control mode/camera mode, instrument tips of the robotic arms remain stationary, but other portions of the robotic arms may move to accomplish the corresponding motion of the camera assembly. For example, the virtual chest of the robotic assembly may need to translate and/or change its orientation to achieve the movement of the camera assembly. By keeping the instrument tips in a fixed position and orientation, the view control mode enables an operator to frame a particular view, such as a view of a portion of the internal cavity in which a procedure is being performed, while not moving the instrument tip or any tissue in contact therewith.

For example, in some embodiments, when the surgical robotic system is in the camera control/view mode, in response to a first movement including a first translation or a first rotation of the right hand controller or the left hand controller or both simultaneously, a position and an orientation of each instrument is held while the camera assembly is moved by a corresponding first movement including a corresponding first translation and a corresponding first rotation relative to the displayed camera view and a virtual chest of the robotic assembly is rotated to maintain an angular deviation between the line from the center of the virtual chest to the average instrument tip position and the normal to the virtual chest within the acceptable angular deviation range.

In some embodiments, a camera control mode is entered and exited in response to an operator input (e.g., an operator input via a foot pedal, which may be a dedicated camera control foot pedal or a button on one of the hand controllers). In some embodiments, when the camera control mode is exited, a framing or view is maintained, meaning that a position and an orientation of the camera control assembly is maintained

In some embodiments, a camera control mode can include functionalities of a pivot mode as described with respect to a section of a pivot mode. For example, in addition to functionalities described above, a camera control further can enable an operator to control the positions and orientations of the instrument tips by corresponding movements of the hand controllers, while changing an orientation of the camera or an orientation of the camera assembly and an orientation of the virtual chest to maintain a center of view of the camera assembly on the average instrument tip position. For example, when in the camera control mode, in response to a first movement including a first translation and a first rotation of the right hand controller or the left hand controller, a corresponding instrument tip is moved by a corresponding first movement including a corresponding first rotation relative to the displayed camera view, the camera assembly is rotated to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range.

### Travel Mode

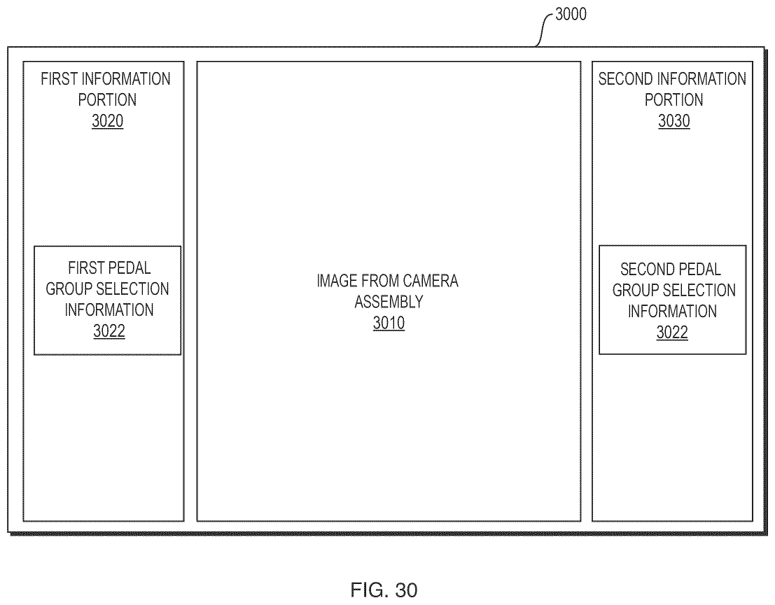

As described above, some embodiments include a travel control mode, which may also be referred to as a “travel mode” herein. When the travel control mode is activated, movements of the left hand controller and the right hand controller are translated into corresponding movements of end effectors or instrument tips of the robotic assembly. Similar to the instrument control mode, instrument and tools can be actuated in the travel control mode. Unlike the instrument control mode, in the travel control mode, the camera assembly and the chest track a midpoint between an instrument tip or tips of the right robotic arm and an instrument tip or tips of the left robotic arm. Unlike the instrument control mode, in the travel mode, movement of the hand controllers can also cause displacement and/or a change in orientation of a chest of the robotic assembly, enabling the robotic assembly to “travel” or “follow” the instruments tips. This may be described as the instrument tips directing or leading movement through an interior body cavity. For example in some embodiments the surgical robotic system establishes a cone of movement (e.g., an acceptable range for a distance of the instrument tips from the virtual chest of the robotic assembly position, and an acceptable range of deviation for a line connecting the center of the chest to the instrument tips from a normal of the chest), and when the instrument tips or end effectors would exceed the cone of movement, the chest and arms of the robotic system automatically move to keep the instrument tips within the cone of movement (see FIG. 30). The cone of movement may not have a constant length or a constant angular range, but the length and the angular range may vary during use based on some other parameters of the surgical robotic system, or based on currently selected features and options of the surgical robotic system (e.g., based on a zoom of the camera view displayed).

For example, in some embodiments, when in the travel mode, in response to a first movement including a first translation and a first rotation of the right hand controller or the left hand controller or both, a corresponding instrument tip is moved by a corresponding first movement including a corresponding first translation and a corresponding first rotation relative to the displayed camera view while the camera assembly is rotated to center the view of the camera assembly on a position of a midpoint between instrument tips of the robotic arms of the robotic assembly, which is an average tip position, while robotic assembly is translated to translate a chest point of the virtual chest, the robotic assembly is rotated to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range. In some embodiments, there is a threshold that defines an allowable translational or rotational offset that the instrument tips may have from the virtual center/chest with which no virtual center/chest movement occurs. Once that threshold is exceeded the system would translate or rotate the virtual chest to recenter the instrument tips appropriately.

In some embodiments, the travel control mode may be used to navigate the robotic assembly to another location in an internal body cavity of a patient or to maintain visualization while a surgical task is performed. As noted above, in travel mode, the camera assembly automatically tracks the midpoint between the instrument tips during movement of the instrument tips. Accordingly, travel mode may be useful for navigation and visualization for a task because the user will be able to maintain a consistent view of the instrument tip. This visualization may be valuable, for example, in procedures such as suturing the circumference of a mesh or creating a flap.

### Pivot Mode

As described above, some embodiments include a pivot control mode, which may also be referred to as a “pivot mode” herein. The pivot mode enables an operator to control the positions and orientations of the instrument tips by corresponding movements of the hand controllers, while changing an orientation of the camera or an orientation of the camera assembly and an orientation of the virtual chest to maintain a center of view of the camera assembly on the average instrument tip position. For example, when in the pivot mode, in response to a first movement including a first translation and a first rotation of the right hand controller or the left hand controller, a corresponding instrument tip is moved by a corresponding first movement including a corresponding first rotation relative to the displayed camera view, the camera assembly is rotated to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range.

In some embodiments, engagement of a foot pedal activates the pivot mode. In some embodiments, engagement of an input function on a hand controller activates the pivot mode.

In some embodiments, the functionality of the pivot mode may be consolidated into the camera mode and the system does not need to have a pivot mode such that a camera mode can perform functionalities described with respect to the section of “View Control Mode/Camera Control Mode” and functionalities of the pivot mode as described herein.

### Translate Mode

As described above, some embodiments include a translate control mode, which may also be referred to as a “translate mode”, a “translation mode”, or a “translation control mode” herein. As described above, some embodiments include a travel control mode. The translation control mode enables an operator to control the positions and orientations of the instrument tips by corresponding movements of the hand controllers, while changing an orientation of the camera assembly and/or translating the chest of the robotic assembly to maintain a center of view of the camera assembly on the average instrument tip position and to keep an average distance between the center of the chest and the average tip position in an acceptable range. For example, when in the translate mode, in response to a first movement including a first translation and a first rotation of the right hand controller or the left hand controller, the corresponding instrument tip is moved by a corresponding first movement including a corresponding first translation and a corresponding first rotation relative to the displayed camera view while camera assembly is rotated or the camera assembly is rotated and the virtual chest is translated to center the view of the camera assembly on the average tip position and to maintain a distance between the center of the virtual chest and the average instrument tip position within an acceptable distance range.

In some embodiments, engagement of a foot pedal activates the travel mode. In some embodiments, engagement of an input function on a hand controller activates the travel mode

Methods for Implementing Instrument Mode, Travel Mode, and View Mode with Respect to the Camera Center, the Camera Direction and the Position and Orientation of the Virtual Chest

Some embodiments implement methods and modes for controlling movements of at least portions of a robotic assembly (e.g., robotic arms) to achieve desired instrument tip locations and orientations. In some embodiments, methods for control abstracts away from direct and separate control of a virtual chest of a robotic assembly and a camera assembly robot's chest enabling an operator to focus entirely on the robotic assembly's instrument tips and the operator's view of those instrument tips. Definitions of terms to be used to explain control methods are included below.

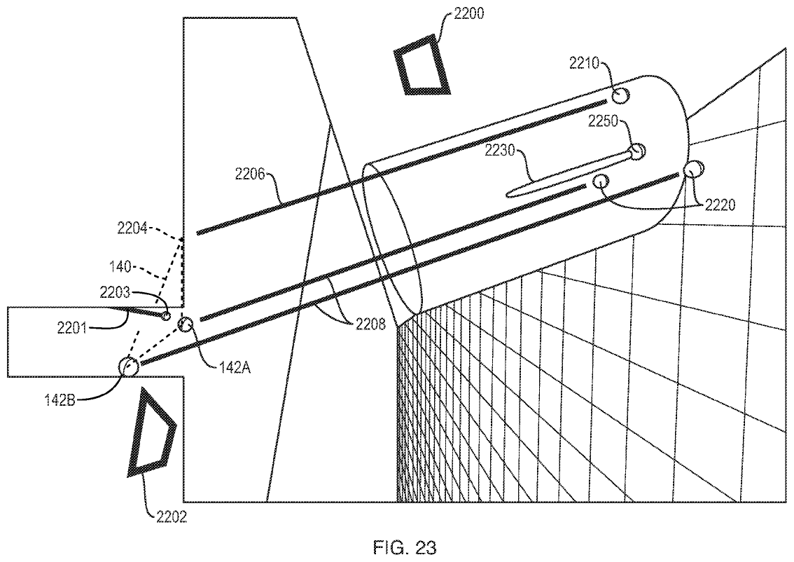

As used herein a “chest point” refers to a center point of a virtual chest of a robotic assembly. As used herein a “chest normal” refers a direction of the virtual chest, which is also a normal to the chest plane. As used herein a “chest plane” is a plane that passes through the chest point whose normal is the chest direction. As used herein, a “trocar center” is a point in space around which a trocar inserted in the patient and supports that extend through the trocar for supporting the robotic assembly within the internal body cavity pivot. As used herein, a “trocar direction” is a direction from the trocar center to the chest point. As used herein, the “trocar plane” is a plant that passes through the trocar center whose normal is the trocar direction. As used herein, the “average tip position” is a point in space that is half-way between the instrument tips of the robotic arms. As used herein, the “camera origin” is a neutral position of the camera assembly relative to a support of the camera assembly. In some embodiments, a camera assembly may be able to plunge forward and withdraw back relative to the support of the camera assembly. As used herein, a “shoulder” of an arm is the beginning of the arm or the location of the most proximal joint of the arm. As used herein, a “camera/arm root” is a point at which the camera or arm support intersects the trocar plane. As used herein, the “root triangle” is a triangle formed by the camera root and the two arm roots. It is always coplanar with the trocar plane. FIG. 23 schematically depicts the relationship between these centers, planes, and directions.

One skilled in the art will appreciate that the term “paddle,” “paddles” may also be referred to as control lever, control levers.

In some embodiments, some control modes and methods, such as the travel mode, focus on maintaining an “ideal robot pose”, which may also be referred to as a “desired robot pose”, specifically pertaining to the positions/orientations of the shoulders of the robotic arms, the camera assembly, and the trocar.

### Controlling the Position and Orientation of the Instrument Tips in the Instrument Control Mode

The operator controls the position and orientation of the instrument tips using two hand controllers, one mapped to each instrument tip. Using an operator input (e.g., on a hand controller or via a foot pedal) the user can enable and disable control of each instrument tip, which may be described as engaging and disengaging from an instrument control mode. When the instrument control mode is enabled, moving and rotating the hand controller moves and rotates the instrument tip in a similar manner. When the instrument control mode is disabled or disengaged, moving a hand controller does not cause any movement of the corresponding instrument tip. The instrument control mode can be employed to achieve a “ratcheting” type of motion for larger movements by turning the instrument control off (e.g., disengaging the instrument control mode or engaging the clutch), repositioning one or both of the hand controllers, re-enabling instrument control (e.g., engaging the instrument control mode or disengaging the clutch), and continuing the movement.

The translational movements of the instrument tips are relative to the current orientation of the camera. For example, engaging the instrument control mode and moving a hand controller forward causes the corresponding instrument tip to move forward in the frame of reference of the camera view (i.e. the direction that the camera is currently pointed). Moving the hand controller up causes the instrument tip to move in the up direction of the camera's frame of reference (i.e. the hand moves up with respect to the frame of view of the camera image displayed to the operator, which is the “base” of the camera frustum). In some embodiments, the scale of these movements is different in different modes.

Rotational movements of the hand controller map to rotations relative to the current orientation of the instrument tip. For example, rolling the hand controller left causes the instrument tip to rotate a similar amount around its forward axis, pitching the hand controller up causes the instrument tip to pitch a similar amount around its leftward axis, and so on.

### The “Chest” and “Camera Offset”

FIG. 23 schematically depicts a trocar plane 2200, a chest plane 2202 and various points, and directions associated with a virtual chest 140 and camera assembly 44 of a robotic assembly 20 and with a trocar 50 in accordance with some embodiments.

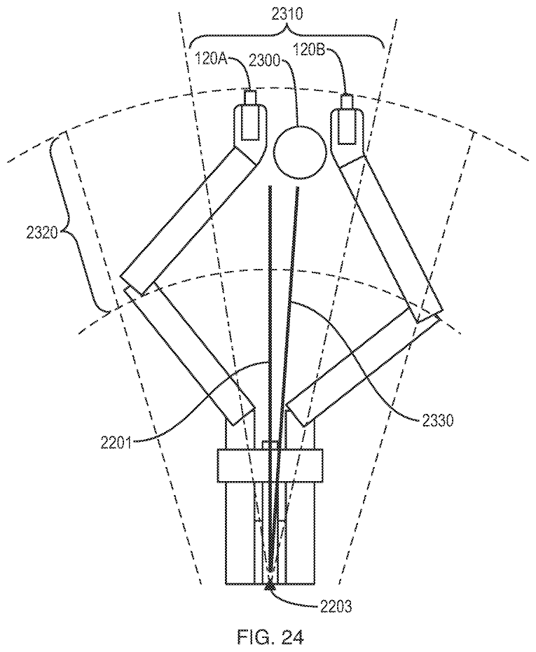

A chest point can be a center point 2203 of a virtual chest 140 of a robotic assembly 20. A chest normal 2201 can be a direction of the virtual chest 140, which is also a normal to the chest plane 2202. A chest plane 2202 can be a plane that passes through the chest point 2203 whose normal is the chest direction 2201. A trocar center 2250 can be a point in space around which a trocar 50 inserted in a patient and supports that extend through the trocar 50 for supporting the robotic assembly 20 within the internal body cavity pivot. A trocar direction 2230 can be a direction from the trocar center 2250 to the chest point 2203. A trocar plane 2200 is a plane that passes through the trocar center 2250 whose normal is the trocar direction. An average tip position 2300 (as illustrated in FIG. 24) can be a point in space that is half-way between the instrument tips 120A, 120B of the robotic arm assemblies 42A, 42B (as illustrated in FIG. 5). A camera origin 2204 can be a neutral position of the camera assembly 44 relative to a support of the camera assembly 44. In some embodiments, a camera assembly 44 may be able to plunge forward and withdraw back relative to the support of the camera assembly 44. A shoulder 126 of a robotic arm assembly 42 can be a beginning of the robotic arm assembly 42 or a location of the most proximal joint of the robotic arm assembly 42 (as illustrated in FIG. 5). A camera root 2210 can be a point at which the camera assembly support intersects the trocar plane 2200. An arm root 2220 can be a point at which the robotic arm assembly 44 support intersects the trocar plane. A root triangle can be a triangle formed by the camera root 2210 and the two arm roots 2220. It is always coplanar with the trocar plane 2200.

Physically, the virtual chest 140 can be a triangle formed by the two shoulders 126A, 126B and a camera origin 2210. Conceptually, it is a projection of a root triangle onto a chest plane 2202. To calculate this projection, rays 2206, 2208 are cast from a camera root 2210 of the camera assembly 44 and arm roots 2220 of the robotic arm assemblies 42A, 42B in a trocar direction 2230 and intersected with the chest plane 2202. Each drive moving a portion of the robotic assembly 20 should insert and outsert (e.g., withdraw) to keep the shoulders 126A, 126B and camera origin 2204 on the chest plane 2202.

During operation, the camera assembly 44 always orients itself relative to the chest plane 2202. That is to say, as the chest plane 2202 rotates, the camera assembly 44 can rotate in a similar manner. The operator can introduce an offset (called the Camera Offset) to change the angle of the camera assembly 44 relative to the chest plane 2202.

### Controlling the Robotic Body in the Travel Mode

Aspects of a method for implementing the travel mode are described below in accordance with some embodiments. In other embodiments there may be different or other implementations of a travel mode. In the travel mode, positioning of the robotic body (e.g., the virtual chest) is primarily determined by the position of the instrument tips. In some embodiments, as the user is working and controlling the instrument tips, the chest is repositioned to maintain a desired arm configuration, which may be an “ideal” arm configuration. In some embodiments, as the user is working and controlling the instrument tips, the chest is repositioned to maintain a configuration that the robotic assembly was in upon entering the travel mode and upon exiting the travel cone, which may be the desired arm configuration. This requires moving (i.e. translating and/or rotating) the virtual chest to reposition the chest point and reorient the chest as needed to maintain the desired arm configuration. In some embodiments, yaw and pitch of the positioning arm may also be employed to achieve a desired positioning of the robotic assembly.

The method for repositioning the Chest Point tries to keep the Average Tip Position within a certain distance range at all times. It is as follows:
1) “Draw” a line from the Chest Point to the Average Tip Position, and measure its length.
2) If the length of that line is over some value (e.g. 12 cm}, translate the Chest Point toward the Average Tip Position.
3) If the length of that line is under some value (e.g. 6 cm), translate the Chest Point away from the Average Tip Position.
4) If the length of that line is between those two values, do not translate the Chest Point.

In some embodiments, a “soft boundary” could be implemented where the chest moves slowly when the distance is in a certain range, e.g., between 10 cm and 12 cm, and quickly when the distance is greater than that range, e.g., greater than 12 cm, or by having the speed with which the chest moves be proportional to the distance.

The method for reorienting the chest seeks to keep the chest facing or nearly-facing the instrument tips. It is as follows:
1) “Draw” a line from the Chest Point to the Average Tip Position.
2) If the angle between that line and the Chest Normal is over a given value (e.g. 10 degrees), rotate the Chest Normal towards that line.
3) If the angle is less than the given value, do not rotate the Chest Normal.

In some embodiments this method of reorienting the chest may also include a soft boundary condition on when the Chest Normal rotates.

FIG. 24 schematically depicts positioning and orienting the chest plane 2202 to keep a distance 2330 from the chest point 2203 to the average instrument tip position 2300 in acceptable range 2320 and to keep an angular deviation of the average instrument tip position 2300 from the chest normal/chest direction 2201 in an acceptable range 2310 in accordance with some embodiments. The net result of the combined chest reorienting and chest translating is that the robot chest stays properly positioned and oriented towards the operator's working space. As mentioned above, while the chest point 2203 and chest normals 2201 change, the trocar 50 should pivot and the robot drives should insert/outsert in order to keep the trocar direction 2230 correct and the shoulder 126/camera origin 2204 on the chest plane 2202.

As the chest moves/rotates, the chest normal 2201 may become perpendicular or nearly-perpendicular to the trocar direction 2230. This will result in a very distorted projection for the chest, and would require the shoulders 126 or camera origin 2204 to be very far apart from one another. To prevent this, the chest normal 2201 can be restricted to always being more than a specified minimum angular amount (e.g., more than 20 degrees) away from the perpendicular to the trocar direction 2230.

### Controlling the View in the View Control Mode

At any point, the operator can enable View Control mode (e.g., using a toggle control such as a foot pedal or a toggle control). Once in View Control mode, controls for both robot instrument tips are disabled and can't be re-enabled until the user exits View Control mode.

While in View Control mode, the user can rotate a single hand controller to rotate the robot camera. The rotation of the hand controller maps directly to the rotation of the robot camera. Thus, rotating the hand controller up around its left axis causes the camera to rotate a by a corresponding scaled amount (e.g., scaled down) around its left axis, and yawing the controller to the right causes the camera to rotate a similar amount around its up axis.

Moving the hand controller has varying effects based on the direction of motion. Movements along the hand controller's forward axis cause the robot camera to plunge forward or backward. Note that this does not change the position of the Camera Origin, since the Chest Normal does not change. This merely changes the robot camera's distance from the Camera Origin.

Movements along the hand controller's left-right and vertical axes cause the Chest Point to move. All of these movements are relative to the robot camera's frame of reference. Thus, moving the hand controller up causes the Chest Point to move in the direction of the robot camera's up axis, and moving the hand controller left causes the Chest Point to move in the direction of the robot camera's left axis.

In some embodiments, none of the different controls in View Control mode are exclusive; they are all active at the same time, allowing for smooth, combined plunging, rotating, and chest positioning.

In some embodiments, when the operator exits the View Control mode, the angular offset between the camera's orientation and the orientation of the chest is stored as the Camera Offset. At any time, the user can press a “reset” button which causes the Camera Offset to slowly return to 0.

### Advantages Over Control Methods in Some Conventional Surgical Robotic Systems

Some conventional surgical robotic system employ gestural control in a dedicated travel mode or travel state to travel move a robotic assembly within an internal body cavity. For example in one conventional system, an operator grabs and “pulls” the target workspace toward the operator while in the dedicated travel control mode. The travel mode described herein offers several advantages over this “grab and pull” method. First, in the travel mode described herein, an operator can both operate the instruments and travel when needed within a single control mode. In contrast, the “grab and pull” method requires the operator activate a discrete dedicated travel mode, move robotic assembly, and then return to a separate control mode for robotic arm manipulation. This increases the cognitive load on the operator and requires more time and more steps. In contrast, in the travel mode described herein, the workspace moves with the instrument tips as needed as the operator is normally working, effectively removing the need for a separate dedicated “travel” control mode in many. Second, gesture control is not discoverable (e.g., gestures needs to be explicitly taught to and remembered by the operator), in contrast to movement in the present travel control mode, which can be discovered through normal operation of the surgical robotic system. Finally, travel control mode does not require the operator to understand and keep track positions of the individual components of the robot assembly (e.g., the chest, the camera, etc.), but instead requires the operator to focus on controlling the two things that the operator would be most concerned with during any normal endoscopic surgery: the instrument tips and the surgical camera view. This is substantially simpler than controlling individual components, which potentially reduces the barrier to entry for use of the surgical robotic system.

### Rotational Scaling for Suturing Motion

Some embodiments of hand controllers according to the present disclosure may be used to control suturing by the surgical robot. Moving the controller in a suturing motion sufficient for the robotic arm to perform a suture may require a greater roll motion than the human wrist is able to perform. Accordingly, in embodiments rotational scaling may for provided for a suturing motion when a suturing mode is selected.

Some embodiments may provide a suturing mode, in which some rotational movements of a hand controller result in rotations of a larger angle at an instrument tip. For example, in some embodiments of a suturing mode, the surgical robotic system performs a mathematical operation to yield a greater roll output to the surgical robotic arm than the roll movement made with the hand controller. In some embodiments, the mathematical operation may be as follows:

$${α}_{commanded} = S \times α$$

$${β}_{commanded} = β$$

$${γ}_{commanded} = γ$$

where α is the roll input from the hand controller; β is the pitch input from the hand controller; γ is the yaw input from the hand controller; S is a scale factor; α_{commanded} is the roll output sent to the robotic surgical device; β_{commanded} is the pitch output sent to the robotic surgical device; and γ_{commanded} is the yaw output sent to the surgical device.

The scale factor (S) may be chosen to be greater than 1. For example, the scale factor may be 1.1, 1.2, 1.3, 1.4, 1.5, 1.6, 1.7, 1.8, 1.9, or 2.0. The scale factor 1.4 has been tested and found to be satisfactory. When the scale factor is applied, the surgical robotic device exhibits a greater roll of the virtual wrist than has been performed by the wrist of the user and reflected by the hand controller.

### Foot Pedal Array for a Surgical Robotic System

Embodiments of the present disclosure provide a foot pedal array for receiving input from an operator to control a robotic assembly of a surgical robotic system, an operator console including such a foot pedal array, and surgical robotic systems including such a foot pedal array. Some embodiments include pairs of foot pedals and column sensors that enable detection of an operator's foot being disposed over a column of foot pedals or over a foot pedal. In some embodiments, column sensors and at least one row sensor are employed to detect an operator's foot being disposed over a specific foot pedal. In some embodiments, column sensors or column sensors and at least one row sensor may be used to identify that an operator's foot is positioned over a foot pedal in a location that obstructs a beam of the sensor(s) prior to an operator pressing or engaging the foot pedal. In some embodiments, the surgical robotic system may provide information to the user identifying the foot pedal, or the function of the foot pedal, proximate to the operator's foot in response to the sensor data. This information may permit the operator to confirm the foot pedal to be pressed or engaged before the user presses the foot pedal, which reduces the risk of inadvertent function actuation based on engaging a pedal. This may reduce risk associated with the user selecting an incorrect foot pedal, for example, as a result of being unable to see a foot pedal positioned on the foot pedal array under the user console. In embodiments in which some foot pedals are used to control an electrosurgery device or system, such a safety function could be particularly advantageous.

Some systems provide for control of modes or functions of the surgical robotic system using the foot pedal array. In some embodiments, one or more foot pedals or pairs of foot pedals may select one or more modes of the surgical robotic device; one or more foot pedals or pairs of foot pedals may operate a function or functions the surgical robotic system; one or more foot pedals or pairs of foot pedals may control articulation and motion of the surgical robot; one or more foot pedals or pairs of foot pedals may control visualization via the surgical robotic system. For example, in some embodiments, one or more pairs of foot pedals may control an electrosurgery function of the surgical robotic system and a pair of foot pedals may be used to select control modes of the surgical robotic system, including, for example, a travel mode and a camera mode, which may also be referred to herein as a view mode, in which input via a at least one hand controller of the operating system (e.g., sensed movement of the at least one hand controller) causes a different movement of one or more robotic arms of the surgical robotic system and/or of a camera assembly of the surgical robotic system than if the robotic system was in a standard mode (e.g., a mode in which the one or more robotic arms would make movements corresponding to the sensed movement of the at least one hand controller).

Notably, the present disclosure is not limited to a travel mode and a camera mode and may also include other articulation and visualization functions of the system. For example, in some embodiments the control modes may include a pivot mode and a translation mode. In some embodiments, such foot pedal control of changing modes of the surgical robotic system may decrease complexity of one or more hand controllers and may reduce operator fatigue relative to control of changing modes of operation via the hand controllers.

Further advantages of some embodiments of the present technology may include an increase in the speed and efficiency in operator control of the system as a result of easier access to modes and controls and increased confidence due to the safety advantages of the sensor system in embodiments having a sensor system. This may reduce the overall time of a surgical procedure providing patient health benefits.

Embodiments include foot pedal assemblies for use with a robotic surgical system, operator or surgeon consoles including such foot pedal assemblies, and robotic surgical systems including such foot pedal assemblies.

In one aspect, the present disclosure is directed to a foot pedal assembly for a surgical robotic system including a foot pedal array for receiving input from an operator to control a robotic assembly of a surgical robotic system. The foot pedal array includes a foot pedal tray having a left foot portion corresponding to a portion of the foot pedal tray more readily accessible by the operator's left foot and a right foot portion corresponding to a portion of the foot pedal tray more accessible to the operator's right foot when the operator is seated or standing facing the foot pedal array. The foot pedal tray also includes a first foot pedal tier, and a second foot pedal tier disposed lower than the first foot pedal tier and forward of the first foot pedal tier and extending toward the operator in use. The foot pedal assembly also includes a first foot pedal pair located on the first foot pedal tier in the right foot portion, the first foot pedal pair comprising a first foot pedal and a second foot pedal adjacent to the first foot pedal, and a second foot pedal pair located on the second foot pedal tier and in the right foot portion, the second foot pedal pair comprising a third foot pedal and a fourth foot pedal adjacent to the third foot pedal. In some embodiments, the foot pedal assembly also includes a third foot pedal pair located in the left foot portion, the third foot pedal pair comprising a fifth foot pedal and a sixth foot pedal. The foot pedal assembly further includes a first column sensor and a second column sensor. The first column sensor includes a first emitter configured to direct a first beam over at least a portion of the first foot pedal and the third foot pedal, and a first receiver configured to receive the first beam. The second column sensor includes a second emitter configured to direct a second beam over at least a portion of the second foot pedal and the fourth foot pedal, and a second receiver configured to receive the second beam.

In some embodiments, the second foot pedal tier has a height, as compared to a floor, that is less than a height, as compared to the floor, of the first foot pedal tier when the foot pedal array is in use.

In some embodiments, the first receiver and the second receiver are disposed on or inset into the second foot pedal tier of the foot pedal tray.

In some embodiments, the first column sensor is configured to produce a signal indicating a partial or full interruption in the first beam received at the first receiver caused by an object disposed between the first emitter and the first receiver, to produce a signal indicating an uninterrupted first beam received at the first receiver, or both. In some embodiments, the second column sensor is configured to produce a signal indicating a partial or full interruption in the second beam received at the second receiver caused by an object disposed between the second emitter and the second receiver, to produce a signal indicating an uninterrupted second beam received at the second receiver, or both.

In some embodiments the foot pedal array is configured such that a foot positioned over and proximate to or on the first foot pedal or the third foot pedal produces a signal indicating a partial or full interruption in the first beam; and a foot positioned over and proximate to or on the second foot pedal or the fourth foot pedal produces a signal indicating a partial or full interruption in the second beam.

In some embodiments, the first emitter and the second emitter are disposed on the second foot pedal tier beyond the first foot pedal and the second foot pedal.

In some embodiments, the fifth foot pedal is located on the first foot pedal tier and the sixth foot pedal is located on the second foot pedal tier. In some embodiments in which the fifth foot pedal is located on the first foot pedal tier and the sixth foot pedal is located on the second foot pedal tier, the foot pedal array further includes a third column sensor including: a third emitter configured to direct a third beam over at least a portion of the fifth foot pedal and the sixth foot pedal; and a third receiver configured to receive the third beam.

In some embodiments, the fifth foot pedal and the sixth foot pedal are both located on the second foot pedal tier or both located on the first foot pedal tier. In some embodiments in which the fifth foot pedal and the sixth foot pedal are both located on the same foot pedal tier, the foot pedal array further includes a third column sensor and the fourth column sensor. The third column sensor includes a third emitter configured to direct a third beam over at least a portion of the fifth foot pedal and a third receiver configured to receive the third beam. In some embodiments, the foot pedal array also includes a fourth column sensor including: a fourth emitter configured to direct a fourth beam over at least a portion of the sixth foot pedal; and a fourth receiver configured to receive the fourth beam.

In some embodiments, the foot pedal array further includes a first row sensor including: a first row emitter configured to direct a first row beam along the first foot pedal tier and over at least a portion of the first foot pedal and the second foot pedal, or along the second foot pedal tier and over at least a portion of the third foot pedal and the fourth foot pedal; and a first row receiver configured to receive the first row beam. In some embodiments, the first row beam is also directed over at least a portion of the fifth foot pedal, over at least a portion of the sixth foot pedal, or both. In some embodiments, the first row beam is directed along a different foot pedal tier than that of the fifth foot pedal and the sixth foot pedal.

In some embodiments, first row sensor is configured to produce a signal indicating a partial or full interruption in the first row beam received at the first row receiver caused by an object disposed between the first row emitter and the first row receiver, to produce a signal indicating an uninterrupted first row beam received at the first row receiver, or both.

In some embodiments, the foot pedal array is configured such that a foot positioned over and proximate to or on the first foot pedal or the second foot pedal produces a signal indicating a partial or full interruption in the first row beam.

In some embodiments, the foot pedal array is configured such that: a foot positioned over and proximate to or on the first foot pedal produces a signal from the first column sensor indicating a partial or full interruption of the first beam and a simultaneous signal from the first row sensor indicating a partial or full interruption of the first row beam; a foot positioned over or proximate to or on the third foot pedal produces a signal from the first column sensor indicating a partial or full interruption of the first beam and does not produce simultaneous signal from the first row sensor indicating a partial or full interruption of the first row beam; a foot positioned over and proximate to or on the second foot produces a signal from the second column sensor indicating a partial or full interruption of the second beam and produces a simultaneous signal from the first row sensor indicating a partial or full interruption of the first row beam; and a foot positioned over and proximate to or on the fourth foot pedal produces a signal from the second column sensor indicating a partial or full interruption of the second beam and does not produce a simultaneous signal from the first row sensor indicating a partial or full interruption of the first row beam.

In some embodiments, the foot pedal array further includes a second row sensor including: a second row emitter configured to direct a second row beam along the first foot pedal tier and over at least a portion of the first foot pedal and the second foot pedal, or along the second foot pedal tier and over at least a portion of the third foot pedal and the fourth foot pedal; and a second row receiver configured to receive the second row beam. In some embodiments, the second row beam is also directed over at least a portion of the fifth foot pedal, over at least a portion of the sixth foot pedal, or both.

In some embodiments, the foot pedal array further includes: a first sidewall at least partially defining an edge of the left foot portion of the foot pedal tray in the first foot pedal tier; and a second sidewall facing the first sidewall and at least partially an edge of the right foot portion of the foot pedal tray in the first foot pedal tier.

In some embodiments, the first row transmitter is disposed on or in or supported on one of the first sidewall or the second sidewall, and the first row receiver is disposed on or in or supported on the other of the first sidewall or the second sidewall.

In some embodiments, the foot pedal array includes a processor configured to receive one or more signals from the first column sensor, the second column sensor or both. In some embodiments, the first foot pedal pair and the second foot pedal pair are configured to generate signals for controlling an electrosurgery function of the surgical robotic system. In some embodiments, the third foot pedal pair is configured to generate signals for changing or controlling a mode of operation of the robotic assembly. In some embodiments, a signal generated by the fifth foot pedal changes or controls a travel mode of operation of the robotic assembly, and a signal generated by the sixth foot pedal changes or controls a camera motion mode of operation, which may also be referred to here as a view mode of operation, of the robotic assembly. In some embodiments, a signal generated by the fifth foot pedal changes or controls a translation mode of operation of the robotic assembly, and a signal generated by the sixth foot pedal changes or controls a pivot mode of operation of the robotic assembly.

In another aspect the present disclosure is directed to an operator console for receiving input from an operator to control a robotic assembly of a surgical robotic system, the operator console including: a foot pedal array disposed such that each of the first foot pedal, the second foot pedal, the third foot pedal, the fourth foot pedal, the fifth foot pedal, and the sixth foot pedal are accessible to one or both feet of the operator; and a plurality of controls manipulable by one or both hands of the operator.

In another aspect, an operator console for receiving input from an operator to control a robotic assembly of a surgical robotic system, the operator console including any of the foot pedal assemblies described herein.

In some embodiments, systems according to the present disclosure may increase the safety of surgical procedures by reducing the likelihood of a user inadvertently selecting the wrong foot pedal when the user approaches a foot pedal with a foot by identifying to a user which foot pedal the user is approaching with the foot before the user depresses the foot pedal. In some embodiments, systems according to the present disclosure may provide for increased ease of use by users.

As noted above, embodiments of the present disclosure provide a foot pedal array for receiving input from an operator to control a robotic assembly of a surgical robotic system, an operator console including such a foot pedal array, and surgical robotic systems including such a foot pedal array. Further, some embodiments of foot pedal arrays and operator consoles described herein may be employed with endoscopic surgical systems that are not robotic or that are only robotic in part.

A foot pedal array and a surgeon console or operator console employing a foot pedal array may be understood with reference to certain embodiments shown in FIG. 25 to FIG. 30.

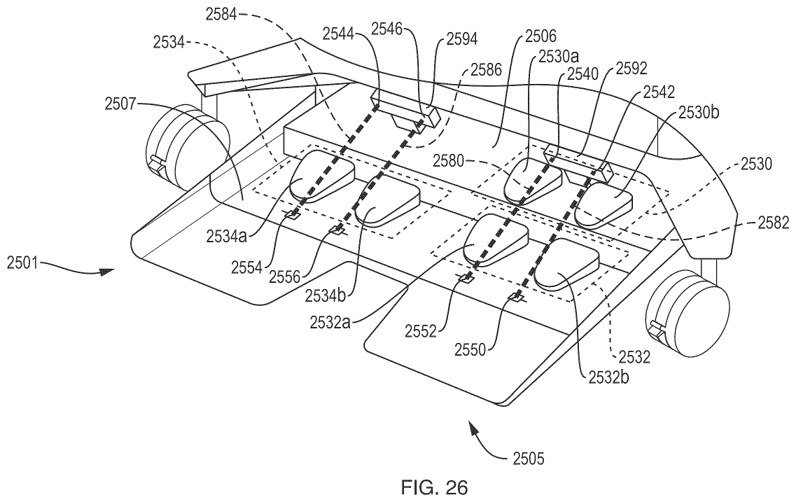

FIG. 25 depicts an operator console 2500 including a foot pedal array 2501 in accordance with some embodiments. FIG. 26 depicts a detailed view of the foot pedal array 2501 of the operator console 2500. The operator console 2500 also includes a cart 2520 that is provided with a cart base 2521, two hand controllers 2512, 2514, and a display device 2510 in accordance with some embodiments. The display device 2510 may be, for example, a monitor or screen. In other embodiments, the user may use a headset which may supplement or take the place of the display device 2510. In some embodiments, the cart base 2521 includes four locking wheels that permit the cart 2520 to be moved. The foot pedal array 2501 is disposed below the cart base 2521 in accordance with some embodiments. The foot pedal array 2501 may be attached to cart base 2521 directly, or suspended under the cart base 2521 in accordance with some embodiments. In some embodiments, the foot pedal assembly 2501 may be incorporated into the cart base or housed, at least in part, in the cart base. In some embodiments, the foot pedal array 2501 may be removably attached to the operator console 2500 to facilitate cleaning, repair, or replacement of the foot pedal array 2501 with a different foot pedal array as provided herein. In some embodiments, the operator console 2500, or portions of the operator console (e.g., the cart base 2521) may be provided separately from the foot pedal array 2501 and the foot pedal array 2501 may be removably attached or connected to the operator console 2500. The connection of the foot pedal array 2501 to the operator console 2500 may also be adjustable to permit different configurations to be selected by the user in accordance with some embodiments. The foot pedal array 2501 may be positioned slightly above the ground or floor to permit movement of operator console 2501 in accordance with some embodiments.

The foot pedal array 2501 includes a foot pedal tray 2505 having a first foot pedal tier 2506 and a second foot pedal tier 2507. The second foot pedal tier 2507 is disposed lower (e.g., closer to the floor) than the first foot pedal tier 2506 and forward relative to the first foot pedal tier 2506 so that the second foot pedal tier 2507 extends further toward the operator when the foot pedal array 2501 is positioned for use. The foot pedal tray 2505 may generally be described as having a left foot portion 2505L corresponding to a portion of the foot pedal tray more readily accessible by the operator's left foot than a right foot portion 2505R corresponding to a portion of the foot pedal tray more accessible to the operator's right foot when the operator is seated or standing facing the foot pedal array 2501. In some embodiments, the foot pedal tray 2505 includes a recess 2503 at a proximal end located near the position at which a user would stand or sit during use. The recess 2503 may facilitate the user accessing the foot pedal array 2501.

As shown in FIGS. 25 and 26, the foot pedal tray 2505 may be provided with a plurality of foot pedals including a first foot pedal pair 2530, a second foot pedal pair 2532, and a third foot pedal pair 2534 in accordance with some embodiments. The first foot pedal pair 2530 is located on the first foot pedal tier 2506 in the right foot portion 2505R of the foot pedal tray and includes a first foot pedal 2530a and a second foot pedal 2530b adjacent to the first foot pedal. The second foot pedal pair 2532 is located on the second foot pedal tier 2507 in the right foot portion 2505R of the foot pedal tray and includes a third foot pedal 2532a and a fourth foot pedal 2532b adjacent to the third foot pedal. The third foot pedal pair 2534 is located in the left foot portion 2505L of the foot pedal tray and includes a fifth foot pedal 2534a and a sixth foot pedal 2534b. In some embodiments, both the fifth foot pedal 2534a and the sixth foot pedal 2534b of the third foot pedal pair 2534 are located on the second foot pedal tier 2507 as shown. In other embodiments, the third foot pedal pair may be located on the first foot pedal tier of the foot pedal tray 2505. In other embodiments, the fifth foot pedal and the sixth foot pedal may be located on different foot pedal tiers of the foot pedal tray. The foot pedal array 2501 also includes a first column sensor that itself includes a first emitter 2540 and a first receiver 2550. The foot pedal array 2501 also includes a second column sensor that itself includes a second emitter 2542 and a second receiver 2552. In some embodiments, any or all of the column sensors may be photoelectric sensors. In some embodiments, any or all of the emitters for the sensors may light emitters, such as an infrared light emitter, in some embodiments. Any or all of the receivers may be light detector, such as a photoelectric detector or photodiode.

As illustrated, the emitters 2540 and 2542 are disposed at the first foot pedal tier 2506 distal to or beyond the first foot pedal pair 2530 and the receivers 2552 and 2550 are disposed at the second foot pedal tier 2507 proximal to the second foot pedal pair 2532; however, in some embodiments the positions may be reversed with the emitters disposed on the second foot pedal tier proximal to the second foot pedal pair and the receivers disposed at the first foot pedal tier distal to or beyond the first foot pedal pair. In some embodiments, both the transmitters and receivers are disposed at the first foot pedal tier distal to the first foot pedal pair and reflectors that reflect the emitted light back to the receivers are disposed at the second foot pedal tier proximal to the second foot pedal pair. In some embodiments, both the transmitters and receivers are disposed at the second foot pedal tier proximal to the second foot pedal pair and reflectors that reflect the emitted light back to the receivers are disposed at the first foot pedal tier distal to the first foot pedal pair. The term “column sensor” as used herein refers to a sensor and an emitter that directs a beam over a column of foot pedals in a generally forward/backward or proximal/distal direction. As shown in FIG. 26, the first column sensor and the second column sensor are configured so that a first beam 2580 emitted by the first emitter 2540 and a second beam 2582 emitted by the second emitter 2542 travel a respective column of foot pedals, specifically foot pedals 2530a and 2532b for the first column sensor and foot pedals 2530b and 2532a for the second column sensor, to the first receiver 2550 or the second receiver 2552, respectively. As shown in FIGS. 25 and 26, in some embodiments, each of the first emitter 2540 and the second emitter 2542 are disposed on a holder 2592 projecting from a base of the first foot pedal tier 2506. In some embodiments, the holder may be “T-shaped” as shown. In some embodiments, the first receiver 2552 and the second receiver 2550 are inset into in a base of the second foot pedal tier 2507.

In some embodiments, the first column sensor is configured to produce a signal indicating a partial or full interruption in the first beam 2580 received at the first receiver 2550 caused by an object (e.g., an operator foot) disposed between the first emitter 2540 and the first receiver 2550, to produce a signal indicating an uninterrupted first beam received at the first receiver 2550, or both. In some embodiments, the second column sensor is configured to produce a signal indicating a partial or full interruption in the second beam 2582 received at the second receiver 2552 caused by an object (e.g., an operator foot) disposed between the second emitter 2542 and the second receiver 2552, to produce a signal indicating an uninterrupted second beam received at the first receiver, or both.

In some embodiments, the foot pedal array 2501 is configured such that a foot positioned over and proximate to or on the first foot pedal 2530a or the second foot pedal 2530b produces a signal indicating a partial or full interruption in the first beam, and a foot positioned over and proximate to or on the third foot pedal 2532a or the fourth foot pedal 2532b produces a signal indicating a partial or full interruption in the first beam.

In some embodiments the foot pedal array 2501 also includes a third column sensor including a third emitter 2544 configured to direct a third beam 2584 over at least a portion of the fifth foot pedal 2534a as shown in FIG. 26. The third column sensor also includes a third receiver 2556. Although the emitter 2544 directs a beam over only one foot pedal, the sensor is still considered a “column” sensor because the beam is directed in a forward-backward or proximal-distal direction. In some embodiments in which the fifth foot pedal 2534a is on the same foot pedal tier as the sixth foot pedal 2534a, the foot pedal array 2501 may also include a fourth column sensor including a fourth emitter 2546 configured to direct a fourth beam 2586 over at least a portion of the fourth foot pedal 2534b as shown in FIG. 26. The fourth column sensor also includes a fourth receiver 2556. The features and aspects described herein with respect to embodiments of the first column sensor and second column sensor also apply to the third column sensor and the fourth column sensor.

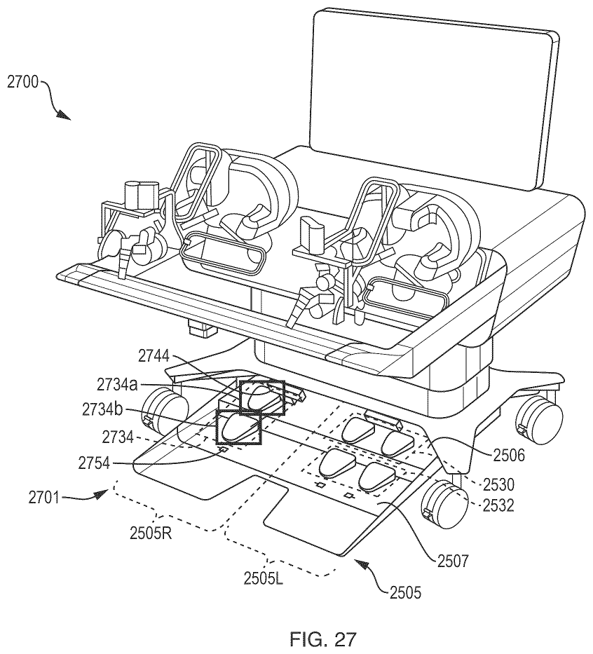

FIG. 27 depicts another embodiment of an operator console 2700. Features of the operator console 2700 that are similar to features of the operator console 2500 described above are identified with the same reference numbers for convenience. In a foot pedal assembly 2701 of the operator console 2700, a third foot pedal pair 2734 is positioned such that a fifth foot pedal 2734a is on the first foot pedal tier 2506 and a sixth foot pedal 2734b of the third foot pedal pair 2534 is on the second foot pedal tier 2507. As shown in FIG. 27, for the operator console 2700, a third column sensor includes a third emitter 2744 and a third receiver 2754. Because the fifth foot pedal 2734a and the sixth foot pedal 2734b are both in a same column, the third emitter 2744 directs a beam over at least a portion of both the fifth foot pedal 2734a and the sixth foot pedal 2734b. In such an embodiment, one column sensor may be used for the third foot pedal pair 2734 instead of two column sensors.

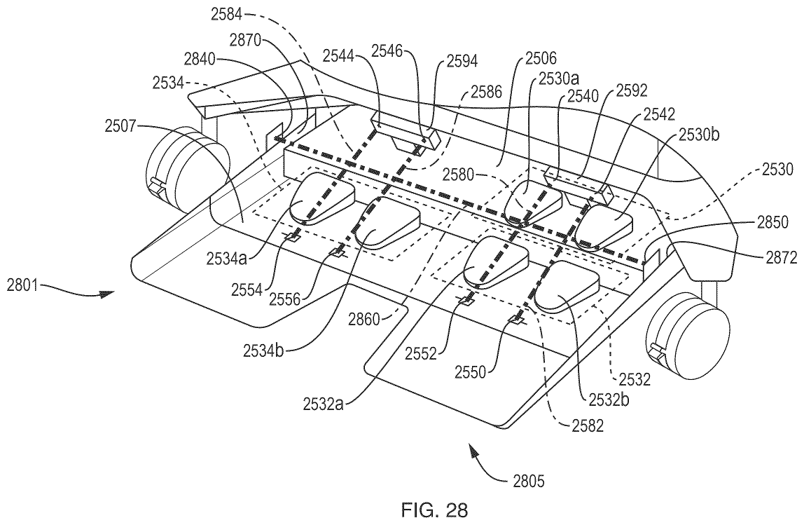

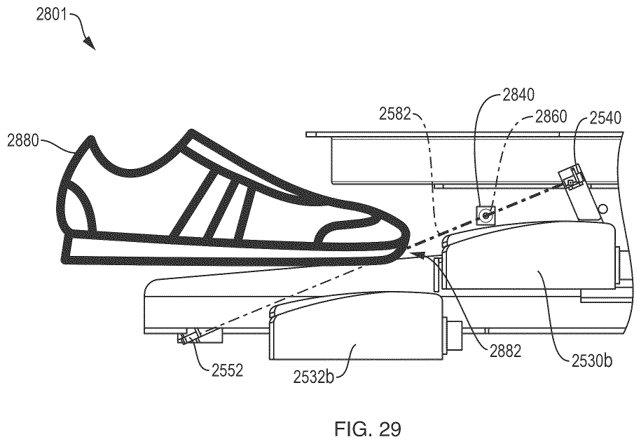

FIGS. 28 and 29 depict a foot pedal array 2801 in accordance with another embodiment. With respect to arrangements of a first pair of foot pedals 2530, a second pair of foot pedals 2532, a third set of foot pedals 2534 and a corresponding first column sensor, second column sensor, third column sensor, and fourth column sensor, the features of foot pedal array 2801 and foot pedal tray 2805 are similar to those of the foot pedal array 2501 described above. The foot pedal array 2801 also includes a first row sensor including a first row emitter 2840 configured to direct a first row beam 2860 laterally along a second foot pedal tier 2507 and over at least a portion of the third foot pedal 2532a and the fourth foot pedal 2532b. The first row sensor also includes a first row receiver 2850 configured to receive the first row beam 2850. In embodiments in which the fifth foot pedal 2534a and sixth foot pedal 2534b are also on the second foot pedal tier 2507, the first row beam 2850 is directed over at least a portion of the fifth foot pedal 2534a and sixth foot pedal 2534b as well. The term row may be used herein to refer to an axis running perpendicular to a column, such as laterally from the left foot portion of the foot pedal array 2501 to the right foot portion of the foot pedal array 2501, or vice versa.

As explained above with respect to the embodiments in FIGS. 25 and 26, the first column sensor is configured to produce a signal indicating a partial or full interruption in the first beam 2580 received at the first receiver 2550 caused by an object (e.g., an operator foot) disposed between the first emitter 2540 and the first receiver 2550, to produce a signal indicating an uninterrupted first beam received at the first receiver 2550, or both. The second column sensor is configured to produce a signal indicating a partial or full interruption in the second beam 2582 received at the second receiver 2552 caused by an object (e.g., an operator foot) disposed between the second emitter 2542 and the second receiver 2552, to produce a signal indicating an uninterrupted second beam 2582 received at the second receiver 2552, or both. The third column sensor is configured to produce a signal indicating a partial or full interruption in the third beam 2584 received at the third receiver 2554 caused by an object (e.g., an operator foot) disposed between the third emitter 2544 and the third receiver 2554, to produce a signal indicating an uninterrupted third beam 2584 received at the third receiver 2554, or both. The fourth column sensor is configured to produce a signal indicating a partial or full interruption in the fourth beam 2586 received at the fourth receiver 2556 caused by an object (e.g., an operator foot) disposed between the fourth emitter 2546 and the third receiver 2556, to produce a signal indicating an uninterrupted second beam 2586 received at the second receiver 2554, or both. In the foot pedal assembly 2801 of FIGS. 28 and 29, the first row sensor is configured to produce a signal indicating a partial or full interruption in the first row beam 2860 received at the first row receiver 2850 caused by an object disposed between the first row emitter 2840 and the first row receiver 2850, to produce a signal indicating an uninterrupted first row beam 2860 received at the first row receiver 2850, or both. The additional row sensor in combination with the first, second, third, and fourth beam sensors enables the foot pedal array 2801 to identify which individual foot pedal has an object (e.g., a foot 2880) over it and proximate to it or on it or on it based on which beams are interrupted or which beams are not interrupted.

For example, in some embodiments with the configuration depicted in FIG. 28, a foot positioned over and proximate to or on the first foot pedal 2530a produces a signal from the first column sensor indicating a partial or full interruption of the first beam 2580 and a simultaneous signal from the first row sensor indicating a partial or full interruption of the first row beam 2860. A foot positioned over or proximate to and on the third foot pedal 2532a produces a signal from the first column sensor indicating a partial or full interruption of the first 2580 beam and does not produce simultaneous signal from the first row sensor indicating a partial or full interruption of the first row beam 2860. A foot positioned over and proximate to or on the second foot pedal 2530b produces a signal from the second column sensor indicating a partial or full interruption of the second beam 2582 and produces a simultaneous signal from the first row sensor indicating a partial or full interruption of the first row beam 2860. A foot positioned over and proximate to or on the fourth foot pedal 2532b, as illustrated in FIG. 29, produces a signal from the second column sensor indicating a partial or full interruption 2882 of the second beam 2582 and does not produce a simultaneous signal from the first row sensor indicating a partial or full interruption of the first row beam 2860. A foot positioned over and proximate to or on the fifth foot pedal 2534a produces a signal from the third column sensor indicating a partial or full interruption of the third beam 2586 and does not produce a simultaneous signal from the first row sensor indicating a partial or full interruption of the first row beam 2860. A foot positioned over and proximate to or on the sixth foot pedal 2534b produces a signal from the third column sensor indicating a partial or full interruption of the fourth beam 2588 and does not produce a simultaneous signal from the first row sensor indicating a partial or full interruption of the first row beam 2860.

In some embodiments the first row sensor may disposed at the second foot pedal tier instead of the first foot pedal tier. In some embodiments, a first row sensor may be disposed on a first foot pedal tier and a second row sensor may be disposed on a second foot pedal tier. In some embodiments in which the fifth foot pedal and sixth foot pedal are on different foot pedal tiers and in a single column, a row sensor may be used to distinguish between an object over the fifth foot pedal and an object over the sixth foot pedal.

In some embodiments, the first row emitter 2840 is in a left portion 2505L of the foot pedal tray and the first row receiver 2850 is in a right portion 2505R of the foot pedal tray. In some embodiments, the first row emitter is in a right portion of the foot pedal tray and the first row receiver is in a left portion of the foot pedal tray. In some embodiments, the foot pedal tray includes a left sidewall 2870 and a right sidewall 2872 facing the left sidewall, and the first row emitter 2840 and the first row receiver 2850 are attached to, extend from, or are mounted to the left sidewall 2870 or the right sidewall 2872. In some embodiments, the first sidewall (e.g., left sidewall 2870) at least partially defines and edge of the left foot portion of the foot pedal tray 2805 in the first foot pedal tier 2507. In some embodiments, the second sidewall (e.g., right sidewall 2872) at least partially defies an edge of the right foot portion of the foot pedal tray 2805 in the first foot pedal tier 2506. In some embodiments, both the first row emitter and the first row receiver are disposed on a same side of the foot pedal tray, and a reflector is disposed on an opposite side of the foot pedal tray to reflect the emitted beam back to the receiver.

### Functions of the Foot Pedals in the Surgical Robotic System

The foot pedals described herein may be used to operate or control a variety of functions of the surgical robotic system in accordance with some embodiments. For example, the first foot pedal pair 2530 and/or the second foot pedal pair 2532 may be configured to generate signals for controlling at least one electrosurgery function of the surgical robotic system. In some embodiments, the second foot pedal pair 2532 may constitute secondary electrosurgical foot pedals and the first foot pedal pair 2530 may constitute primary electrosurgical foot pedals, or vice versa. In some embodiments, the third foot pedal pair 2534 may be configured to generate signals for changing or controlling a mode of operation (or functions) of the robotic assembly. In some embodiments, a signal generated by the fifth foot pedal 2534a or a signal generated by the sixth foot pedal 2534b may change or control a mode of operation or a mode of control of the robotic assembly from a default mode, in which movement of at least one hand controller causes a corresponding movement of a corresponding robotic arm, to a different mode controlling motions, such as a travel mode in which movement of at least one hand controller can cause a movement (e.g., a change in orientation or in translation) of a base of a robotic arm or a virtual chest of the robotic assembly while maintaining a view of the camera assembly centered on an instrument tip of the robotic arm where the robotic assembly has only one robotic arm, or on a position between instrument tips of instrument arms where the robotic assembly has two robotic arms. In some embodiments, a movement of one of the hand controllers when the travel mode is activated causes a corresponding movement of an instrument tip of a corresponding robotic arm relative to the view of the camera assembly, causes the camera assembly to rotate to center the view of the camera assembly on the average tip position, and causes a virtual chest of the robotic assembly to be translated, to be rotated, or both to maintain a distance between a center of the virtual chest of the robotic assembly and the average instrument tip position within an acceptable distance range and to maintain an angular deviation between a line from the center of the virtual chest to the average instrument tip position and a normal to the virtual chest within an acceptable angular deviation range. In some embodiments, while in the travel mode, the chest of the robotic assembly and/or the camera assembly automatically translates or reorients to maintain a configuration the robotic assembly had upon entering the travel mode.

In some embodiments, a signal generated by the other of the sixth foot pedal 2534b or the fifth foot pedal 2534a may change or control a mode of operation (or functions) or a mode of control (or articulation) of the robotic assembly from a default mode in which movement of at least one hand controller causes a corresponding movement of a robotic arm to a camera mode, also referred to herein as a view mode, in which movement of at least one hand controller changes an orientation and/or position of a camera assembly without changing a position or an orientation of the at least one robotic arm. In some embodiments, the different mode is a view mode in which a movement of one of the hand controllers causes a corresponding change in an orientation of the camera assembly and in a position of the camera assembly due to a translation of the virtual chest, and a rotation of the virtual chest of the robotic assembly to maintain an angular deviation between a line from the center of the virtual chest to a position of a midpoint between instrument tips of robotic arms of the robotic assembly, which is an average instrument tip position while maintaining positions and orientations of instrument tips of all robotic arms of the robotic assembly.

In some embodiments, the different mode is a translate mode in which a movement of one of the hand controllers causes a corresponding movement of an instrument tip of a corresponding robotic arm relative to the view of the camera assembly, causes rotation of the camera assembly to center the view of the camera assembly on the average tip position, and causes translation of the virtual chest to maintain a distance between the center of the virtual chest and the average instrument tip position within in an acceptable distance range. In some embodiments, the translate mode maintains the robotic assembly in a configuration and orientation that it had upon entering the translate mode.

In some embodiments, the different mode is a pivot mode in which a movement of one of the hand controllers causes a corresponding movement of an instrument tip of a corresponding robotic arm relative to the view of the camera assembly and causes a change in the orientation of the camera assembly or a change in the orientation of the camera assembly and a change in the orientation of the virtual chest to maintain the view of the camera assembly centered on the average instrument tip position and to maintain an angular deviation between a line from the center of the virtual chest to the average instrument tip position and a normal to the virtual chest within an acceptable angular deviation range. Further disclosure regarding different modes of operation and control can be found in the provisional application entitled “Hand Controllers and Control Modes for a Robotic Surgical System,” which is filed on the same day at the present application and is incorporated herein in its entirety.

In some embodiments, when an object, such as an operator's foot, interrupts one or more beams of a foot pedal assembly, one or more signals from the first column sensor, the second column sensor, the third column sensor, the fourth column sensor and/or at least one row sensor may cause a surgical robotic system to display information in a graphical user interface regarding a function of a foot pedal that the operator's foot is currently over or on. For example, FIG. 30 schematically depicts a graphical user interface (GUI) 3000 for an operator including a central area for displaying an image based on image input from the camera assembly. The GUI can include one or more portions, e.g., first information portion 3020 and second information portion 3030, that display information that may include any information regarding a control mode, an operation mode, a current configuration of the robotic assembly, and foot pedal selection information. In some embodiments, at least one information portion includes foot pedal selection information. For example, the first information portion 3020 may include first foot pedal group selection information 3022 where the first foot pedal group includes the third foot pedal pair, and the foot pedal selection information indicates which foot pedal the user's foot is over or on, and/or the function of the foot pedal that the user's foot is over or on. In some embodiments, the second information portion 3030 includes second foot pedal group selection information 3032. In some embodiments, the first foot pedal group includes the first foot pedal pair and the second foot pedal pair, and the foot pedal selection information indicates which foot pedal the user's foot is over or on, and/or the function of the foot pedal that the user's foot is over or on. In some embodiments, the first foot pedal group selection information and the second foot pedal group selection information may be included in the same information portion of the GUI. In some embodiments, the second foot pedal group includes the first foot pedal pair and the second foot pedal pair is included in a third foot pedal group disposed in a third information portion of the GUI.

While some embodiments of the present disclosure have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. One or more hand controllers for controlling a surgical robotic system including a robotic assembly configured to be disposed with an internal body cavity of a subject, each of the one or more hand controllers comprising:
a contoured housing having an upper surface, an inside side surface adjacent the upper surface, an outside side surface facing away from the inside side surface, and a lower surface facing away from the upper surface;
a plurality of buttons including a first button positioned on the upper surface and a second button positioned on one of the upper surface, the inside side surface, or the outside side surface;
a first touch input device positioned on the upper surface; and
a first paddle disposed at either of the inside side surface or the outside side surface;
wherein the contoured housing is configured to be grasped with a user's thumb on the inside side surface and at least some of the user's fingers, the user's palm, or both over the upper surface, and the first button is configured to be manipulated by the user's pointer finger or index finger, and
wherein for at least one of the hand controllers, at least one of the plurality of buttons maps to a function selected from one or more of the following functions:
resetting a pose of the surgical robotic assembly with respect to a plurality of robotic arms and a virtual chest of the robotic assembly to a default pose while maintaining a position and an orientation of an instrument tip for each of the plurality of robotic arms; or
activating or exiting a view mode of the surgical robotic system, wherein activating the view mode causes a change in an orientation of a camera assembly or causes in the orientation of the camera assembly and a change in an orientation of the virtual chest to center a view of the camera assembly on a position of a midpoint between instrument tips of the plurality of robotic arms, which is an average instrument tip position, and a movement of one of the hand controllers when the view mode is activated causes a corresponding movement of an instrument tip of a corresponding one of the plurality of robotic arms relative to a view of the camera assembly and causes a change in the orientation of the camera assembly or the change in the orientation of the camera assembly and the change in the orientation of the virtual chest to maintain a view of the camera assembly centered on the average instrument tip position.

2. The one or more hand controllers of claim 1, wherein for each of the one or more hand controllers, a contact surface of the first paddle is disposed at the outside side surface of the housing.

3. The one or more hand controllers of claim 1, each of the one or more hand controllers further comprising a second paddle, wherein a contact surface of the first paddle is disposed at the outside side surface of the housing and a contact surface of the second paddle is disposed at the inside side surface of the housing, or the contact surface of the first paddle is disposed at the inside side surface of the housing and the contact surface of the second paddle is disposed at the outside side surface of the housing.

4. The one or more hand controllers of claim 3, wherein the first paddle is engaged with the second paddle via one or more gears, and wherein a movement of the first paddle causes a reciprocal movement of the second paddle, and a movement of the second paddle causes a reciprocal movement of the first paddle.

5. The one or more hand controllers of any one of claims 1 to 4, wherein for each of the hand controllers, the first touch input device is a scroll wheel, a rocker button, a joy stick, a pointing stick, a touch pad, or a trackball.

6. The one or more hand controllers of any one of claims 1 to 5, wherein at least one of the one or more hand controllers further comprises a second touch input device.

7. The one or more hand controllers of any one of claims 1 to 6, wherein for each of the hand controllers, deflection of the first paddle is configured to produce a signal that the surgical robotic system uses as an input to control the robotic subassembly.

8. The one or more hand controllers of any one of claims 1 to 7, wherein for at least one of the hand controllers, at least one of the plurality of buttons maps to a function selected from one or more functions of the following functions:
triggering activation of a clutch with respect to a hand controller, the activation of the clutch permitting or enabling movement of the respective hand controller without causing movement of the robotic assembly; or
activating or exiting an instrument control mode of the surgical robotic system, a movement of one of the hand controllers when in the instrument mode causing a corresponding movement in a corresponding robotic arm of the robotic assembly; or
activating or exiting a scan mode of the surgical robotic system, a movement of one of the hand controllers when in the scan mode causing a corresponding change in an orientation of a camera assembly of the robotic assembly while the robotic arm of the robotic assembly remains stationary where the robotic assembly includes one robotic arm, or while all robotic arms of the robotic assembly remain stationary where the robotic assembly includes more than one robotic arm; or
activating or exiting a menu feature of the surgical robotic system, the menu feature causing a menu to be displayed in a graphical user interface of the surgical robotic system and input from a touch input device or a button of the one or more hand controllers being used for selection of an item of the menu; or
navigating between and/or selecting options from the menu displayed in the graphical user interface of the surgical robotic system; or
activating or exiting an elbow bias feature, the elbow bias feature enabling adjustment of a magnitude and a direction of an elevation of an elbow of a robotic arm while maintaining a position of an instrument tip of the robotic arm for each robotic arm of the robotic assembly; or
controlling an adjustment of an elevation of an elbow of a robotic arm of the robotic assembly for one robotic arm of the robotic assembly or for each robotic arm of the robotic assembly; or
changing or selecting a zoom of a view from a camera assembly of the robotic assembly.

9. The one or more hand controllers of claim 8, wherein for at least one of the hand controllers, at least two of the plurality of buttons map to functions selected from the one or more functions.

10. The one or more hand controllers of claim 8, wherein for at least one of the hand controllers, at least three of the plurality of buttons maps to functions selected from the one or more functions.

11. The one or more hand controllers of any one of claims 8 to 10, wherein for at least one of the one or more hand controllers, the first touch input device has one or more functions selected from the one or more functions.

12. The one or more hand controllers of any one of claims 8 to 11, wherein at least one of the one or more hand controllers further comprises a second touch input device; and
wherein for at least one of the one or more hand controllers, the first touch input device or the second touch input device has one or more functions selected from the one or more functions.

13. The one or more hand controllers of claim 12, wherein at least one of the one or more hand controllers further comprises a second touch input device; and
wherein for at least one of the one or more hand controllers, the first touch input device and the second touch input device each have one or more functions selected from the one or more functions.

14. The one or more hand controllers of any one of claims 1 to 13, wherein for at least one of the one or more hand controllers, an engagement of the first button produces a signal triggering activation of a clutch feature of the surgical robotic system with respect to a hand controller, and wherein activation of the clutch feature permits or enables movement of the respective hand controller without causing movement of the robotic assembly.

15. The one or more hand controllers of any one of claims 1 to 14, wherein for each hand controller, the outside side surface of the housing is substantially concave.

16. The one or more hand controllers of any one of claims 1 to 15, each of the one or more hand controllers further comprising a thumb pad disposed on either of the inside side surface or the outside side surface.

17. The one or more hand controllers of any one of claims 1 to 16, at least one of the one or more hand controllers including a sensor configured to sense contact of a hand of the operator with the hand controller or to sense proximity of a hand of the operator to the hand controller.

18. The one or more hand controller of claim 17, wherein an input from the sensor is used for activating or exiting an instrument control mode of the surgical robotic system, a movement of one of the hand controllers when in the instrument mode causing a corresponding movement in a corresponding robotic arm of the robotic assembly.

19. One or more hand controllers for controlling a surgical robotic system including a robotic assembly configured to be disposed with an internal body cavity of a subject, each of the one or more hand controllers comprising:
a contoured housing having an upper surface, an inside side surface adjacent the upper surface, an outside side surface facing away from the inside side surface, and a lower surface facing away from the upper surface;
a plurality of buttons including a first button positioned on the upper surface and a second button positioned on one of the upper surface, the inside side surface, or the outside side surface;
a first touch input device positioned on the upper surface;
a first paddle disposed at the outside side surface; and
a second paddle disposed at the inside side surface;
wherein the contoured housing is configured to be grasped with a user's thumb on the inside side surface and at least some of the user's fingers, the user's palm, or both over the upper surface, and the first button is configured to be manipulated by the user's pointer finger or index finger.

20. A hand controller system for controlling a surgical robotic system including a robotic assembly configured to be disposed within an internal body cavity of a subject, the hand controller system comprising:
a first hand controller as set forth in any one of claims 1 to 20 removably coupled to or configured to be connected to an operator console via a first hand controller support;
a second hand controller as set forth in any one of claims 1 to 20 removably coupled to or configured to be connected to the operator console via a second hand controller support, wherein an inside surface of the first hand controller faces an inside surface of the second hand controller when the first and second hand controllers are in a neutral position in use.

21. A hand controller system for controlling a surgical robotic system including a robotic assembly configured to be disposed with an internal body cavity of a subject, the hand controller system comprising:
a first hand controller removably coupled to or configured to be connected to an operator console via a first hand controller support, the first hand controller comprising:
a contoured housing having an upper surface, a first side surface adjacent the upper surface, a second side surface facing away from the first side surface, and a lower surface facing away from the upper surface;
a first button and a first scroll wheel disposed at the upper surface;
a first paddle having a contact surface disposed at the first side surface or at the second side surface; and
a second button disposed at the first side surface or at the second side surface; and
a second hand controller removably coupled to or configured to be connected to the operator console via a second hand controller support, the second hand controller comprising:
a contoured housing having an upper surface, a first side surface adjacent the upper surface, a second side surface facing away from the first side surface, and a lower surface facing away from the upper surface;
a first button and a first scroll wheel disposed at the upper surface;
a first paddle having a contact surface disposed at the first side surface or at the second side surface; and
a second button disposed at the first side surface or at the second side surface, wherein an inside surface of the first hand controller faces an inside surface of the second hand controller when the first and second hand controllers are in a neutral position in use.

22. A surgeon console comprising:
the hand controller system of claim 1, claim 19 or claim 20; and
a plurality of foot pedals including one or more of:
a foot pedal configured to activate a view mode of the surgical robotic system upon engagement, a movement of one of the hand controllers when the view mode is activated causing a corresponding change in an orientation of and a position of the camera assembly and a rotation of the virtual chest of the robotic assembly to maintain an angular deviation between a line from the center of the virtual chest to a position of a midpoint between instrument tips of robotic arms of the robotic assembly, which is an average instrument tip position, and the normal to the virtual chest within an acceptable angular deviation range while maintaining positions and orientations of instrument tips of all robotic arms of the robotic assembly,
wherein activating the view mode further causes a change in an orientation of the camera assembly or a change in the orientation of the camera assembly and a change in an orientation of a virtual chest of the robotic assembly to center a view of the camera assembly on a position of a midpoint between instrument tips of robotic arms of the robotic assembly, which is an average instrument tip position, and a movement of one of the hand controllers when the view mode is activated causing a corresponding movement of an instrument tip of a corresponding robotic arm relative to the view of the camera assembly and causing a change in the orientation of the camera assembly or a change in the orientation of the camera assembly and a change in the orientation of the virtual chest to maintain the view of the camera assembly centered on the average instrument tip position and to maintain an angular deviation between a line from the center of the virtual chest to the average instrument tip position and a normal to the virtual chest within an acceptable angular deviation range;
a foot pedal configured to activate a travel mode of the surgical robotic system upon engagement, a movement of one of the hand controllers when the travel mode is activated causing a corresponding movement of an instrument tip of a corresponding robotic arm by a corresponding movement including a corresponding translation and a corresponding rotation relative to the view of the camera assembly, causing the camera assembly to rotate to center the view of the camera assembly on the average tip position, and causing a virtual chest of the robotic assembly to be translated, to be rotated, or both to maintain a distance between a center of the virtual chest of the robotic assembly and the average instrument tip position within an acceptable distance range and to maintain an angular deviation between a line from the center of the virtual chest to the average instrument tip position and a normal to the virtual chest within an acceptable angular deviation range; and
a foot pedal configured to activate a translate mode of the surgical robotic system upon engagement, a movement of one of the hand controllers when the transverse mode is activated causing a corresponding movement of an instrument tip of a corresponding robotic arm relative to the view of the camera assembly, causing rotation of the camera assembly to center the view of the camera assembly on the average tip position, and causing translation of the virtual chest to maintain a distance between the center of the virtual chest and the average instrument tip position within in an acceptable distance range.

23. A method for controlling a robotic assembly of a surgical robotic system, the surgical robotic system comprising an image display and right hand controller and a left hand controller configured to sense a movement of an operator's hands, the robotic assembly including a camera assembly and a robotic arm assembly including a first robotic arm and a second robotic arm each of the first robotic arm and the second robotic arm having an associated instrument tip, positions and orientations of the robotic arms and camera assembly defining a virtual chest of the robotic assembly, the method comprising:
while the robotic assembly is disposed in an interior cavity of a subject, receiving a first control mode selection input and changing a default control mode of the surgical robotic system to a first control mode in response to the first control mode selection input, the surgical robotic system having a plurality of control modes including one or more of: a scan mode, a view mode, a travel mode, and a translation mode, and the first control mode being the scan mode, the view mode, the travel mode, or the translation mode; and
in response to a first movement including a first translation and a first rotation of the right hand controller or the left hand controller while the robotic assembly is in the first control mode:
where the first control mode is the scan mode, holding the robotic arm assembly stationary while rotating the camera assembly in a corresponding first rotation relative to a view of the camera assembly displayed on the image display of the surgical robotic system, which is a displayed camera view;
where the first control mode is the travel mode, moving the corresponding instrument tip by a corresponding first movement including a corresponding first translation and a corresponding first rotation relative to the displayed camera view while rotating the camera assembly to center the view of the camera assembly on a position of a midpoint between instrument tips of the robotic arms of the robotic assembly, which is an average tip position, while translating the robotic assembly to translate a chest point of the virtual chest, rotating the robotic assembly to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range;
where the first control mode is the view mode, holding a position and an orientation of each instrument contestant while moving the camera assembly by a corresponding first movement including a corresponding first translation of the virtual chest and a corresponding first rotation of the camera assembly relative to the displayed camera view while rotating a virtual chest of the robotic assembly to maintain an angular deviation between the line from the center of the virtual chest to the average instrument tip position and the normal to the virtual chest within the acceptable angular deviation range, moving a corresponding instrument tip by a corresponding first movement including a corresponding first rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range; and
where the first control mode is the translate mode, moving a corresponding instrument tip by a corresponding first movement including a corresponding first translation and a corresponding first rotation relative to the displayed camera view while rotating the camera assembly or rotating the camera assembly and translating the virtual chest to center the view of the camera assembly on the average tip position and to maintain a distance between the center of the virtual chest and the average instrument tip position within the acceptable distance range.

24. The method of claim 23, wherein the current control mode is an instrument control mode in which a movement of the right hand controller or the left hand controller causes a corresponding movement of an instrument tip at a distal end of a corresponding robotic arm relative to the displayed camera view without moving or changing an orientation of the virtual chest of the robotic assembly.

25. The method of claim 23 or 24, wherein the first control mode selection input is received via a foot pedal of the surgical robotic system.

26. The method of claim 23 or 24, wherein the first control mode selection input is received via the first hand controller or the second hand controller.

27. The method of claim 26, wherein the first control mode selection input is received via a button or touch input device of the right hand controller or the left hand controller.

28. The method of any one of claims 23 to 27, wherein the first control mode is the view mode.

29. The method of any one of claims 23 to 27, wherein the first control mode is the scan mode.

30. The method of any one of claims 23 to 27, wherein the first control mode is the travel mode.

31. The method of any one of claims 23 to 27, wherein the first control mode is the transverse mode.

32. The method of any one of claims 23 to 27, wherein the first control mode is the view mode.

33. The method of any one of claim 23 to 32, further comprising:
receiving a second control mode selection input;
changing a current control mode of the surgical robotic system to a second control mode different than the first control mode in response to the second control mode selection input, wherein the plurality of control modes of the surgical robotic system includes two or more of the scanning mode, the view mode, the travel mode, and the translate mode; and
in response to a second movement including a second translation and a second rotation of the right hand controller or the left hand controller while the current control mode is the second control mode:
where the second control mode is the scan mode, holding the robotic arm assembly stationary while rotating the camera assembly in a corresponding second rotation relative to the displayed camera view;
where the second control mode is the travel mode, moving the corresponding instrument tip by a corresponding second movement including a corresponding second translation and a corresponding second rotation relative to the displayed camera view while rotating the camera assembly to center the view of the camera assembly on the an average tip position, while translating the robotic assembly to translate the chest point of the virtual chest, rotating the robotic assembly to rotate the virtual chest, or both, to maintain the distance between the center of the virtual chest and the average tip position in the acceptable distance range, and to maintain an angular deviation between the line from the chest point to the average tip position and the normal to the virtual chest within the acceptable angular deviation range;
where the second control mode is the view mode, holding a position and an orientation of each instrument while moving the camera assembly by a corresponding second movement including a corresponding second translation and a corresponding second rotation relative to the displayed camera view and rotating the virtual chest of the robotic assembly to maintain the angular deviation between the line from the center of the virtual chest to the average instrument tip position and the normal to the virtual chest within the acceptable angular deviation range, moving a corresponding instrument tip by a corresponding second movement including a corresponding second rotation relative to the displayed camera view, while rotating the camera assembly to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range; and
where the second control mode is the translate mode, moving a corresponding instrument tip by a corresponding second movement including a corresponding second translation and a corresponding second rotation relative to the displayed camera view while rotating the camera assembly or rotating the camera assembly and translating the virtual chest to center the view of the camera assembly on the average tip position and to maintain the distance between the center of the virtual chest and the average instrument tip position within the acceptable distance range.

34. The method of any one of claims 23 to 33, wherein the virtual chest is defined by a chest plane extending between a first pivot point of a most proximal joint of the first robotic arm, a second pivot point of a most proximal joint of the second robotic arm, and a camera imaging center point of the camera assembly when the camera is in a default position with respect to plunge.

35. The method of any one of claims 23 to 34, further comprising:
receiving a first function selection input corresponding to an input function located on the left hand controller, the right hand controller, or both; and
in response to receipt of the first function selection input, performing a first function.

36. The method of claim 35, wherein performing the first function comprises:
displaying a menu on a portion of the image display while holding positions and orientations of the camera assembly and the robotic arm assembly constant;
resetting a camera assembly orientation and position to align a camera view to an average instrument tip position and to the virtual chest; or
activating a clutch with respect to hand controller to enable movement of the respective hand controller without causing movement of the robotic assembly.

37. The method of claim 35 or 36, wherein the first function comprises displaying the menu on the portion of the image display while holding positions and orientation of the camera assembly and the robotic arm assembly constant; and wherein the method further comprises:
receiving one or more second function selection inputs corresponding to one or more input functions located on the left hand controller, the right hand controller, or both; and
in response receiving the one or more second function selection inputs, graphically indicating options on a portion of the image display for one or more menu item to be selected one a based on the one or more second function inputs, displaying one or more submenus on the portion of the image display corresponding to the one or more second function inputs.

38. The method of claim 37, further comprising:
receiving at least one third feature selection input corresponding to an input function located on the left hand controller, the right hand controller, or both, the at least one third feature selection input selecting an option on the image display; and
where the at least one third feature selection input selects a zoom level, in response receipt of the at least one third feature selection input, changing a zoom level of the displayed camera view and storing information regarding the new zoom level for modification of control modes that depend on zoom level; or
where the at least one third feature selection input selects an elbow bias level, or selects an elbow bias menu and includes a selection of an elbow bias level from the elbow bias menu, in response receipt of the third feature selection input, changing an elbow bias of a first robotic arm, of a second robotic arm, or of both.

39. A non-transitory computer readable medium storing instructions that, when executed by one or more processors of a robotic surgical system perform the method of any one of claims 23 to 38.

40. A surgical robotic system for performing a surgery within an internal cavity of a subject, the surgical robotic system comprising:
a right hand controller and a left hand controller operated to manipulate the surgical robotic system;
a camera assembly;
a robotic arm assembly configured to be inserted into the internal cavity during use, the robotic arm assembly including:
a first robotic arm including or coupled to a first instrument tip disposed at a distal end of the first robotic arm;
a second robotic arm including or coupled to a second instrument tip disposed at a distal end of the second robotic arm;
an image display for outputting an image from the camera assembly; and
at least one computing module or control unit configured to:
receive movement data from the right hand controller and the left hand controller and in response generate control signals based on a current control mode of the surgical robotic system;
receive a control mode selection input and in response change the current control mode of the surgical robotic system to a selected one of a plurality of control modes of the surgical robotic system;
receive a function selection input from the first hand controller and/or the left hand controller and generate control signals to perform a corresponding function of a plurality of functions.

41. The surgical robotic system of claim 40, wherein the plurality of control modes includes one or more of: a scan mode, a view mode, a travel mode, and a translation mode; and
wherein while the current control mode is the scan mode, in response to a first movement including a first rotation of the right hand controller or the left hand controller, the robotic arm assembly is held stationary while the camera assembly is rotated in a corresponding first rotation relative to a view of the camera assembly displayed on the image display, which is a displayed camera view;
wherein while the current control mode is the travel mode, in response to a first movement including a first translation and a first rotation of the right hand controller or the left hand controller, a corresponding instrument tip is moved by a corresponding first movement including a corresponding first translation and a corresponding first rotation relative to the displayed camera view while the camera assembly is rotated to center the view of the camera assembly on a position of a midpoint between instrument tips of the robotic arms of the robotic assembly, which is an average tip position, while robotic assembly is translated to translate a chest point of the virtual chest, robotic assembly is rotated to rotate the virtual chest, or both, to maintain a distance between the center of the virtual chest and the average tip position in an acceptable distance range, and to maintain an angular deviation between a line from the chest point to the average tip position and a normal to the virtual chest within an acceptable angular deviation range;
wherein while the current control mode is view mode, in response to a first movement including a first translation and a first rotation of the right hand controller or the left hand controller, a position and an orientation of each instrument is held while the camera assembly is moved by a corresponding first movement including a corresponding first translation and a corresponding first rotation relative to the displayed camera view and a virtual chest of the robotic assembly is rotated to maintain an angular deviation between the line from the center of the virtual chest to the average instrument tip position and the normal to the virtual chest within the acceptable angular deviation range, wherein a corresponding instrument tip is moved by a corresponding first movement including a corresponding first rotation relative to the displayed camera view, the camera assembly is rotated to center the view of the camera assembly on the average instrument tip position causing a change in the orientation of the camera assembly or a change in the orientation of the camera assembly and a change in the orientation of the virtual chest to maintain the view of the camera assembly centered on the average instrument tip position and to maintain an angular deviation between the line from the center of the virtual chest to the average instrument tip position and the normal to the virtual chest within the acceptable angular deviation range; and
wherein while the current control mode is the translate mode, in response to a first movement including a first translation and a first rotation of the right hand controller or the left hand controller, the corresponding instrument tip is moved by a corresponding first movement including a corresponding first translation and a corresponding first rotation relative to the displayed camera view while camera assembly is rotated or the camera assembly is rotated and the virtual chest is translated to center the view of the camera assembly on the average tip position and to maintain a distance between the center of the virtual chest and the average instrument tip position within the acceptable distance range.

42. The surgical robotic system of claim 41, in which the plurality of modes further comprises an instrument control mode in which a movement of the right hand controller or the left hand controller causes a corresponding movement of an instrument tip at a distal end of a corresponding robotic arm relative to the displayed camera view without moving or changing an orientation of the virtual chest of the robotic assembly.

43. The surgical robotic system of claim 41 or claim 42, wherein the plurality of modes includes two or more of the scan mode, the view mode, the travel mode, and the translation mode.

44. The surgical robotic system of claim 41 or claim 42, wherein the plurality of modes includes three or more of the scan mode, the view mode, the travel mode, and the translation mode.

45. The surgical robotic system of any one of claims 41 to 44, wherein the plurality of modes includes the scan mode.

46. The surgical robotic system of any one of claims 41 to 45, wherein the plurality of modes includes the view mode.

47. The surgical robotic system of any one of claims 41 to 46, wherein the plurality of modes includes the travel mode.

48. The surgical robotic system of any one of claims 41 to 47, wherein the plurality of modes includes the translation mode.

49. The surgical robotic system of any one of claims 40 to 48, wherein the surgical robotic system includes at least one pedal and wherein the control mode selection input received via the at least one pedal.

50. The surgical robotic system of any one of claims 40 to 48, wherein the surgical robotic system includes at least one pedal and wherein the control mode selection input is received via the at least one pedal or via the right hand controller or the left hand controller.

51. The surgical robotic system of any one of claims 40 to 48, wherein the first control mode selection input is received via a button or a touch input device of the right hand controller or the left hand controller or via the at least one pedal.

52. The surgical robotic system of any one of claims 41 to 51, wherein the virtual chest is defined by a chest plane extending between a first pivot point of a most proximal joint of the left robotic arm, a second pivot point of a most proximal joint of the right robotic arm, and a camera imaging center point of the camera assembly when the camera assembly is in a default position with respect to plunge.

53. The surgical robotic system of any one of claims 41 to 52, wherein the plurality of functions includes one or more of:
displaying a menu on a portion of the image display while holding positions and orientations of the camera assembly and the robotic arm assembly constant;
displaying a submenu on a portion of the image display;
selecting an option in the menu or submenu displayed on the portion of the image display;
resetting a camera assembly orientation and position to align a camera view to an average instrument tip position and to the virtual chest;
resetting a pose of the robotic assembly with respect to robotic arms and a virtual chest to a default pose while maintaining a position and an orientation of an instrument tip for each of the robotic arms;
activating a clutch with respect to a hand controller to enable movement of the respective hand controller without causing movement of the robotic assembly;
activating or exiting an instrument control mode of the surgical robotic system, a movement of one of the hand controllers when in the instrument mode causing a corresponding movement in a corresponding robotic arm of the robotic assembly;
activating or exiting an elbow bias feature, the elbow bias feature enabling adjustment of a magnitude and a direction of an elevation of an elbow of a robotic arm while maintaining a position of an instrument tip of the robotic arm for each robotic arm of the robotic assembly;
controlling an adjustment of an elevation of an elbow of a robotic arm of the robotic assembly for one robotic arm of the robotic assembly or for each robotic arm of the robotic assembly; and
changing or selecting a zoom of a view from a camera assembly of the robotic assembly.
